(19) United States
(12) Patent Application Publication
(10) Pub. No.: US 2005/0019424 A1
(43) Pub. Date: Jan. 27, 2005

(54) Anti-angiogenesis combination therapies comprising pyridazine or pyridine derivatives

(76) Inventors: Paul E. Adams, Cheshire, CT (US); Stephen J. Boyer, Fairfield, CT (US); Jacques Dumas, Bethany, CT (US); James J. Elting, Madison, CT (US); Harold C.E. Kluender, Trumbull, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/498,935

(22) PCT Filed: Dec. 20, 2002
(86) PCT No.: PCT/US02/41145

Related U.S. Application Data
(60) Provisional application No. 60/344,294, filed on Dec. 21, 2001.

(51) Int. Cl. A61K 33/24; A61K 31/501

(52) U.S. Cl. 424/649; 514/248; 514/300; 514/252.04

(57) ABSTRACT

The invention relates generally to the use of certain substituted fused or unfused pyridazine or pyridine derivatives which are KDR inhibitors in combination with other chemotherapeutic agents for use in treatment of diseases associated with abnormal angiogenesis and/or hyperpermeability and/or hyperproliferative diseases, such as cancer.

### FIELD OF THE INVENTION

The present invention relates generally to the use of certain substituted fused or unfused pyridazine or pyridine derivatives that are KDR inhibitors in combination with other chemotherapeutic agents for use in treatment of diseases associated with abnormal angiogenesis and/or hyperpermeability and/or hyperproliferative diseases, such as cancer.

### BACKGROUND OF THE INVENTION

Vasculogenesis involves the de novo formation of blood vessels from endothelial cell precursors or angioblasts. The first vascular structures in the embryo are formed by vasculogenesis. Angiogenesis involves the development of capillaries from existing blood vessels, and is the principle mechanism by which organs, such as the brain and the kidney are vascularized. While vasculogenesis is restricted to embryonic development, angiogenesis can occur in the adult, for example during pregnancy, the female cycle, or wound healing.

One major regulator of angiogenesis and vasculogenesis in both embryonic development and some angiogenic-dependent diseases is vascular endothelial growth factor (VEGF; also called vascular permeability factor, VPF). VEGF represents a family of mitogens isoforms resulting from alternative mRNA splicing and which exist in homodimeric forms. The VEGF KDR receptor is highly specific for vascular endothelial cells (for reviews, see: Farrara et al. Endocr. Rev. 1992, 13, 18; Neufield et al. FASEB J. 1999, 13, 9).

VEGF expression is induced by hypoxia (Shweiki et al. Nature 1992, 359, 843), as well as by a variety of cytokines and growth factors, such as interleukin-1, interleukin-6, epidermal growth factor and transforming growth factor-α and -β.

To date VEGF and the VEGF family members have been reported to bind to one or more of three transmembrane receptor tyrosine kinases (Mustonen et al. J. Cell Biol., 1995, 129, 895), VEGF receptor-1 (also known as flt-1 (fms-like tyrosine kinase-1)); VEGFR-2 (also known as kinase insert domain containing receptor (KDR), the murine analogue of KDR being known as fetal liver kinase-1 (flk-1)); and VEGFR-3 (also known as flt-4). KDR and flt-1 have been shown to have different signal transduction properties (Waltenberger et al. J. Biol. Chem. 1994, 269, 26988); Park et al. Oncogene 1995, 10, 135). Thus, KDR undergoes strong ligand-dependent tyrosine phosphorylation in intact cells, whereas flt-1 displays a weaker response. Thus, binding to KDR is a critical requirement for induction of the full spectrum of VEGF-mediated biological responses.

In vivo, VEGF plays a central role in vasculogenesis, and induces angiogenesis and permeabilization of blood vessels. Deregulated VEGF expression contributes to the development of a number of diseases that are characterized by abnormal angiogenesis and/or hyperpermeability processes. Regulation of the VEGF-mediated signal transduction cascade will therefore provide a useful mode for control of abnormal angiogenesis and/or hyperpermeability processes.

Angiogenesis is regarded as an absolute prerequisite for growth of tumors beyond about 1-2 mm. Oxygen and nutrients may be supplied to cells in tumors smaller than this limit through diffusion. However, every tumor is dependent on angiogenesis for continued growth after it has reached a certain size. That is, for tumors to grow beyond 3 to 4 mm³ in volume, new blood vessel growth must occur. In fact, immunchistochemical analysis of tumor sections from the margins of growing tumors show a preponderance of blood vessels, irrespective of tumor type. Tumorigenic cells within hypoxic regions of tumors respond by stimulation of VEGF production, which triggers activation of quiescent endothelial cells to stimulate new blood vessel formation (Shweiki et al. Proc. Nat'l. Acad. Sci., 1995, 92, 768). In addition, VEGF production in tumor regions where there is no angiogenesis may proceed through the ras signal transduction pathway (Grugel et al. J. Biol. Chem., 1995, 270, 25915; Rak et al. Cancer Res. 1995, 55, 4575). In situ hybridization studies have demonstrated VEGF mRNA is strongly upregulated in a wide variety of human tumors, including lung (Mattern et al. Br. J. Cancer 1996, 73, 931), thyroid (Viglietto et al. Oncogene 1995, 11, 1569), breast (Brown et al. Human Pathol. 1995, 26, 86), gastrointestional tract (Brown et al. Cancer Res. 1993, 53, 4727; Suzuki et al. Cancer Res. 1996, 56, 3004), kidney and bladder (Brown et al. Am. J. Pathol. 1993, 1431, 1255), ovary (Olson et al. Cancer Res. 1994, 54, 1255), and cervical (Guidi et al. J. Nat'l Cancer Inst. 1995, 87, 12137) carcinomas, as well as angiosacroma (Hashimoto et al. Lab. Invest. 1995, 73, 859) and several intracranial tumors (Plate et al. Nature 1992, 359, 845; Phillips et al. Int. J. Oncol. 1993, 2, 913; Berkman et al. J. Clin. Invest., 1993, 91, 153). Neutralizing monoclonal antibodies to KDR have been shown to be efficacious in blocking tumor angiogenesis (Kim et al. Nature 1993, 362, 841; Rockwell et al. Mol. Cell. Differ. 1995, 3, 315).

Cancer continues to be one of the leading causes of death in human beings. The majority of cancers are solid tumor cancers such as, without limitation, ovarian cancer, colorectal cancer, breast cancer, brain cancer, liver cancer, kidney cancer, stomach cancer, prostate cancer, lung cancer, thyroid cancer, Kaposis sarcoma and skin cancer. The primary modes of treatment of solid tumor cancers are surgery, radiation therapy and chemotherapy, separately and in combination.

Overexpression of VEGF, for example under conditions of extreme hypoxia, can also lead to intraocular angiogenesis, resulting in hyperproliferation of blood vessels, leading eventually to blindness. Such a cascade of events has been observed for a number of retinopathies, including diabetic retinopathy, ischemic retinal-vein occlusion, retinopathy of prematurity (Aiello et al. New Engl. J. Med. 1994, 331, 1480; Peer et al. Lab. Invest. 1995, 72, 638), and age-related macular degeneration (AMD; see, Lopez et al. Invest. Opththalmol. Vis. Sci. 1996, 37, 855).

In rheumatoid arthritis (RA), the in-growth of vascular pannus may be mediated by production of angiogenic factors. Levels of immunoreactive VEGF are high in the synovial fluid of RA patients, while VEGF levels were low in the synovial fluid of patients with other forms of arthritis of with degenerative joint disease (Koch et al. J. Immunol. 1994, 152, 4149). The angiogenesis inhibitor AGM-170 has been shown to prevent neovascularization of the joint in the rat collagen arthritis model (Peacock et al. J. Exper. Med. 1992, 175, 1135).

Increased VEGF expression has also been shown in psoriatic skin, as well as bullous disorders associated with subepidermal blister formation, such as bullous pemphigoid, erythema multiforme, and dermatitis herpetiformis (Brown et al. J. Invest. Dermatol. 1995, 104, 744).

Accordingly, it would be highly desirable to have efficient and essentially non toxic therapies for treating disorders associated with abnormal angiogenesis and/or hyperpermeability processes, such as proliferative diseases, e.g., cancer.

### SUMMARY OF THE INVENTION

In one embodiment, the invention is drawn to a method for treating a subject having cancer, comprising administering to the subject a therapeutically efficient amount of a first chemotherapeutic agent and a therapeutically efficient amount of a compound which is different from the first chemotherapeutic compound and having generalized structural formula I: 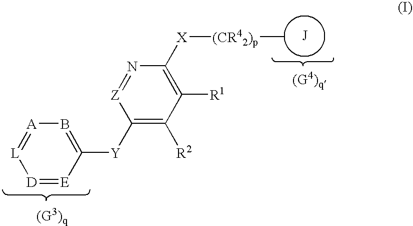
wherein R¹ and R² represent
i) independently for each occurrence H or lower alkyl;
ii) together form a bridge of structure 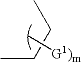
wherein binding is achieved via the terminal carbon atoms;
iii) together form a bridge of structure 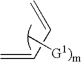
wherein binding is achieved via the terminal carbon atoms;
iv) together form a bridge of structure 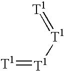
wherein one or two ring members T¹ are N and the others are CH or CG¹, and binding is achieved via the terminal atoms; or
v) together form a bridge containing two T² moieties and one T³ moiety, said bridge, taken together with the ring to which it is attached, forming a bicyclic of structure 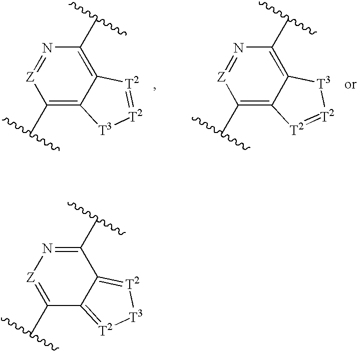
wherein
   each T² independently represents N, CH, or CG¹; and T³ represents S, O, CR⁴G¹, C(R⁴)₂, or NR³.
      G¹ is —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; —OCO₂R³; optionally substituted heteroarylalkyl; optionally substituted heteroaryloxy, —S(O)ₚ(optionally substituted heteroaryl); optionally substituted heteroarylalkyloxy; —S(O)ₚ(optionally substituted heteroarylalkyl); —CHO; —OCON(R⁶)₂; —NR³CO₂R⁶; or —NR³CON(R⁶)₂. m is 0, 1, 2, 3, or 4. R³ is H or lower alkyl. R⁴ is H, halogen, or lower alkyl. R⁶ is H; alkyl; cycloalkyl; optionally substituted aryl; optionally substituted aryl; lower alkyl; lower alkyl-N(R³)₂; or lower alkyl-OH. p is 0, 1, or 2. X is O, S, or NR³. Y is lower alkylene; —CH₂—O—; —CH₂—S—; —CH₂—NH—; —O—; —S—; —NH—; —(CR⁴₂)ₙ—S(O)ₚ-(5-membered heteroaryl)-(CR⁴₂)ₛ—; —(CR⁴₂)ₙ—C(G²)(R⁴)—(CR⁴₂)ₛ—; —O—CH₂—; —S(O)—; —S(O)₂—; —SCH₂—; —S(O)CH₂—; —S(O)₂CH₂—; —CH₂S(O)—; or —CH₂S(O)₂— wherein n and s are each independently 0 or an integer of 1-2; G² is selected from the group consisting of —CN, —CO₂R³, —CON(R⁶)₂, and —CH₂N(R⁶)₂. Z is CR⁴ or N. q is 0, 1, or 2. G³ is a monovalent or bivalent moiety and is lower alkyl; —NR³COR⁶; carboxy-substituted alkyl; lower alkoxycarbonyl-substituted alkyl; —OR⁶; —SR⁶; —S(O)R⁶; —S(O)₂R⁶; —OCOR⁶; —COR⁶; —CO₂R⁶; —CH₂OR³; —CON(R⁶)₂; —S(O)₂N(R⁶)₂; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy; —S(O)ₚ(optionally substituted heteroaryl); optionally substituted heteroarylalkyloxy; —S(O)ₚ(optionally substituted heteroarylalkyl); —OCON(R⁶)₂; —NR³CO₂R⁶; —NR³CON(R⁶)₂; or a bivalent bridge of structure T²=T²-T³ wherein each T² independently represents N, CH, or CG^{3′}; and T³ represents S, O, CR⁴G^{3′}, C(R⁴)₂, or NR³; wherein G^{3′} represents any of the above-defined moieties G³ which are monovalent; and the terminal T² is bound to L, and T³ is bound to D, forming a 5-membered fused ring. When q is 0 or each G³ is an independent lower alkyl substituent, then R¹ and R² together form a bridge containing two T² moieties and one T³ moiety, said bridge, taken together with the ring to which it is attached, forming a bicyclic of structure 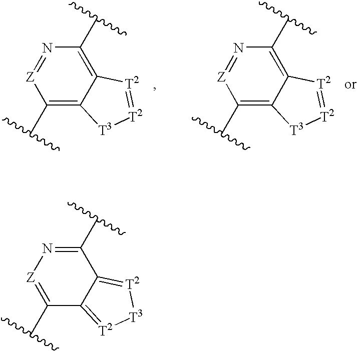
      wherein each T² independently represents N, CH, or CG¹; and T³ represents S, O, CR⁴G¹, C(R⁴)₂, or NR³. A and D independently represent N or CH. B and E independently represent N or CH. L represents N or CH; and with the provisos that a) the total number of N atoms in the ring containing A, B, D, E, and L is 0, 1, 2, or 3; and b) when L represents CH and q is 0 or any G³ is a monovalent substituent, at least one of A and D is an N atom; and c) when L represents CH and a G³ is a bivalent bridge of structure T²=T²-T³, then A, B, D, and E are also CH. J is a ring and is aryl; pyridyl; or cycloalkyl. q′ represents the number of substituents G⁴ on ring J and is 0, 1, 2, 3, 4, or 5. G⁴ is a monovalent or bivalent moiety and is —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted partially unsaturated heterocyclyl; —OCO₂R³; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —CHO; —OCON(R⁶)₂; —NR³CO₂R⁶; —NR³CON(R⁶)₂; or fused ring-forming bivalent bridges attached to and connecting adjacent positions of ring J, and having the structures: 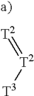
wherein
each T² independently represents N, CH, or CG^{4′};
T³ represents S, O, CR⁴G^{4′}, C(R⁴)₂, or NR³; wherein
   G4′ represents any of the above-defined moieties G⁴ which are monovalent; and binding to ring J is achieved via terminal atoms T² and T³; 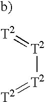
wherein
each T² independently represents N, CH, or CG^{4′}; wherein
   G4′ represents any of the above-defined moieties G⁴ which are monovalent; and with the proviso that a maximum of two bridge atoms T² may be N; and binding to ring J is achieved via terminal atoms T²; and 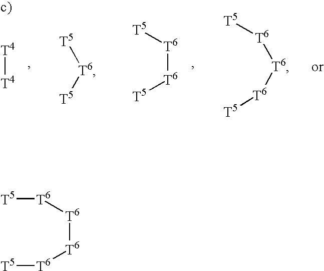
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and
binding to ring J is achieved via terminal atoms T⁴ or T⁵;
   with the provisos that:
      i) when one T⁴ is O, S, or NR³, the other T⁴ is CR⁴G^{4′} or C(R⁴)₂;
      ii) a bridge comprising T⁵ and T⁶ atoms may contain a maximum of two heteroatoms O, S, or N; and
      iii) in a bridge comprising T⁵ and T⁶ atoms, when one T⁵ group and one T⁶ group are O atoms, or two T⁶ groups are O atoms, said O atoms are separated by at least one carbon atom.
         When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members; and with the further provisos that: in G¹, G², G³, and G⁴, when two groups R³ or R⁶ are each alkyl and located on the same N atom they may be linked by a bond, an O, an S, or NR³ to form a N-containing heterocycle of 5-7 ring atoms; when an aryl, heteroaryl, or heterocyclyl ring is optionally substituted, that ring may bear up to 5 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂R³, —CHO, —CH₂OR³, —OCO₂R³, —CON(R⁶)₂, —OCON(R⁶)₂, —NR³CON(R⁶)₂, nitro, amidino, guanidino, mercapto, sulfo, and cyano; and when any alkyl group is attached to O, S, or N, and bears a hydroxyl substituent, then said hydroxyl substituent is separated by at least two carbon atoms from the O, S, or N to which the alkyl group is attached.

In a further embodiment, the invention is drawn to a method for treating a subject having cancer according to the first embodiment wherein R¹ and R² together form a bridge containing two T² moieties and one T³ moiety, said bridge, taken together with the ring to which it is attached, forming a bicyclic of structure 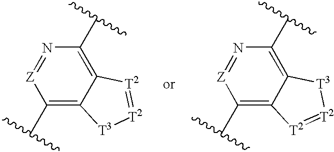
wherein each T² independently represents N, CH, or CG¹; and T³ represents S, O, CH₂, or NR³; with the proviso that when T³ is O or S, at least one T² is CH or CG¹.

In a further embodiment, the invention is drawn to a method for treating a subject having cancer according to the first embodiment wherein R¹ and R²
i) together form a bridge of structure 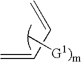
wherein binding is achieved via the terminal carbon atoms; or
ii) together form a bridge of structure 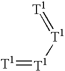
   wherein one or two ring members T¹ are N and the others are CH or CG¹, and binding is achieved via the terminal atoms.

In a further embodiment, the invention is drawn to a method for treating a subject having cancer according to the first embodiment wherein q is 1 or 2; A, B, D, and E are CH; L is N; one G³ is found on ring position D; and that G³ is —CON(R⁶)₂.

In a further embodiment, the invention is drawn to a method for treating a subject having cancer according to the first embodiment wherein q is 1 or 2; A, B, D, E, and L are CH; and one of the G³ forms a bivalent bridge of structure T²=T²-T³ wherein each T² independently represents N, CH, or CG^{3′}; and T³ represents S, O, CR⁴G^{3′}, C(R⁴)₂, or NR³; wherein G^{3′} represents any of the above-defined moieties G³ which are monovalent; and the terminal T² is bound to L, and T³ is bound to D, forming a 5-membered fused ring.

In a further embodiment, the invention is drawn to a method for treating a subject having cancer according to the embodiment two paragraphs above wherein p is 0; J is phenyl; Z is CH or N; Y is selected from a group consisting of lower alkylene; —CH₂—O—; —CH₂—S—; —CH₂—NH—; —O—; —S—; —NH—. G¹ is selected from a group consisting of —N(R⁶)₂; alkyl; amino-substituted alkyl; N-lower alkylamino-substituted alkyl; N,N-di-lower alkylamino-substituted alkyl; hydroxy-substituted alkyl; carboxy-substituted alkyl; amino-substituted alkylamino; N-lower alkylamino-substituted alkylamino; N,N-di-lower alkylamino-substituted alkylamino; hydroxy-substituted alkylamino; carboxy-substituted alkylamino; —OR⁶; —S(O)R⁶; —S(O)₂R⁶; —OCOR⁶; —COR⁶; —CO₂R⁶; —CON(R⁶)₂; —CH₂OR³; —NO₂; —CN; amidino; guanidino; sulfo; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; optionally substituted heteroarylalkyl; optionally substituted heteroaryloxy; —S(O)ₚ(optionally substituted heteroaryl); optionally substituted heteroarylalkyloxy; —S(O)ₚ(optionally substituted heteroarylalkyl); —OCON(R⁶)₂; —NR³CO₂R⁶; and —NR³CON(R⁶)₂. Any additional G³ is selected from a group consisting of halogen; lower alkyl; hydroxyl; and lower alkoxy. G⁴ is selected from a group consisting of halogen; alkyl; cycloalkyl; lower alkenyl; lower cycloalkenyl; halogen-substituted alkyl; hydroxy-substituted alkyl; cyano-substituted alkyl; lower alkoxycarbonyl-substituted alkyl; phenyl lower alkoxycarbonyl-substituted alkyl; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; —NR³CON(R⁶)₂; and fused ring-forming bivalent bridges attached to and connecting adjacent positions of ring J, said bridges having the structures: 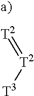
wherein each T² independently represents N, CH, or CG^{4′}; T³ represents S, O, CR⁴G^{4′}, C(R⁴)₂, or NR³; wherein G4′ represents any of the above-defined moieties G⁴ which are monovalent; and binding to ring J is achieved via terminal atoms T² and T³; 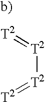
wherein each T² independently represents N, CH, or CG^{4′}; wherein G4′ represents any of the above-defined moieties G⁴ which are monovalent; and with the proviso that a maximum of two bridge atoms T² may be N; and binding to ring J is achieved via terminal atoms T²; and 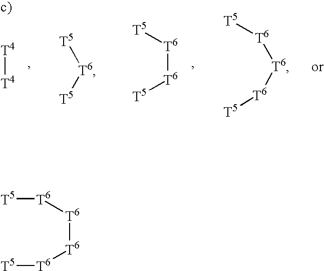
wherein each T⁴, T⁵, and T⁶ independently represents O, S, CR⁴G^{4′}, C(R⁴)₂, or NR³; wherein G4′ represents any of the above-defined moieties G⁴ which are monovalent; and binding to ring J is achieved via terminal atoms T⁴ or T⁵;
with the provisos that:
   i) when one T⁴ is O, S, or NR³, the other T⁴ is CR⁴G^{4′} or C(R⁴)₂;
   ii) a bridge comprising T⁵ and T⁶ atoms may contain a maximum of two heteroatoms O, S, or N; and
   iii) in a bridge comprising T⁵ and T⁶ atoms, when one T⁵ group and one T⁶ group are O atoms, or two T⁶ groups are O atoms, said O atoms are separated by at least one carbon atom.
      When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members.
      R¹ and R²
i) together form a bridge of structure 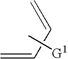
wherein binding is achieved via the terminal carbon atoms;
ii) together form a bridge of structure 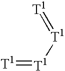
wherein one ring member T¹ is N and the others are CH or CG¹, and binding is achieved via the terminal atoms; or
iii) together form a bridge containing two T² moieties and one T³ moiety, said bridge, taken together with the ring to which it is attached, forming a bicyclic of structure 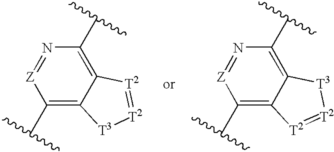
wherein
   each T² independently represents N, CH, or CG¹; and
T³ represents S, O, or NR³.

In a further embodiment, the invention is drawn to a method for treating a subject having cancer according to the embodiment two paragraphs above wherein p is 0. J is phenyl. Z is CH or N. Y is selected from a group consisting of lower alkylene; —CH₂—O—; —CH₂—S—; —CH₂—NH—; —O—; —S—; and —NH—. G¹ is selected from a group consisting of —N(R⁶)₂; alkyl; amino-substituted alkyl; N-lower alkylamino-substituted alkyl; N,N-di-lower alkylamino-substituted alkyl; hydroxy-substituted alkyl; carboxy-substituted alkyl; amino-substituted alkylamino; N-lower alkylamino-substituted alkylamino; N,N-di-lower alkylamino-substituted alkylamino; hydroxy-substituted alkylamino; carboxy-substituted alkylamino; —OR⁶; —S(O)R⁶; —S(O)₂R⁶; —OCOR⁶; —COR⁶; —CO₂R⁶; —CON(R⁶)₂; —CH₂OR³; —NO₂; —CN; amidino; guanidino; sulfo; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; and —NR³CON(R⁶)₂. Any additional G³ is selected from a group consisting of halogen; lower alkyl; hydroxyl; and lower alkoxy. G⁴ is selected from a group consisting of halogen; alkyl; cycloalkyl; lower alkenyl; lower cycloalkenyl; halogen-substituted alkyl; hydroxy-substituted alkyl; cyano-substituted alkyl; lower alkoxycarbonyl-substituted alkyl; phenyl lower alkoxycarbonyl-substituted alkyl; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —CH₂OR³; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy; —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; —NR³CON(R⁶)₂; and fused ring-forming bivalent bridges attached to and connecting adjacent positions of ring J, said bridges having the structures: 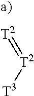
wherein each T² independently represents N, CH, or CG^{4′}; T³ represents S, O, CR⁴G^{4′}, C(R⁴)₂, or NR³; wherein G4′ represents any of the above-defined moieties G⁴ which are monovalent; and binding to ring J is achieved via terminal atoms T² and T³; 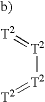
wherein each T² independently represents N, CH, or CG^{4′}; wherein G4′ represents any of the above-defined moieties G⁴ which are monovalent; and with the proviso that a maximum of two bridge atoms T² may be N; and binding to ring J is achieved via terminal atoms T²; and 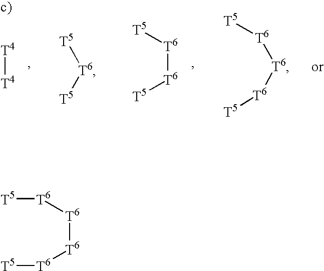
wherein each T⁴, T⁵, and T⁶ independently represents O, S, CR⁴G^{4′}, C(R⁴)₂, or NR³; wherein G4′ represents any of the above-defined moieties G⁴ which are monovalent; and binding to ring J is achieved via terminal atoms T⁴ or T⁵; with the provisos that:
   i) when one T⁴ is O, S, or NR³, the other T⁴ is CR⁴G^{4′} or C(R⁴)₂;
   ii) a bridge comprising T⁵ and T⁶ atoms may contain a maximum of two heteroatoms O, S, or N; and
   iii) in a bridge comprising T⁵ and T⁶ atoms, when one T⁵ group and one T⁶ group are O atoms, or two T⁶ groups are O atoms, said O atoms are separated by at least one carbon atom;
      When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members.
      R¹ and R²
i) together form a bridge of structure 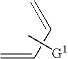
wherein binding is achieved via the terminal carbon atoms;
ii) together form a bridge of structure 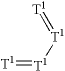
wherein one ring member T¹ is N and the others are CH or CG¹, and binding is achieved via the terminal atoms; or
iii) together form a bridge containing two T² moieties and one T³ moiety, said bridge, taken together with the ring to which it is attached, forming a bicyclic of structure 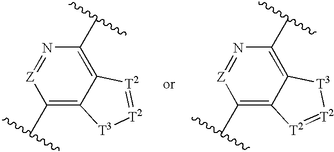
wherein each T² independently represents N, CH, or CG¹; and T³ represents S, O, or NR³.

In another embodiment, the invention is drawn to a method for treating a subject having cancer, comprising administering to the subject a therapeutically efficient amount of a first chemotherapeutic agent and therapeutically efficient amount of a compound, which is different from the first chemotherapeutic compound, having generalized structural formula III, IV, or V: 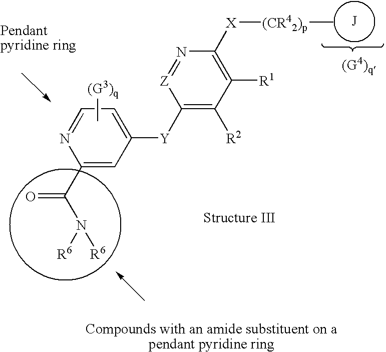
Compounds with an amide substituent on a pendant pyridine ring 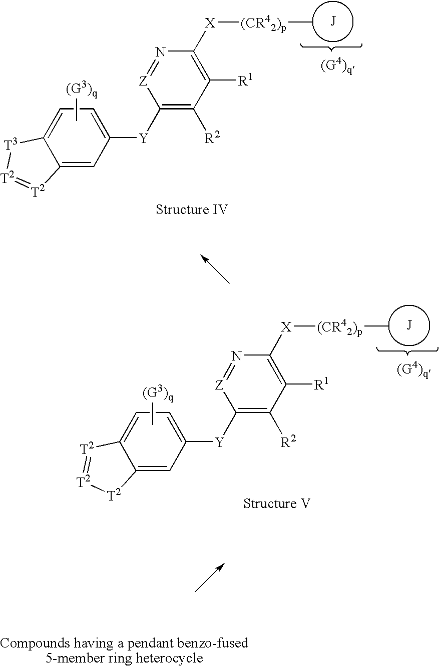
Compounds having a pendant benzo-fused 5-member ring heterocycle
   wherein R¹ and R²
i) independently represent H or lower alkyl;
ii) together form a bridge of structure 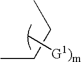
wherein binding is achieved via the terminal carbon atoms;
iii) together form a bridge of structure 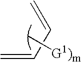
wherein binding is achieved via the terminal carbon atoms;
iv) together form a bridge of structure 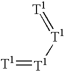
wherein one or two ring members T¹ are N and the others are CH or CG¹, and binding is achieved via the terminal atoms; or
v) together form a bridge containing T² moieties and one T³ moiety, said bridge, taken together with the ring to which it is attached, forming a bicyclic of structure 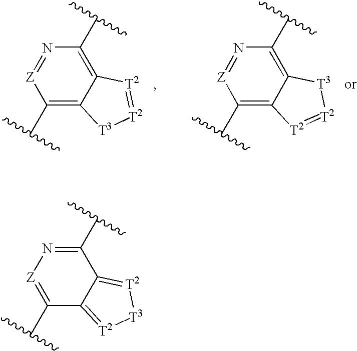
   wherein each T² independently represents N, CH, or CG¹; and T³ represents S, O, CR⁴G¹, C(R⁴)₂, or NR³. G¹ is a substituent independently selected from the group consisting of —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; —OCO₂R³; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —CHO; —OCON(R⁶)₂; —NR³CO₂R⁶; —NR³CON(R⁶)₂. R³ is H or lower alkyl. R⁶ is independently selected from the group consisting of H; alkyl; cycloalkyl; optionally substituted aryl; optionally substituted aryl lower alkyl; lower alkyl-N(R³)₂; and lower alkyl-OH. R⁴ is H, halogen, or lower alkyl. p is 0, 1, or 2. X is selected from the group consisting of O, S, and NR³. Y is selected from the group consisting of lower alkylene; —CH₂—O—; —CH₂—S—; —CH₂—NH—; —O—; —S—; —NH—; —(CR⁴₂)ₙ—S(O)ₚ-(5-membered heteroaryl)-(CR⁴₂)ₛ—; —(CR⁴₂)ₙ—C(G²)(R⁴)—(CR⁴₂)ₛ; wherein n and s are each independently 0 or an integer of 1-2; and G² is selected from the group consisting of —CN, —CO₂R³, —CON(R⁶)₂, and —CH₂N(R⁶)₂; —O—CH₂—; —S(O)—; —S(O)₂—; —SCH₂—; —S(O)CH₂—; —S(O)₂CH₂—; —CH₂S(O)—; and —CH₂S(O)₂—. Z is CH, —CG³, or N. q is 0 or 1. G³ is a monovalent moiety selected from the group consisting of lower alkyl; —NR³COR⁶; carboxy-substituted alkyl; lower alkoxycarbonyl-substituted alkyl; —OR⁶; —SR⁶; —S(O)R⁶; —S(O)₂R⁶; —OCOR⁶; —COR⁶; —CO₂R⁶; —CH₂OR³; —CON(R⁶)₂; —S(O)₂N(R⁶)₂; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; and —NR³CON(R⁶)₂. J is a ring selected from the group consisting of aryl; pyridyl; and cycloalkyl. q′ represents the number of substituents G⁴ on ring J and is 0, 1, 2, 3, 4, or 5. G⁴ is a monovalent or bivalent moiety selected from the group consisting of —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted partially unsaturated heterocyclyl; —OCO₂R³; optionally substituted heteroarylalkyl; optionally substituted heteroaryloxy, —S(O)ₚ(optionally substituted heteroaryl); optionally substituted heteroarylalkyloxy; —S(O)ₚ(optionally substituted heteroarylalkyl); —CHO; —OCON(R⁶)₂; —NR³CO₂R⁶; —NR³CON(R⁶)₂; and fused ring-forming bivalent bridges attached to and connecting adjacent positions of ring J, said bridges having the structures: 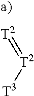
wherein
   each T² independently represents N, CH, or CG^{4′};
   T³ represents S, O, CR⁴G^{4′}, C(R⁴)₂, or NR³; wherein
      G4′ represents any of the above-defined moieties G⁴ which are monovalent; and
   binding to ring J is achieved via terminal atoms T² and T³; 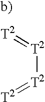
wherein each T² independently represents N, CH, or CG^{4′}; wherein G4′ represents any of the above-defined moieties G⁴ which are monovalent; and with the proviso that a maximum of two bridge atoms T² may be N; and binding to ring J is achieved via terminal atoms T²; and 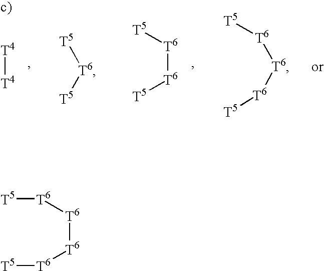
wherein each T⁴, T⁵, and T⁶ independently represents O, S, CR⁴G^{4′}, C(R⁴)₂, or NR³; wherein G4′ represents any of the above-defined moieties G⁴ which are monovalent; and binding to ring J is achieved via terminal atoms T⁴ or T⁵; with the provisos that:
   i) when one T⁴ is O, S, or NR³, the other T⁴ is CR⁴G^{4′} or C(R⁴)₂;
   ii) a bridge comprising T⁵ and T⁶ atoms may contain a maximum of two heteroatoms O, S, or N; and
iii) in a bridge comprising T⁵ and T⁶ atoms, when one T⁵ group and one T⁶ group are O atoms, or two T⁶ groups are O atoms, said O atoms are separated by at least one carbon atom.
   When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members; and with the further provisos that: in G¹, G², and G⁴, when two groups R³ or R⁶ are each alkyl and located on the same N atom they may be linked by a bond, an O, an S, or NR³ to form a N-containing heterocycle of 5-7 ring atoms. When an aryl, heteroaryl, or heterocyclyl ring is optionally substituted, that ring may bear up to 5 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂R³, —CHO, —CH₂OR³, —OCO₂R³, —CON(R⁶)₂, —OCON(R⁶)₂, —NR³CON(R⁶)₂, nitro, amidino, guanidino, mercapto, sulfo, and cyano. When any alkyl group is attached to O, S, or N, and bears a hydroxyl substituent, then said hydroxyl substituent is separated by at least two carbon atoms from the O, S, or N to which the alkyl group is attached.

In a further embodiment, the invention is drawn to a method for treating a subject having cancer according to the embodiment in the above paragraph wherein p is 0. j is phenyl or cycloalkyl. R¹ and R²
i) together form a bridge containing two T² moieties and one T³ moiety, said bridge, taken together with the ring to which it is attached, forming a bicyclic of structure 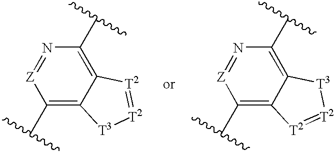
wherein each T² independently represents N, CH, or CG¹; and T³ represents S, O, CH₂, or NR³; with the proviso that when T³ is O or S, at least one T² is CH or CG¹; or
ii) together form a bridge of structure 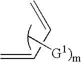
wherein binding is achieved via the terminal carbon atoms; or
iii) together form a bridge of structure 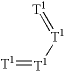
   wherein one or two ring members T¹ are N and the others are CH or CG¹, and binding is achieved via the terminal atoms.

In a further embodiment, the invention is drawn to a method for treating a subject having cancer according to the embodiment in the above paragraph wherein Y is selected from a group consisting of lower alkylene; —CH₂—O—; —CH₂—S—; —CH₂—NH—; —O—; —S—; and —NH—. G¹ is selected from a group consisting of —N(R⁶)₂; alkyl; amino-substituted alkyl; N-lower alkylamino-substituted alkyl; N,N-di-lower alkylamino-substituted alkyl; hydroxy-substituted alkyl; carboxy-substituted alkyl; amino-substituted alkylamino; N-lower alkylamino-substituted alkylamino; N,N-di-lower alkylamino-substituted alkylamino; hydroxy-substituted alkylamino; carboxy-substituted alkylamino; —OR⁶; —S(O)R⁶; —S(O)₂R⁶; —OCOR⁶; —COR⁶; —CO₂R⁶; —CON(R⁶)₂; —CH₂OR³; —NO₂; —CN; amidino; guanidino; sulfo; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; optionally substituted heteroarylalkyl; optionally substituted heteroaryloxy, —S(O)ₚ(optionally substituted heteroaryl); optionally substituted heteroarylalkyloxy; —S(O)ₚ(optionally substituted heteroarylalkyl); —OCON(R⁶)₂; —NR³CO₂R⁶; and —NR³CON(R⁶)₂.

G³ is selected from a group consisting of hydroxyl; lower alkyl; and lower alkoxy. G⁴ is selected from a group consisting of halogen; alkyl; cycloalkyl; lower alkenyl; lower cycloalkenyl; halogen-substituted alkyl; hydroxy-substituted alkyl; cyano-substituted alkyl; lower alkoxycarbonyl-substituted alkyl; phenyl lower alkoxycarbonyl-substituted alkyl; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy; —S(O)ₚ(optionally substituted heteroaryl); optionally substituted heteroarylalkyloxy; —S(O)ₚ(optionally substituted heteroarylalkyl); —OCON(R⁶)₂; —NR³CO₂R⁶; —NR³CON(R⁶)₂; and fused ring-forming bivalent bridges attached to and connecting adjacent positions of ring J, said bridges having the structures: 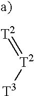
wherein each T² independently represents N, CH, or CG^{4′}; T³ represents S, O, CR⁴G^{4′}, C(R⁴)₂, or NR³; wherein G4′ represents any of the above-defined moieties G⁴ which are monovalent; and binding to ring J is achieved via terminal atoms T² and T³; 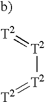
wherein each T² independently represents N, CH, or CG^{4′}; wherein G4′ represents any of the above-defined moieties G⁴ which are monovalent; and with the proviso that a maximum of two bridge atoms T² may be N; and binding to ring J is achieved via terminal atoms T²; and 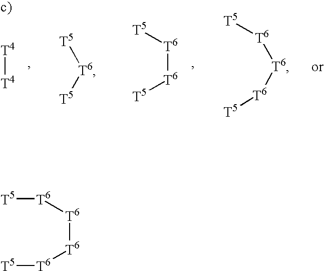
wherein each T⁴, T⁵, and T⁶ independently represents O, S, CR⁴G^{4′}, C(R⁴)₂, or NR³; wherein G4′ represents any of the above-defined moieties G⁴ which are monovalent; and binding to ring J is achieved via terminal atoms T⁴ or T⁵; with the provisos that:
   i) when one T⁴ is O, S, or NR³, the other T⁴ is CR⁴G^{4′} or C(R⁴)₂;
   ii) a bridge comprising T⁵ and T⁶ atoms may contain a maximum of two heteroatoms O, S, or N; and
   iii) in a bridge comprising T⁵ and T⁶ atoms, when one T⁵ group and one T⁶ group are O atoms, or two T⁶ groups are O atoms, said O atoms are separated by at least one carbon atom.
      When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members.

In a further embodiment, the compound is selected from the group of compounds set forth in Tables 1-4, excluding those compounds labeled as reference compounds in Table 1.

In another embodiment, the first chemotherapeutic agent and compound are administered simultaneously, wherein the compound is described according to the embodiment two paragraphs above.

In another embodiment, the first chemotherapeutic agent and compound are administered sequentially, wherein the compound is described according to the embodiment three paragraphs above.

In another embodiment, the subject is human and the compound is described according to the embodiment four paragraphs above.

In another embodiment, the subject is a non-human mammal and the compound is described according to the embodiment five paragraphs above.

### DETAILED DESCRIPTION OF THE INVENTION

Definitions

“Diseases associated with abnormal angiogenesis” refers to diseases which are initiated or aggravated by angiogenesis, such as tumors.

“P450” is used interchangeably with “cytochrome P450” and “CYP450.” The cytochromes P450 are a multi-gene family of constitutive and inducible enzymes, which have a central role in the oxidative metabolic activation and detoxification of both a wide range of xenobiotics and several groups of endogenous compounds active in cell regulation and cell signalling including arachidonic acid, steroid hormones and fatty acids (Wrighton and Stevens, Crit. Rev. Toxicol. 22, 1 (1992); Nelson et al, Pharmacogenetics 6, 1 (1996); Shimada and Guengerich, Chem. Res. Toxicol. 4, 391 (1991); Nedelcheva and Gut, Xenobiotica 24, 1151 (1994); Park et al. Pharmac. Ther. 58, 385 (1995); Capdevila et al. FASEB J. 6, 731 (1992); Miller, Endocrine Rev. 9, 295 (1988) and Oliw, Prog. Lipid Res. 33, 329 (1994)). Individual P450 forms are identified by the prefix CYP in accordance with the current P450 nomenclature (Nelson et al., supra). For example, the human CYP1 gene family, which is one of the major P450 families involved in the metabolism of xenobiotics, consists of three individual forms classified into two sub-families. The CYP1A subfamily contains two highly homologous and well characterised but distinct members: CYP1A1 and CYP1A2 (Jaiswal et al. Nucl. Acid Res. 14, 6773 (1986) and Sesardic et al. Carcinogenesis 11, 1183 (1990)).

“P450 inhibitory activity” refers to the ability of a compound to inhibit or decrease the activity of any of the P450 isoenzymes, e.g., CYP1A2, CYP2C9, CYP2C19, CYP2D6 and CYP3A4. The “activity of a P450 isoenzyme” is the ability of the enzyme to chemically modify compounds.

“Treating a subject having a disease” refers to providing treatment to the subject that will prevent, improve or cure at least one symptom of the disease.

The prefix “lower” denotes a radical having up to and including a maximum of 7 atoms, especially up to and including a maximum of 5 carbon atoms, the radicals in question being either linear or branched with single or multiple branching.

“Alkyl” means a hydrocarbon radical having up to a maximum of 12 carbon atoms, which may be linear or branched with single or multiple branching. Alkyl is especially lower alkyl.

Where the plural form is used for compounds, salts, and the like, this is taken to mean also a single compound, salt, or the like.

Any asymmetric carbon atoms may be present in the (R)-, (S)- or (R,S) configuration, preferably in the (R)- or (S)-configuration. Substituents at a double bond or a ring may be present in cis- (=Z-) or trans (=E-) form. The compounds may thus be present as mixtures of isomers or as pure isomers, preferably as enantiomer-pure diastereomers and having pure cis- or trans-double bonds.

Lower alkylene Y may be branched or linear but is preferably linear, especially methylene (—CH₂), ethylene (—CH₂—CH₂), trimethylene (—CH₂—CH₂—CH₂) or tetramethylene (—CH₂CH₂CH₂CH₂). When Y is lower alkylene, it is most preferably methylene.

“Aryl” means an aromatic radical having 6 to 14 carbon atoms, such as phenyl, naphthyl, fluorenyl or phenanthrenyl.

“Halogen” means fluorine, chlorine, bromine, or iodine but is especially fluorine, chlorine, or bromine.

“Pyridyl” means 1-, 2-, or 3-pyridyl but is especially 2- or 3-pyridyl.

“Cycloalkyl” is a saturated carbocycle that contains between 3 and 12 carbons but preferably 3 to 8 carbons.

“Cycloalkenyl” means a non-reactive and non-aromatic unsaturated carbocycle that contains between 3 and 12 carbons but preferably 3 to 8 carbons and up to three double bonds. It is well known to those skilled in the art that cycloalkenyl groups that differ from aromatics by lacking only one double bond such as cyclohaxadiene are not sufficiently non-reactive to be reasonable drug substances and therefor their use as substituents is not within the scope of this invention.

Cycloalkyl and cycloalkenyl groups may contain branch points such that they are substituted by alkyl or alkenyl groups. Examples of such branched cyclic groups are 3,4-dimethylcyclopentyl, 4-allylcyclohexyl or 3-ethylcyclopent-3-enyl.

Salts are especially the pharmaceutically acceptable salts of compounds of any of formulas I-V, such as, for example, acid addition salts, preferably with organic or inorganic acids, from compounds of any of formulas I-V with a basic nitrogen atom. Suitable inorganic acids are, for example, halogen acids such as hydrochloric acid, sulfuric acid, or phosphoric acid. Suitable organic acids are, for example, carboxylic, phosphonic, sulfonic, or sulfamic acids, for example acetic acid, propionic acid, octanoic acid, decanoic acid, dodecanoic acid, glycolic acid, lactic acid, -hydroxybutyric acid, gluconic acid, glucosemonocarboxylic acid, fumaric acid, succinic acid, adipic acid, pimelic acid, suberic acid, azeiaic acid, malic acid, tartaric acid, citric acid, glucaric acid, galactaric acid, amino acids, such as glutamic acid, aspartic acid, N-methylglycine, acetytaminoacetic acid, N-acetylasparagine or N-acetylcysteine, pyruvic acid, acetoacetic acid, phosphoserine, 2- or 3-glycerophosphoric acid.

In the definition of Y, the diradical “-(5 member heteroaryl)-” denotes a 5-membered aromatic heterocycle containing 1-3 heteroatoms selected from O, S, and N, the number of N atoms being 0-3 and the number of O and S atoms each being 0-1 and connected to the sulfur from a carbon and to —(CR⁴₂)ₛ— through a C or N atom. Examples of such diradicals include

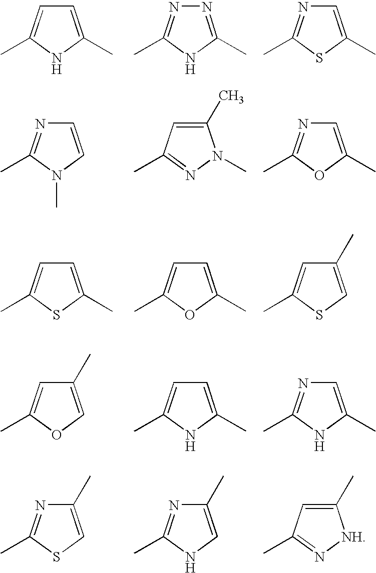

In the definitions of G¹, G², G³, and G⁴ the statement is made that when two groups R⁶ are found on a single N, they can be combined into a heterocycle of 5-7 atoms. Examples of such heterocycles, including the N to which they are attached, are:

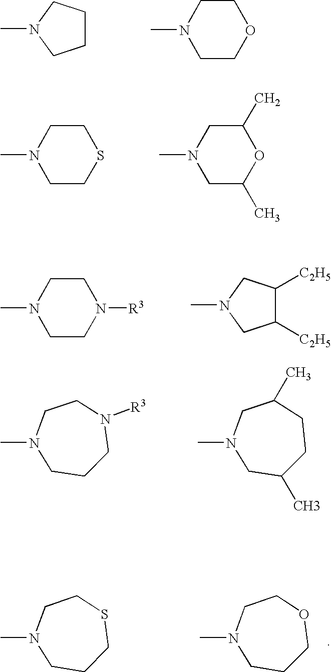

“Heterocyclyl” or “heterocycle” means a five- to seven-membered heterocyclic system with 1-3 heteroatoms selected from the group nitrogen, oxygen, and sulfur, which may be unsaturated or wholly or partly saturated, and is unsubstituted or substituted especially by lower alkyl, such as methyl, ethyl, 1-propyl, 2-propyl, or tert-butyl.

When an aryl, heteroaryl, or heterocyclyl ring is said to be optionally substituted, that ring may bear up to 5 substituents which are independently selected from the group consisting of amino, mono- or di-loweralkyl-substituted amino, lower alkanoylamino, halogeno, lower alkyl, halogenated lower alkyl such as trifluoromethyl, hydroxy, lower alkoxy, lower alkylthio, halogenated lower alkoxy such as trifluoromethoxy, halogenated lower alkylthio such as trifluoromethylthio, lower alkanoyloxy, —CO₂R³, —CHO, —CH₂OR³, —OCO₂R³, —CON(R⁶)₂, —OCON(R⁶)₂, —NR³CON(R⁶)₂, nitro, amidino, guanidino, mercapto, sulfo, and cyano.

In the ring attached to Y, the ring members A, B, D, E, and L may be N or CH, it being understood that the optional substituents G³ are necessarily attached to carbon and not nitrogen, and that when a given carbon bears a substituent group G³, that G³ group is in place of the H atom the carbon would bear in the absence of the G³ group. When a subsituent G³ is a bivalent substituent, it counts as one substituent even though it is necessarily attached to two adjacent carbons on the ring comprising A, B, D, E, L, and the carbon attached to Y.

Examples of ring J together with two adjacent G⁴ moieties which taken together form a second fused ring are:

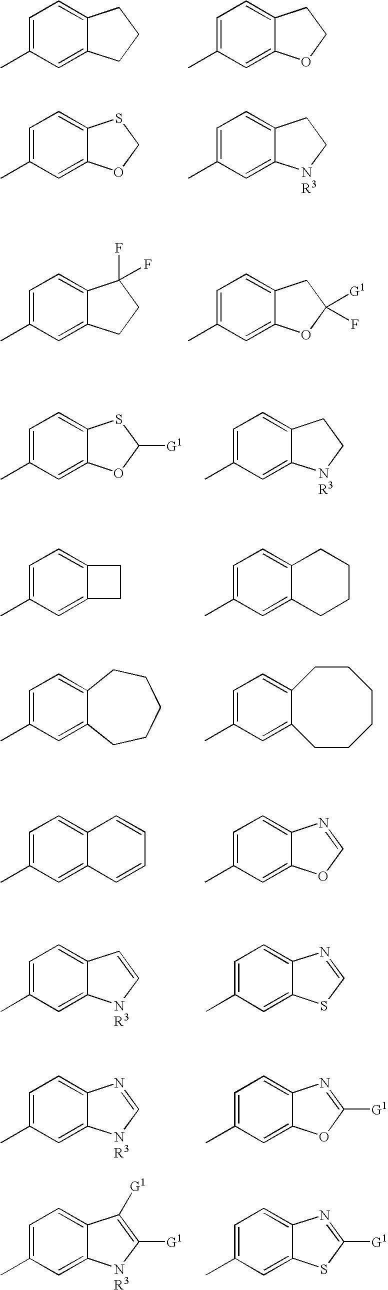

“Heteroaryl” means a monocyclic or fused bicyclic aromatic system with between 5 and 10 atoms in total of which 1-4 are heteroatoms selected from the group comprising nitrogen, oxygen, and sulfur and with the remainder being carbon. Heteroaryl is preferably a monocyclic system with 5 or 6 atoms in total, of which 1-3 are heteroatoms.

“Alkenyl” means an unsaturated radical having up to a maximum of 12 carbon atoms and may be linear or branched with single or multiple branching and containing up to 3 double bonds. Alkenyl is especially lower alkenyl with up to 2 double bonds.

“Alkanoyl” means alkylcarbonyl, and is especially lower alkylcarbonyl.

Halogenated lower alkyl, halogenated lower alkoxy and halogenated lower alkylthio are substituents in which the alkyl moieties are substituted either partially or in full with halogens, preferably with chlorine and/or fluorine and most preferably with fluorine. Examples of such substituents are trifluoromethyl, trifluoromethoxy, trifluoromethylthio, 1,1,2,2-tetrafluoroethoxy, dichloromethyl, fluoromethyl and difluoromethyl.

When a substituent is named as a string of fragments such as “phenyl-lower alkoxycarbonyl-substituted alkylamino,” it is understood that the point of attachment is to the final moiety of that string (in this case amino) and that the other fragments of that string are connected to each other in sequence as they are listed in the string. Thus an example of “phenyl-lower alkoxycarbonyl-substituted alkylamino” is:

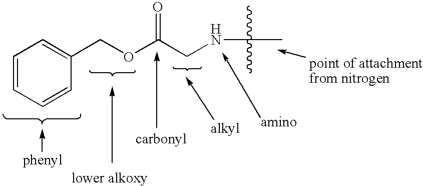

When a substituent is named as a string of fragments with a bond at the start (typically written as a dash) such as “—S(O)ₚ(optionally substituted heteroarylalkyl)”, it is understood that the point of attachment is to the first atom of that string (in this case S or sulfur) and that the other fragments of that string are connected to each other in sequence as they are listed in the string. Thus an example of “—S(O)ₚ(optionally substituted heteroarylalkyl)” is:

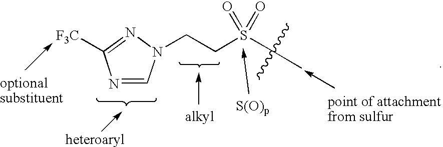

It is to be understood that the left-most moiety of each of the varients of the linker Y is connected to the ring containing A, B, D, E, and L and that the right-most moiety of the linker is connected to the pyridazine fragment of the generalized formulae. Thus examples of the use of the linker “—CH₂—O—” or of the linker “—O—CH₂—” are represented in the following invention compounds:

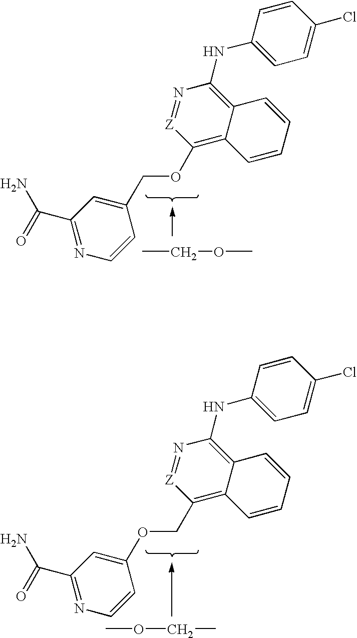

When a variable group or substituent with a given symbol (i.e., R³, R⁴, R⁶, G¹, G², G³ or G⁴) is used more than once in a given structure, it is to be understood that each of these groups or substituents may be independently varied within the range of definitions for that symbol.

Exemplary Compounds

The invention provides compounds having the generalized structural formula I: 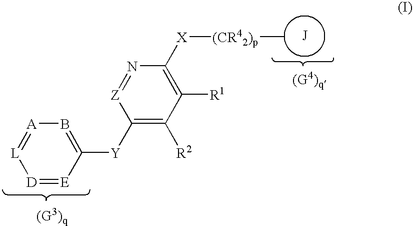
wherein
R¹ and R² represent
i) independently for each occurrence H or lower alkyl;
ii) together form a bridge of structure 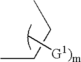
wherein binding is achieved via the terminal carbon atoms;
iii) together form a bridge of structure 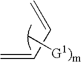
wherein binding is achieved via the terminal carbon atoms;
iv) together form a bridge of structure 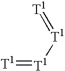
wherein one or two ring members T¹ are N and the others are CH or CG¹, and binding is achieved via the terminal atoms; or
v) together form a bridge containing two T² moieties and one T³ moiety, said bridge, taken together with the ring to which it is attached, forming a bicyclic of structure 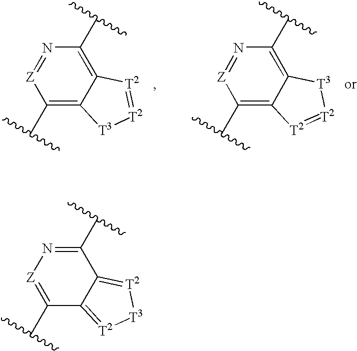
wherein
   each T² independently represents N, CH, or CG¹; and
   T³ represents S, O, CR⁴G¹, C(R⁴)₂, or NR³.

G¹ is —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; —OCO₂R³; optionally substituted heteroarylalkyl; optionally substituted heteroaryloxy; —S(O)ₚ(optionally substituted heteroaryl); optionally substituted heteroarylalkyloxy; —S(O)ₚ(optionally substituted heteroarylalkyl); —CHO; —OCON(R⁶)₂; —NR³CO₂R⁶; or —NR³CON(R⁶)₂.

m is 0, 1, 2, 3, or 4.

R³ is H or lower alkyl.

R⁴ is H, halogen, or lower alkyl.

R⁶ is H; alkyl; cycloalkyl; optionally substituted aryl; optionally substituted aryl; lower alkyl; lower alkyl-N(R³)₂; or lower alkyl-OH.

p is 0, 1, or 2.

X is O, S, or NR³;

Y is lower alkylene; —CH₂—O—; —CH₂—S—; —CH₂—NH—; —O—; —S—; —NH—; —(CR⁴₂)ₙ—S(O)ₚ-(5-membered heteroaryl)-(CR⁴₂)ₛ—; —(CR⁴₂)ₙ—C(G²)(R⁴)—(CR⁴₂)ₛ—; —O—CH₂—; —S(O)—; —S(O)₂—; —SCH₂—; —S(O)CH₂—; —S(O)₂CH₂—; —CH₂S(O)—; or —CH₂S(O)₂—
wherein
   n and s are each independently 0 or an integer of 1-2;
   G² is selected from the group consisting of —CN, —CO₂R³, —CON(R⁶)₂, and —CH₂N(R⁶)₂;

Z is CR⁴ or N.

q is 0, 1, or 2.

G³ is a monovalent or bivalent moiety and is lower alkyl; —NR³COR⁶; carboxy-substituted alkyl; lower alkoxycarbonyl-substituted alkyl; —OR⁶; —SR⁶; —S(O)R⁶; —S(O)₂R⁶; —OCOR⁶; —COR⁶; —CO₂R⁶; —CH₂OR³; —CON(R⁶)₂; —S(O)₂N(R⁶)₂; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; —NR³CON(R⁶)₂; or a bivalent bridge of structure T²=T²-T³
wherein
   each T² independently represents N, CH, or CG³; and
      T³ represents S, O, CR⁴G^{3′}, C(R⁴)₂, or NR³; wherein
      G³ represents any of the above-defined moieties G³ which are monovalent; and the terminal T² is bound to L, and T³ is bound to D, forming a 5-membered fused ring.

A and D independently represent N or CH.

B and E independently represent N or CH.

L represents N or CH; and with the provisos that
a) the total number of N atoms in the ring containing A, B, D, E, and L is 0, 1, 2, or 3; and
b) when L represents CH and q is 0 or any G³ is a monovalent substituent, at least one of A and D is an N atom; and
c) when L represents CH and a G³ is a bivalent bridge of structure T²=T²-T³, then A, B, D, and E are also CH.

J is a ring and is aryl; pyridyl; or cycloalkyl.

q′ represents the number of substituents G⁴ on ring J and is 0, 1, 2, 3, 4, or 5.

G⁴ is a monovalent or bivalent moiety and is —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; ammo-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted partially unsaturated heterocyclyl; —OCO₂R³; optionally substituted heteroarylalkyl; optionally substituted heteroaryloxy; —S(O)ₚ(optionally substituted heteroaryl); optionally substituted heteroarylalkyloxy; —S(O)ₚ(optionally substituted heteroarylalkyl); —CHO; —OCON(R⁶)₂; —NR³CO₂R⁶; —NR³CON(R⁶)₂; or fused ring-forming bivalent bridges attached to and connecting adjacent positions of ring J, and having the structures: 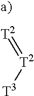
wherein
each T² independently represents N, CH, or CG^{4′};
T³ represents S, O, CR⁴G^{4′}, C(R⁴)₂, or NR³; wherein
   G4′ represents any of the above-defined moieties G⁴ which are monovalent; and binding to ring J is achieved via terminal atoms T² and T³; 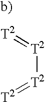
wherein
each T² independently represents N, CH, or CG^{4′}; wherein
   G4′ represents any of the above-defined moieties G⁴ which are monovalent; and with the proviso that a maximum of two bridge atoms T² may be N; and binding to ring J is achieved via terminal atoms T²; and 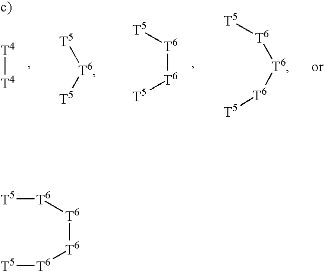
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and binding to ring J is achieved via terminal atoms T⁴ or T⁵e;
   with the provisos that:
      i) when one T⁴ is O, S, or NR³, the other T⁴ is CR⁴G^{4′} or C(R⁴)₂;
      ii) a bridge comprising T⁵ and T⁶ atoms may contain a maximum of two heteroatoms O, S, or N; and
      iii) in a bridge comprising T⁵ and T⁶ atoms, when one T⁵ group and one T⁶ group are O atoms, or two T⁶ groups are O atoms, said O atoms are separated by at least one carbon atom.

When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members; and with the further provisos that:
in G¹, G², G³, and G⁴, when two groups R³ or R⁶ are each alkyl and located on the same N atom they may be linked by a bond, an O, an S, or NR³ to form a N-containing heterocycle of 5-7 ring atoms;
when an aryl, heteroaryl, or heterocyclyl ring is optionally substituted, that ring may bear up to 5 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂³, —CHO, —CH₂OR³, —OCO₂R³, —CON(R⁶⁾₂, —OCON(R⁶)₂, —NR³CON(R⁶)₂, nitro, amidino, guanidino, mercapto, sulfo, and cyano; and
when any alkyl group is attached to O, S, or N, and bears a hydroxyl substituent, then said hydroxyl substituent is separated by at least two carbon atoms from the O, S, or N to which the alkyl group is attached.

In another embodiment of the invention the variables of formula I are defined as follows.
R¹ and R²:
i) together form a bridge of structure 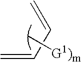
wherein binding is achieved via the terminal carbon atoms; or
ii) together form a bridge of structure 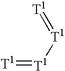
wherein one of the ring members T¹ is N and the others are CH, and binding is achieved via the terminal atoms; or
iii) together form a bridge containing two T² moieties and one T³ moiety, said bridge, taken together with the ring to which it is attached, forming a bicyclic of structure 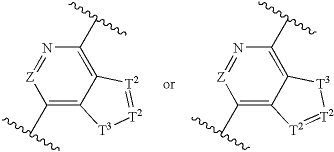
wherein
   each T² independently represents N, CH, or CG¹;
   T³ represents S, O, CH₂, or NR³; and
   with the proviso that when T³ is O or S, at least one T² is CH or CG¹.

In one embodiment, in the bridge in iii), the terminal T² is N or CH, the non-terminal T² is CH or CG¹, and T³ is S or O.

m is 0, 1, or 2. In another embodiment m is 0.

In one embodiment G¹ is located on a non-terminal atom of the bridge and is selected from the group consisting of —N(R⁶)₂; —NR³COR⁶; halogen; alkyl; lower alkyl; hydroxy-substituted alkyl; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; carboxy-substituted alkylamino; lower alkoxycarbonyl-substituted alkylamino; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —NO₂; —CN; optionally substituted heteroarylalkyl; optionally substituted heteroaryloxy; —S(O)ₚ(optionally substituted heteroaryl); optionally substituted heteroarylalkyloxy, and —S(O)ₚ(optionally substituted heteroarylalkyl). In one embodiment G¹ is a substituent independently selected from the group consisting of —N(R⁶)₂; —NR³COR⁶; halogen; —OR⁶ wherein R⁶ represents lower alkyl; —NO₂; optionally substituted heteroaryloxy; and optionally substituted heteroarylalkyloxy.

R³ is H or lower alkyl;

R⁴ is H.

R⁶ is independently selected from the group consisting of H; lower alkyl; optionally substituted aryl; and optionally substituted aryl lower alkyl.

p is 0 or 1.

X is NR³.

Y is selected from the group consisting of lower alkylene, optionally substituted by OH; —CH₂—O—; —CH₂—S—; —CH₂—NH—; —S—; —NH—; —(CR⁴₂)ₙ—S(O)ₚ-(5-membered heteroaryl)-(CR⁴₂)ₛ—; —(CR⁴₂)ₙ—C(G²)(R⁴)—(CR⁴₂)ₛ—; —O—CH₂—; —S(O)—; and —S(O)₂—. In one embodiment, Y is selected from the group consisting of —CH₂—O—; —CH₂—NH—; —S—; —NH—; —(CR⁴₂)ₙ—S(O)ₚ-(5-membered heteroaryl)-(CR⁴₂)ₛ—; and —O—CH₂—.

n and s are 0.

A, B, D, and E are CH or N, and L is N or CH, with the provisos that when L is N, any substituents G³ are monovalent, and when L is CH then any substituents G³ are divalent making this ring a pyridine; the total number of N atoms in the ring containing A, D, and L is 1 or 2; and when L is CH, at least one of A and D is an N atom.

G³ is selected from the group consisting of monovalent moieties lower alkyl; —NR³COR⁶; —OR⁶; —SR⁶; —S(O)R⁶; —S(O)₂R⁶; —CO₂R⁶; —CON(R⁶)₂; —S(O)₂N(R⁶)₂; —CN; optionally substituted aryl; optionally substituted heteroaryl; optionally substituted heteroarylalkyl; optionally substituted heteroaryloxy; —S(O)ₚ(optionally substituted heteroaryl); optionally substituted heteroarylalkyloxy, —S(O)ₚ(optionally substituted heteroarylalkyl); and bivalent bridge of structure T²=T²-T³ wherein T² represents N or CH. T³ is preferably S, O, CR⁴, or NR³. In one embodiment, G³ is selected from the group consisting of monovalent moieties lower alkyl; —NR³COR⁶; —CO₂R⁶; —CON(R⁶)₂; —S(O)₂N(R⁶)₂; and bivalent bridge of structure T²=T²-T³ wherein T² represents N or CH. Most preferably T³ is S, O, CH₂, or NR³.

q is 0, 1, or 2.

J is a phenyl ring.

q′ is 0, 1, 2, or 3. In another embodiment q′ is 1 or 2.

G⁴ is selected from the group consisting of —N(R⁶)₂; —NR³COR⁶; halogen; alkyl; halogen-substituted alkyl; hydroxy-substituted alkyl; carboxy-substituted alkyl; lower alkoxycarbonyl-substituted alkyl; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —CH₂OR³; —NO₂; —CN; optionally substituted heteroarylalkyl; optionally substituted heteroaryloxy; —S(O)ₚ(optionally substituted heteroaryl); optionally substituted heteroarylalkyloxy, —S(O)ₚ(optionally substituted heteroarylalkyl); as well as fused ring-forming bridges attached to and connecting adjacent positions of the phenyl ring, said bridges having the structures: 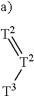
wherein each T² independently represents N, CG⁴ or CH; T³ represents S, CHG⁴, CH₂, NR³, or O; and binding to the phenyl ring is achieved via terminal atoms T² and T³; 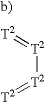
wherein each T² independently represents N, CH, or CG^{4′}; with the proviso that a maximum of two bridge atoms T² may be N; and binding to the phenyl ring is achieved via terminal atoms T²; and 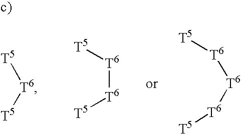
wherein each T⁵, and T⁶ independently represents O, S, CHG⁴, NR³ or CH₂; and binding to the phenyl ring J is achieved via terminal atoms T⁵; with the provisos that:
   i) a bridge comprising T⁵ and T⁶ atoms may contain a maximum of two heteroatoms O, S, or N; and
   ii) in a bridge comprising T⁵ and T⁶ atoms, when one T⁵ group and one T⁶ group are O atoms, or two T⁶ groups are O atoms, said O atoms are separated by at least one carbon atom.

In one embodiment alkyl groups which constitute all or part of a G⁴ moiety are lower alkyl.

When G⁴ is an alkyl group located on ring J adjacent to the linkage —(CR⁴)ₚ—, and X is NR³ wherein R³ is an alkyl substituent, then G⁴ and the alkyl substituent R³ on X may be joined to form a bridge of structure —(CH₂)_{p′}— wherein p′ is 2 or 3, with the proviso that the sum of p and p′ is 2 or 3, resulting in formation of a nitrogen-containing ring of 5 or 6 members. In another embodiment, the sum of p and p′ is 2, resulting in formation of a 5-membered ring.

In another embodiment, in G¹, G², G³, and G⁴, when two groups R⁶ are each alkyl and located on the same N atom they may be linked by a bond, an O, an S, or NR³ to form a N-containing heterocycle of 5-6 ring atoms.

In another embodiment, when an aryl, heteroaryl, or heterocyclyl ring is optionally substituted, that ring may bear up to 2 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂R³, —CH₂OR³, —OCO₂R³, —CON(R⁶)₂, —NR³CON(R⁶)₂ nitro, and cyano.

In another embodiment, when an aryl, heteroaryl, or heterocyclyl ring is optionally substituted, that ring may bear up to 2 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, —CO₂R³, —CON(R⁶)₂, nitro, and cyano.

In another embodiment the method comprises a compound having structural formula I and the accompanying definitions with the proviso that when q is 0 or each G³ is an independent lower alkyl substituent, then R¹ and R² together form a bridge containing two T² moieties and one T³ moiety, said bridge, taken together with the ring to which it is attached, forming a bicyclic of structure 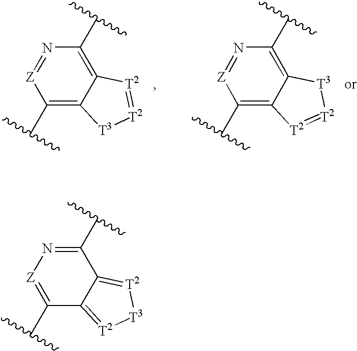
wherein each T² independently represents N, CH, or CG¹; and T³ represents S, O, CR⁴G¹, C(R⁴)₂, or NR³.

In another embodiment the method comprises a compound having structural formula I and the accompanying definitions wherein R¹ and R² together form a bridge containing two T² moieties and one T³ moiety, taken together with the ring to which it is attached, form a bicyclic of structure 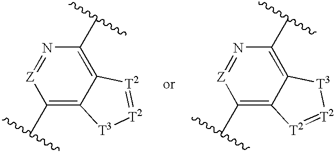
wherein each T² independently represents N, CH, or CG¹; and T³ represents S, O, CH₂, or NR³; with the proviso that when T³ is O or S, at least one T² is CH or CG¹.

In another embodiment the method comprises a compound having general formula I and the accompanying definitions wherein R¹ and R²
i) together form a bridge of structure 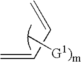
wherein binding is achieved via the terminal carbon atoms; or
ii) together form a bridge of structure 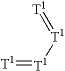
wherein one or two ring members T¹ are N and the others are CH or CG¹, and binding is achieved via the terminal atoms.

In another embodiment the method comprises a compound having general structural formula I and the accompanying definitions wherein q is 1 or 2; A, B, D, and E are CH; L is N; one G³ is found on ring position D; and that G³ is —CON(R⁶)₂.

In another embodiment the method comprises a compound having general structural formula I and the accompanying definitions wherein q is 1 or 2; A, B, D, E, and L are CH; and one of the G³ forms a bivalent bridge of structure T²=T²-T³ wherein each T² independently represents N, CH, or CG^{3′}; and T³ represents S, O, CR⁴G^{3′}, C(R⁴)₂, or NR³; wherein G^{3′} represents any of the above defined moieties G³ which are monovalent; and the terminal T² is bound to L, and T³ is bound to D, forming a 5-membered fused ring.

In another embodiment the method comprises a compound having general structural formula I and the accompanying definitions and wherein p is 0. j is phenyl. Z is CH or N. Y is selected from the group consisting of lower alkylene; —CH₂—O—; —CH₂—S—; —CH₂—NH—; —O—; —S—; and —NH—. G¹ is selected from a group consisting of —N(R⁶)₂; alkyl; amino-substituted alkyl; N-lower alkylamino-substituted alkyl; N,N-di-lower alkylamino-substituted alkyl; hydroxy-substituted alkyl; carboxy-substituted alkyl; amino-substituted alkylamino; N-lower alkylamino-substituted alkylamino; N,N-di-lower alkylamino-substituted alkylamino; hydroxy-substituted alkylamino; carboxy-substituted alkylamino; —OR⁶; —S(O)R⁶; —S(O)₂R⁶; —OCOR⁶; —COR⁶; —CO₂R⁶; —CON(R⁶)₂; —CH₂OR³; —NO₂; —CN; amidino; guanidino; sulfo; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; optionally substituted heteroarylalkyl; optionally substituted heteroaryloxy; —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; and —NR³CON(R⁶)₂. G³ is selected from a group consisting of halogen; lower alkyl; hydroxyl; and lower alkoxy. G⁴ is selected from a group consisting of halogen; alkyl; cycloalkyl; lower alkenyl; lower cycloalkenyl; halogen-substituted alkyl; hydroxy-substituted alkyl; cyano-substituted alkyl; lower alkoxycarbonyl-substituted alkyl; phenyl lower alkoxycarbonyl-substituted alkyl; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂⁶; —CON(R⁶)₂; —CH₂OR³; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy; —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; —NR³CON(R⁶)₂; and fused ring-forming bivalent bridges attached to and connecting adjacent positions of ring J, said bridges having the structures: 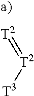
wherein
   each T² independently represents N, CH, or CG^{4′};
   T³ represents S, O, CR⁴G^{4′}, C(R⁴)₂, or NR³; wherein
      G4′ represents any of the above-defined moieties G⁴ which are monovalent; and
   binding to ring J is achieved via terminal atoms T² and T³; 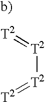
wherein
   each T² independently represents N, CH, or CG^{4′}; wherein
      G4′ represents any of the above-defined moieties G⁴ which are monovalent; and
   with the proviso that a maximum of two bridge atoms T² may be N; and
   binding to ring J is achieved via terminal atoms T²; and 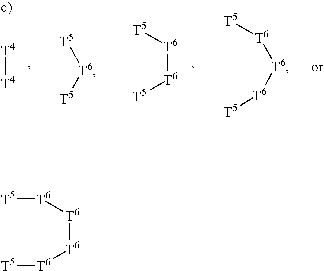
wherein
   each T⁴, T⁵, and T⁶ independently represents O, S, CR⁴G^{4′}, C(R⁴)₂, or NR³; wherein
      G4′ represents any of the above-defined moieties G⁴ which are monovalent; and
   binding to ring J is achieved via terminal atoms T⁴ or T⁵;
   with the provisos that:
      i) when one T⁴ is O, S, or NR³, the other T⁴ is CR⁴G^{4′} or C(R⁴)₂;
      ii) a bridge comprising T⁵ and T⁶ atoms may contain a maximum of two heteroatoms O, S, or N; and
      iii) in a bridge comprising T⁵ and T⁶ atoms, when one T⁵ group and one T⁶ group are O atoms, or two T⁶ groups are O atoms, said O atoms are separated by at least one carbon atom;
         when G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members. R¹ and R²
i) together form a bridge of structure 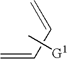
wherein binding is achieved via the terminal carbon atoms;
ii) together form a bridge of structure 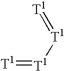
wherein one ring member T¹ is N and the others are CH or CG¹, and binding is achieved via the terminal atoms; or
iii) together form a bridge containing two T² moieties and one T³ moiety, said bridge, taken together with the ring to which it is attached, forming a bicyclic of structure 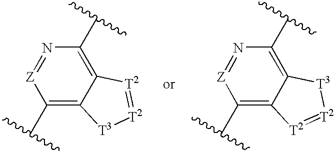
wherein each T² independently represents N, CH, or CG¹; and
   T³ represents S, O, or NR³.

In another embodiment the method comprises a compound of general structural formula I and the accompanying definitions wherein p is 0. j is phenyl. Z is CH or N. Y is selected from a group consisting of lower alkylene; —CH₂—O—; —CH₂—S—; —CH₂—N—; —O—; —S—; and —NH—. G¹ is selected from a group consisting of —N(R⁶)₂; alkyl; amino-substituted allyl; N-lower alkylamino-substituted alkyl; N,N-di-lower alkylamino-substituted alkyl; hydroxy-substituted alkyl; carboxy-substituted alkyl; amino-substituted alkylamino; N-lower alkylamino-substituted alkylamino; N,N-di-lower alkylamino-substituted alkylamino; hydroxy-substituted alkylamino; carboxy-substituted alkylamino; —OR⁶; —S(O)R⁶; —S(O)₂R⁶; —OCOR⁶; —COR⁶; —CO₂R⁶; —CON(R⁶)₂; —CH₂OR³; —NO₂; —CN; amidino; guanidino; sulfo; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; and —NR³CON(R⁶)₂. G³ is selected from a group consisting of halogen; lower alkyl; hydroxyl; and lower alkoxy. G⁴ is selected from a group consisting of halogen; alkyl; cycloalkyl; lower alkenyl; lower cycloalkenyl; halogen-substituted alkyl; hydroxy-substituted alkyl; cyano-substituted alkyl; lower alkoxycarbonyl-substituted alkyl; phenyl lower alkoxycarbonyl-substituted alkyl; —OR⁶; —SR⁶; —S(O)R⁶; —S(O)₂R⁶; halogenated lower alkoxy; halogenated lower alkylthio; halogenated lower alkylsulfonyl; —OCOR⁶; —COR⁶; —CO₂R⁶; —CON(R⁶)₂; —CH₂OR³; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy; —S(O)ₚ(optionally substituted heteroaryl); optionally substituted heteroarylalkyloxy, —S(O)ₚ(optionally substituted heteroarylalkyl); —OCON(R⁶)₂; NR³CO₂R⁶; —NR³CON(R⁶)₂; and fused ring-forming bivalent bridges attached to and connecting adjacent positions of ring J, said bridges having the structures: 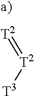
wherein
   each T² independently represents N, CH, or CG^{4′};
   T³ represents S, O, CR⁴G^{4′}, C(R⁴)₂, or NR³; wherein
      G4′ represents any of the above-defined moieties G⁴ which are monovalent; and
   binding to ring J is achieved via terminal atoms T² and T³; 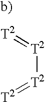
wherein
   each T² independently represents N, CH, or CG^{4′}; wherein
      G4′ represents any of the above-defined moieties G⁴ which are monovalent; and
   with the proviso that a maximum of two bridge atoms T² may be N; and
   binding to ring J is achieved via terminal atoms T²; and 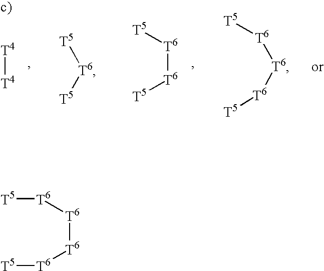
wherein
   each T⁴, T⁵, and T⁶ independently represents O, S, CR⁴G^{4′}, C(R⁴)₂, or NR³; wherein
      G4′ represents any of the above-defined moieties G⁴ which are monovalent; and
   binding to ring J is achieved via terminal atoms T⁴ or T⁵;
   with the provisos that:
      i) when one T⁴ is O, S, or NR³, the other T⁴ is CR⁴G^{4′} or C(R⁴)₂;
      ii) a bridge comprising T⁵ and T⁶ atoms may contain a maximum of two heteroatoms O, S, or N; and
      iii) in a bridge comprising T⁵ and T⁶ atoms, when one T⁵ group and one T⁶ group are O atoms, or two T⁶ groups are O atoms, said O atoms are separated by at least one carbon atom;
         when G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members. R¹ and R²
i) together form a bridge of structure 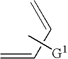
wherein binding is achieved via the terminal carbon atoms;
ii) together form a bridge of structure 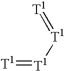
wherein one ring member T¹ is N and the others are CH or CG¹, and binding is achieved via the terminal atoms; or
iii) together form a bridge containing two T² moieties and one T³ moiety, said bridge, taken together with the ring to which it is attached, forming a bicyclic of structure 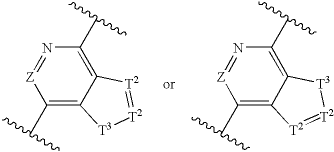
wherein each T² independently represents N, CH, or CG¹; and
   T³ represents S, O, or NR³.

In a preferred embodiment of the invention, the combination therapy includes an anti-angiogenic compound of the invention having a low P450 isoenzyme inhibitory activity. Indeed, combination therapy is expected to be particularly effective if the compounds (or agents or drugs) that are co-administered utilize different mechanisms of action to compliment each other's efficacy. In particular, given the necessity of neovascularization for the growth of solid tumors and the role of VEGF activation of KDR as one of the most important mediators of angiogenesis, compounds capable of inhibiting the angiogenic effect of KDR activation would be expected to compliment standard cytotoxic chemotherapy; that is, utilize different mechanisms of action to increase the efficacy of the overall treatment without additional toxicity that would have come from higher dosing of either agent alone to achieve the same end effect. That assumes, however, that either the two or more agents are given at different time intervals or that there exist no drug-drug interactions between the KDR inhibitor and the other chemotherapeutic agent(s) when given together. A common cause of drug-drug interactions is the inhibition by either of the active agents of various metabolizing enzymes such as those of the cytochrome P450 group. Inhibition by one of the active agents of a major metabolizing enzyme of the other agent can cause that second agent to increase in concentration to toxic levels. Accordingly, anti-angiogenic compounds of the invention which have a low P450 inhibitory activity are expected to have a low potential to result in drug drug interactions when given in combination with other chemotherapeutic agents.

Surprisingly, compounds described herein which have 2-aminocarbonyl (amide) substituents on the pendant pyridine ring show low levels of P450 inhibition wherein analogous compounds described herein or in applications WO 98/35958; WO 00/09495 and WO 01/58899, having a pendant pyridine ring but without the amide substituent, generally show much higher P450 inhibition values (low IC₅₀s) (see Examples). It has also been found that compounds described herein that have pendant benzo-fused 5 member ring heterocycles rather than pendant pyridine rings also do not exhibit high inhibition of P450 enzymes.

Accordingly, preferred compounds with low P450 inhibitory activity include pyridazine or pyridine derivatives with pendant pyridine rings as shown herein that, in addition, have aminocarbonyl (amide) substituents on the carbon adjacent to the pendant pyridine nitrogen. Other preferred compounds include those described herein that lack a pendant pyridine ring, but which have a benzo-fused-5-member ring heterocycle.

Exemplary compounds having low P450 inhibitory activity are set forth below: 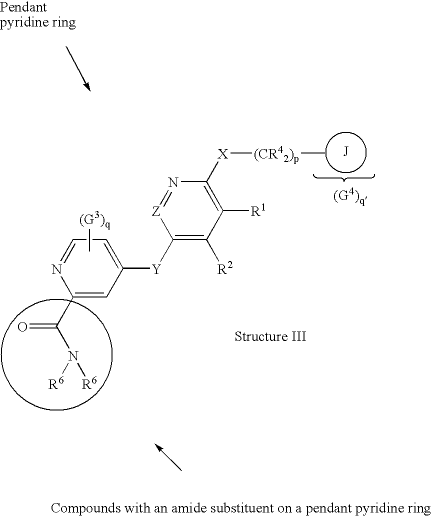 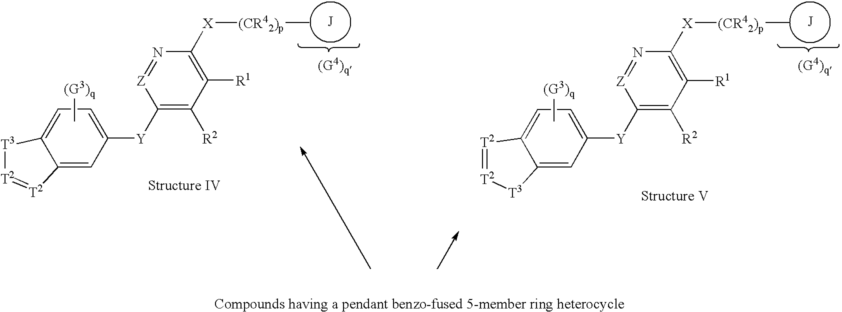
wherein R¹ and R²:
i) independently represent H or lower alky;
ii) together form a bridge of structure 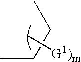
wherein binding is achieved via the terminal carbon atoms;
iii) together form a bridge of structure 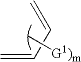
wherein binding is achieved via the terminal carbon atoms;
iv) together form a bridge of structure 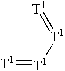
wherein one or two ring members T¹ are N and the others are CH or CG¹, and binding is achieved via the terminal atoms; or
v) together form a bridge containing two T² moieties and one T³ moiety, said bridge, taken together with the ring to which it is attached, forming a bicyclic of structure 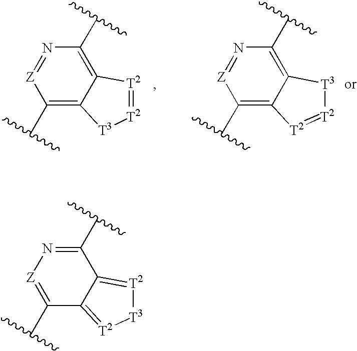
wherein
   each T² independently represents N, CH, or CG¹; and
   T³ represents S, O, CR⁴G¹, C(R⁴)₂, or NR³;

G¹ is a substituent independently selected from the group consisting of —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; —OCO₂R³; optionally substituted heteroarylalkyl; optionally substituted heteroaryloxy; —S(O)ₚ(optionally substituted heteroaryl); optionally substituted heteroarylalkyloxy; —S(O)ₚ(optionally substituted heteroarylalkyl); —CHO; —OCON(R⁶)₂; —NR³CO₂R⁶; —NR³CON(R⁶)₂.

R³ is H or lower alkyl.

R⁶ is independently selected from the group consisting of H; alkyl; cycloalkyl; optionally substituted aryl; optionally substituted aryl lower alkyl; lower alkyl-N(R³)₂; and lower alkyl-OH.

R⁴ is H, halogen, or lower alkyl.

p is 0, 1, or 2.

X is selected from the group consisting of O, S, and NR³.

Y is selected from the group consisting of lower alkylene; —CH₂—O—; —CH₂—S—; —CH₂—NH—; —O—; —S—; —NH—; —(CR⁴₂)ₙ—S(O)ₚ-(5-membered heteroaryl)-(CR⁴₂)ₛ—; —(CR⁴₂)ₙ—C(G²)(R⁴)—(CR⁴₂)ₛ; wherein n and s are each independently 0 or an integer of 1-2; and G² is selected from the group consisting of —CN, —CO₂R³, —CON(R⁶)₂, and —CH₂N(R⁶)₂; —O—CH₂—; —S(O)—; —S(O)₂—; —SCH₂—; —S(O)CH₂—; —S(O)₂CH₂—; —CH₂S(O)—; and —CH₂S(O)₂—.

Z is CH, —CG³, or N.

q is 0 or 1;

G³ is a monovalent moiety selected from the group consisting of lower alkyl; —NR³COR⁶; carboxy-substituted alkyl; lower alkoxycarbonyl-substituted alkyl; —OR⁶; —SR⁶; —S(O)R⁶; —S(O)₂R⁶; —OCOR⁶; —COR⁶; —CO₂R⁶; —CH₂OR³; —CON(R⁶)₂; —S(O)₂N(R⁶)₂; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy, —S(O)ₚ(optionally substituted heteroaryl); optionally substituted heteroarylalkyloxy; —S(O)ₚ(optionally substituted heteroarylalkyl); —OCON(R⁶)₂; —NR³CO₂R⁶; and —NR³CON(R⁶)₂;

J is a ring selected from the group consisting of aryl; pyridyl; and cycloalkyl.

q′ represents the number of substituents G⁴ on ring J and is 0, 1, 2, 3, 4, or 5.

G⁴ is a monovalent or bivalent moiety selected from the group consisting of —N(R⁶)₂; —NR³COR⁶; halogen; alkyl; cycloalkyl; lower alkenyl; lower cycloalkenyl; halogen-substituted alkyl; amino-substituted alkyl; N-lower alkylamino-substituted alkyl; N,N-di-lower alkylamino-substituted alkyl; N-lower alkanoylamino-substituted alkyl; hydroxy-substituted alkyl; cyano-substituted alkyl; carboxy-substituted alkyl; lower alkoxycarbonyl-substituted alkyl; phenyl lower alkoxycarbonyl-substituted alkyl; halogen-substituted alkylamino; amino-substituted alkylamino; N-lower alkylamino-substituted alkylamino; N,N-di-lower alkylamino-substituted alkylamino; N-lower alkanoylamino-substituted alkylamino; hydroxy-substituted alkylamino; cyano-substituted alkylamino; carboxy-substituted alkylamino; lower alkoxycarbonyl-substituted alkylamino; phenyl-lower alkoxycarbonyl-substituted alkylamino; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —CH₂OR³; —NO₂; —CN; amidino; guanidino; sulfo; —B(OH)₂; optionally substituted aryl; optionally substituted heteroaryl; optionally substituted saturated heterocyclyl; optionally substituted partially unsaturated heterocyclyl; —OCO₂R³; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —CHO; —OCON(R⁶)₂; —NR³CO₂R⁶; —NR³CON(R⁶)₂; and fused ring-forming bivalent bridges attached to and connecting adjacent positions of ring J, said bridges having the structures: 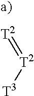
wherein
   each T² independently represents N, CH, or CG^{4′};
   T³ represents S, O, CR⁴G^{4′}, C(R⁴)₂, or NR³; wherein
      G4′ represents any of the above-defined moieties G⁴ which are monovalent; and
   binding to ring J is achieved via terminal atoms T² and T³; 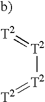
wherein
   each T² independently represents N, CH, or CG^{4′}; wherein
      G4′ represents any of the above-defined moieties G⁴ which are monovalent; and
   with the proviso that a maximum of two bridge atoms T² may be N; and
   binding to ring J is achieved via terminal atoms T²; and 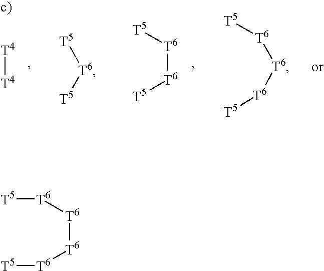
wherein
   each T⁴, T⁵, and T⁶ independently represents O, S, CR⁴G^{4′}, C(R⁴)₂, or NR³; wherein
      G4′ represents any of the above-defined moieties G⁴ which are monovalent; and
   binding to ring J is achieved via terminal atoms T⁴ or T⁵;
   with the provisos that:
      i) when one T⁴ is O, S, or NR³, the other T⁴ is CR⁴G^{4′} or C(R⁴)₂;
      ii) a bridge comprising T⁵ and T⁶ atoms may contain a maximum of two heteroatoms O, S, or N; and
      iii) in a bridge comprising T⁵ and T⁶ atoms, when one T⁵ group and one T⁶ group are O atoms, or two T⁶ groups are O atoms, said O atoms are separated by at least one carbon atom.

When G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members; and with the further provisos that: in G¹, G², and G⁴, when two groups R³ or R⁶ are each alkyl and located on the same N atom they may be linked by a bond, an O, an S, or NR³ to form a N-containing heterocycle of 5-7 ring atoms.

When an aryl, heteroaryl, or heterocyclyl ring is optionally substituted, that ring may bear up to 5 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂R³, —CHO, —CH₂OR³, —OCO₂R³, —CON(R⁶)₂, —OCON(R⁶)₂, —NR³CON(R⁶)₂, nitro, amidino, guanidino, mercapto, sulfo, and cyano.

When any alkyl group is attached to O, S, or N, and bears a hydroxyl substituent, then said hydroxyl substituent is separated by at least two carbon atoms from the O, S, or N to which the alkyl group is attached.

In another embodiment the method comprises compounds having structural formula III, IV, or V and the accompanying definitions wherein p is 0; j is phenyl or cycloalkyl; and R¹ and R²
i) together form a bridge containing two T² moieties and one T³ moiety, said bridge, taken together with the ring to which it is attached, forming a bicyclic of structure 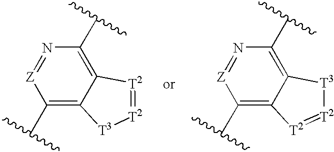
wherein
   each T² independently represents N, CH, or CG¹; and
   T³ represents S, O, CH₂, or NR³;
   with the proviso that when T³ is O or S, at least one T² is CH or CG¹; or
ii) together form a bridge of structure 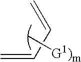
wherein binding is achieved via the terminal carbon atoms; or
iii) together form a bridge of structure 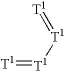
wherein one or two ring members T¹ are N and the others are CH or CG¹, and binding is achieved via the terminal atoms.

In a further embodiment the method comprises a compound of structural formula III, IV, or V and the accompanying definitions wherein Y is selected from a group consisting of lower alkylene; —CH₂—O—; —CH₂—S—; —CH₂—NH—; —O—; —S—; and —NH—.

G1 is selected from a group consisting of —N(R⁶)₂; alkyl; amino-substituted alkyl; N-lower alkylamino-substituted alkyl; N,N-di-lower alkylamino-substituted alkyl; hydroxy-substituted alkyl; carboxy-substituted alkyl; amino-substituted alkylamino; N-lower alkylamino-substituted alkylamino; N,N-di-lower alkylamino-substituted alkylamino; hydroxy-substituted alkylamino; carboxy-substituted alkylamino; —OR⁶; —S(O)R⁶; —S(O)₂R⁶; —OCOR⁶; —COR⁶; —CO₂R⁶; —CON(R⁶)₂; —CH₂OR³; —NO₂; —CN; amidino; guanidino; sulfo; optionally substituted heteroaryl; optionally substituted saturated heterocyclyl; optionally substituted saturated heterocyclylalkyl; optionally substituted partially unsaturated heterocyclyl; optionally substituted partially unsaturated heterocyclylalkyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; and —NR³CON(R⁶)₂.

G³ is selected from a group consisting of OH, lower alkyl, and O-lower alkyl.

G⁴ is selected from a group consisting of halogen; alkyl; cycloalkyl; lower alkenyl; lower cycloalkenyl; halogen-substituted alkyl; hydroxy-substituted alkyl; cyano-substituted alkyl; lower alkoxycarbonyl-substituted alkyl; phenyl lower alkoxycarbonyl-substituted alkyl; —OR⁶; —SR⁶; —S(O)R⁶; —S(O)₂R⁶; halogenated lower alkoxy, halogenated lower alkylthio; halogenated lower alkylsulfonyl; —OCOR⁶; —COR⁶; —CO₂R⁶; —CON(R⁶)₂; —CH₂OR³; —NO₂; —CN; optionally substituted aryl; optionally substituted heteroaryl; optionally substituted saturated heterocyclyl; optionally substituted partially unsaturated heterocyclyl; optionally substituted heteroarylalkyl; optionally substituted heteroaryloxy, —S(O)ₚ(optionally substituted heteroaryl); optionally substituted heteroarylalkyloxy, —S(O)ₚ(optionally substituted heteroarylalkyl); —OCON(R⁶)₂; —NR³CO₂R⁶; —NR³CON(R⁶)₂; and fused ring-forming bivalent bridges attached to and connecting adjacent positions of ring J, said bridges having the structures: 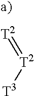
wherein
   each T² independently represents N, CH, or CG^{4′};
   T³ represents S, O, CR⁴G^{4′}, C(R⁴)₂, or NR³; wherein
      G4′ represents any of the above-defined moieties G⁴ which are monovalent; and
   binding to ring J is achieved via terminal atoms T² and T³; 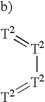
wherein
   each T² independently represents N, CH, or CG^{4′}; wherein
      G4′ represents any of the above-defined moieties G⁴ which are monovalent; and
   with the proviso that a maximum of two bridge atoms T² may be N; and
   binding to ring J is achieved via terminal atoms T²; and 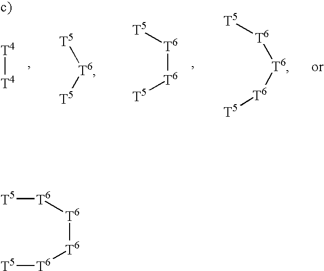
wherein
   each T⁴, T⁵, and independently represents O, S, CR⁴G^{4′}, C(R⁴)₂, or NR³; wherein
      G4′ represents any of the above-defined moieties G⁴ which are monovalent; and
   binding to ring 3 is achieved via terminal atoms T⁴ or T⁵;
   with the provisos that:
      i) when ones is O, S, or NR³, the other T⁴ is CR⁴G^{4′} or C(R⁴)₂;
      ii) a bridge comprising T⁵ and T⁶ atoms may contain a maximum of two heteroatoms O, S, or N; and
      iii) in a bridge comprising T⁵ and T⁶ atoms, when one T⁵ group and one T⁶ group are O atoms, or two T⁶ groups are O atoms, said O atoms are separated by at least one carbon atom;

When G⁴ is an alkyl group located on ring 3 adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members.

In a preferred embodiment of the invention the method comprises a compound of structural formula III, wherein one R⁶ is H, and one R⁶ is methyl; Y is —CH₂—O—; Z is N; X is NH; P is 0, J is a phenyl ring; q′ is 1, G⁴ is 4-Cl; and R¹ and R² together form a bridge containing two T² moieties and one T³ moiety, said bridge, taken together with the ring to which it is attached, forming a bicyclic of structure 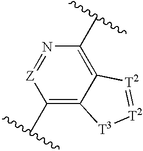
wherein T³ is O, and T² is CH.

Preferred compounds of the invention have an IC₅₀ of at least about 1 μM, more preferably at least about 2 μM, at least about 5 μM, at least about 10 μM, and most preferably at least about 15 μM, 20 μM, 25 μM or 30 μM with one or more P450 isoenzyme, such as Cyp1A2, CYP2C9, CYP2C19, CYP2D6 and CYP3A4.

Thus, in one aspect, the present invention provides the use of compounds with amidated pendant pyridine rings, e.g., as described herein, which are inhibitors of KDR but are weak inhibitors of P450 isoenzymes, in combination therapy with various other chemotherapeutic agents for the treatment of hyperproliferative diseases and/or diseases associated with angiogenesis, e.g., cancer.

In another aspect, the present invention provides the use of compounds with core heterocycles but lacking pendant pyridine rings, e.g., as described herein, which are inhibitors of KDR but are weak inhibitors of P450 isoenzymes in combination therapy with various other chemotherapeutic agents for the treatment of hyperproliferative diseases and/or diseases associated with angiogenesis, e.g., cancer.

Preferred compounds which have low P450 inhibitory activity are set forth in Table 1 (see Examples section below). Other preferred compounds are set forth in Tables 2-4. The example numbers given in column 1 of Tables 2 and 4 below are the same as the example numbers given for the same compounds in WO 01/23375. The example numbers given in column 1 of Table 3 are the same as the example numbers given for the same compounds in either WO 01/23375 or WO 01/10859 as indicated in Table 3.

TABLE 2
Compounds described herein and in WO 01/23375 that are expected to have low
P450 inhibition activity

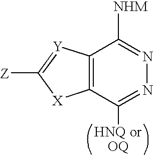

| Ex. # | X | Y | Z | MNH | NHO or OQ |
|---|---|---|---|---|---|
| 15 | O | CH | CONHCH₃ | 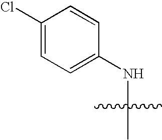 | 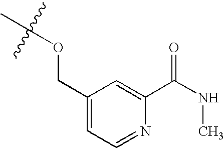 |
| 18 | S | CH | H | 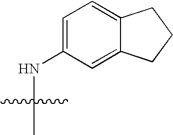 | 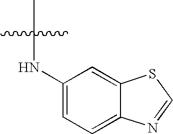 |
| 22 | O | CH | H | 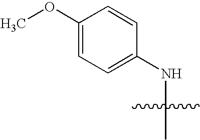 | 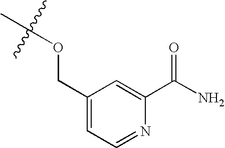 |
| 26 | S | CH | H | 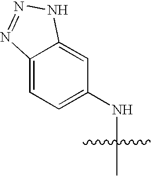 | 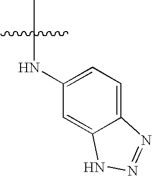 |
| 27 | S | CH | H | 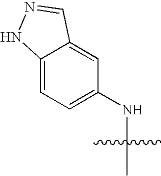 | 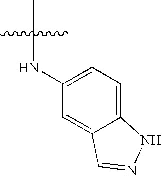 |
| 28 | O | CH | H | 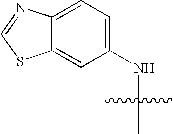 | 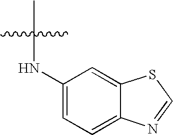 |
| 29 | O | CH | H | 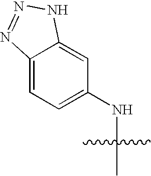 | 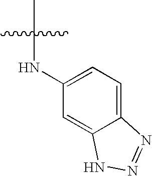 |
| 30 | O | CH | H | 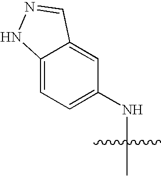 | 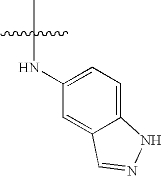 |
| 31 | N | N | H | 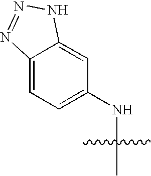 | 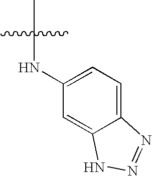 |
| 32 | N | N | H | 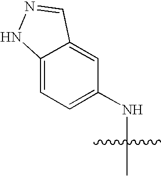 | 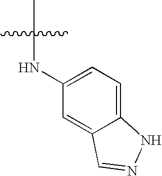 |
| 33 | N | N | H | 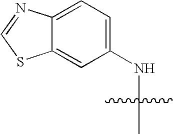 | 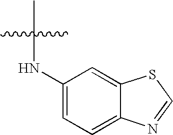 |
| 35 | O | CH | H | 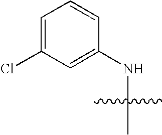 | 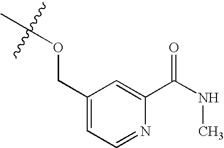 |
| 36 | O | CH | H | 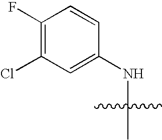 | 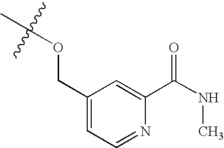 |
| 38 | O | CH | H | 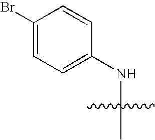 | 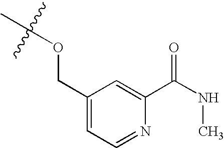 |
| 39 | O | CH | H | 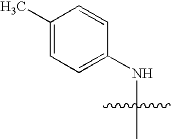 | 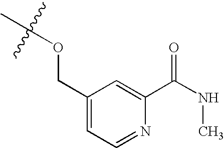 |
| 40 | O | CH | H | 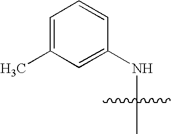 | 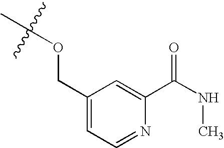 |
| 41 | O | CH | H | 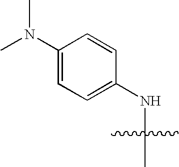 | 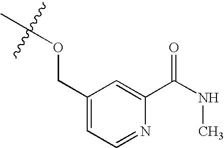 |
| 42 | O | CH | H | 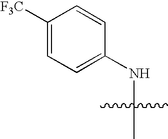 | 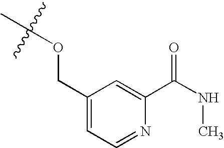 |
| 43 | O | CH | H | 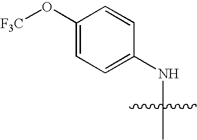 | 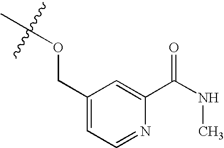 |
| 44 | O | CH | H | 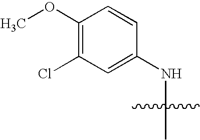 | 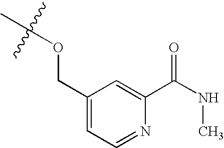 |
| 45 | O | CH | H | 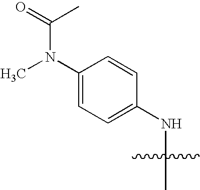 | 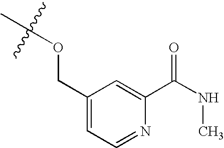 |
| 46 | O | CH | H | 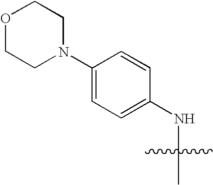 | 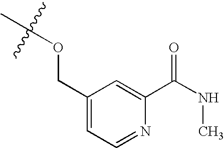 |
| 47 | O | CH | H | 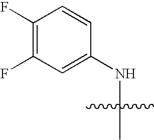 | 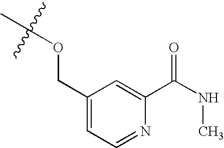 |
| 48 | O | CH | H | 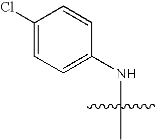 | 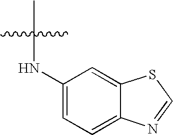 |
| 50 | O | CH | H | 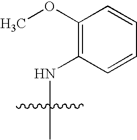 | 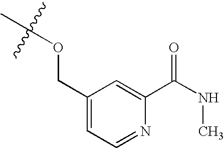 |
| 51 | O | CH | H | 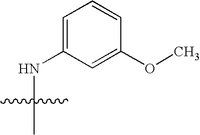 | 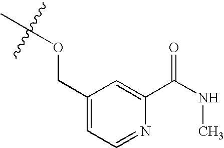 |
| 52 | O | CH | H | 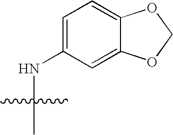 | 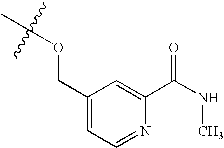 |
| 53 | O | CH | H | 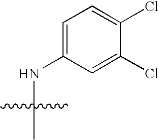 | 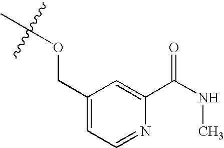 |
| 54 | O | CH | H | 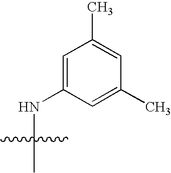 | 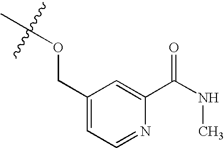 |
| 55 | O | CH | H | 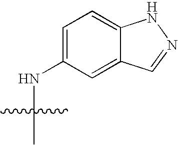 | 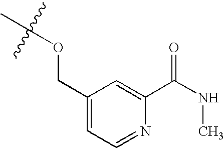 |
| 57 | O | CH | H | 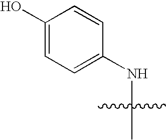 | 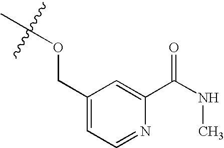 |
| 59 | O | CH | H | 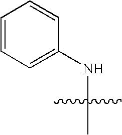 | 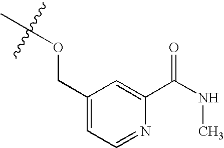 |
| 60 | O | CH | H | 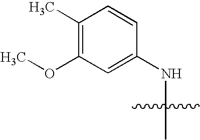 | 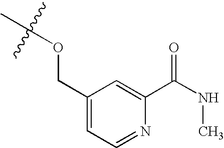 |
| 61 | O | CH | H | 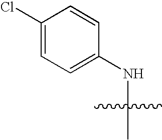 | 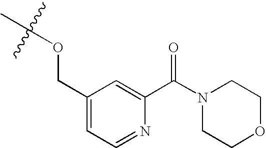 |
| 62 | O | CH | H | 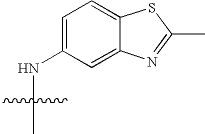 | 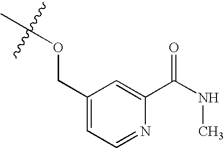 |
| 63^{c} | O | CH | H | 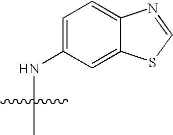 | 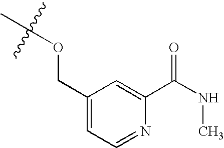 |
| 66 | S | CH | H | 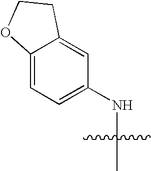 | 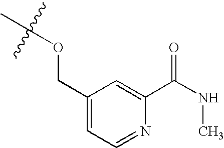 |
| 67 | S | CH | H | 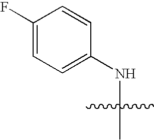 | 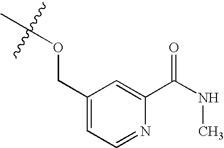 |
| 69 | S | CH | H | 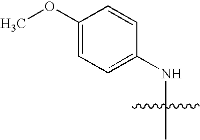 | 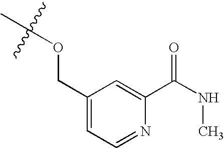 |
| 70 | S | CH | H | 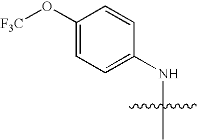 | 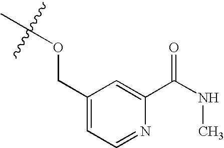 |
| 71 | S | CH | H | 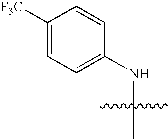 | 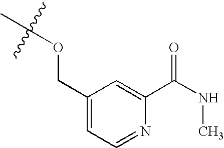 |
| 72 | S | CH | H | 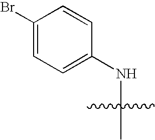 | 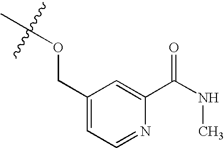 |
| 73 | S | CH | H | 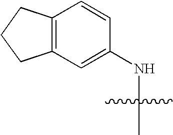 | 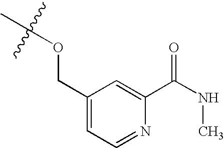 |
| 74 | S | CH | H | 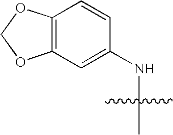 | 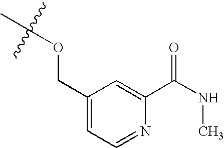 |
| 75 | S | CH | H | 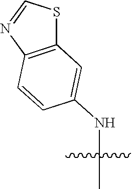 | 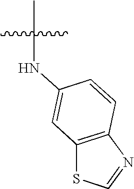 |
| 76 | S | CH | H | 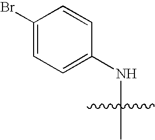 | 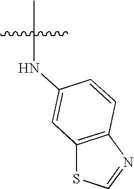 |
| 77 | S | CH | H | 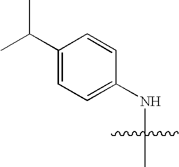 | 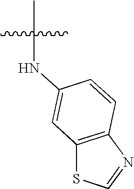 |
| 78 | S | CH | H | 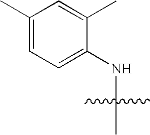 | 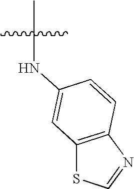 |
| 79 | S | CH | H | 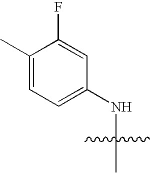 | 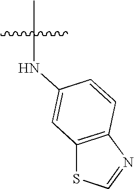 |
| 80 | S | CH | H | 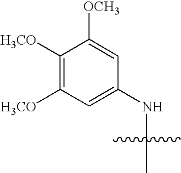 | 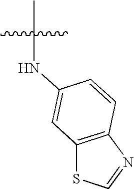 |
| 82A | O | CH | H | 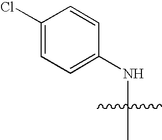 | 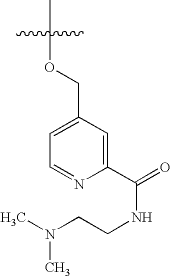 |
| 82C | O | CH | H | 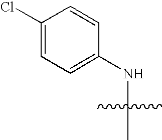 | 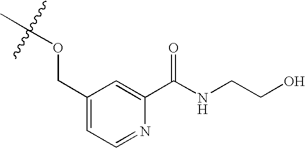 |
| 82D | O | CH | H | 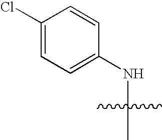 | 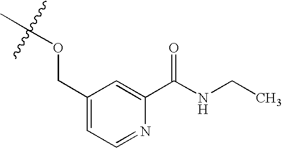 |

“Ex. #” refers to the number of the Example in WO 01/23375 that describes the compound listed in the Table.

TABLE 3
Compounds described in WO 01/23375 or in WO 01/10859 that are expected to have
low P450 inhibition activity

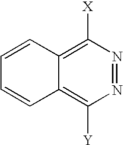

| Example # | X | Y |
|---|---|---|
| 6 WO 0110859 | 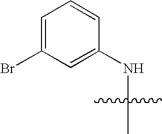 | 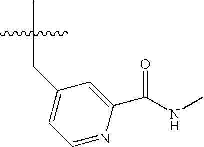 |
| 7 WO 0110859 | 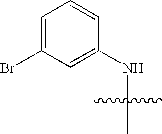 | 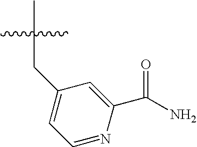 |
| 11 WO 0110859 HCl salt | 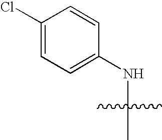 | 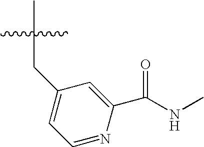 |
| 12 WO 0110859 Mesylate salt | 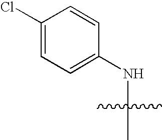 | 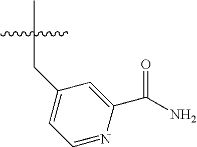 |
| 13 WO 0110859 HCl salt | 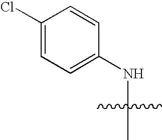 | 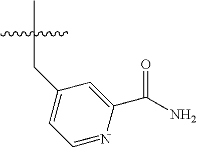 |
| 14 WO 0110859 Mesylate salt | 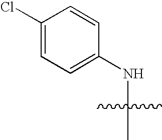 | 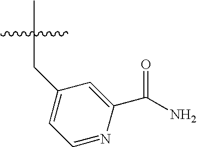 |
| 15 WO 0110859 HCl salt | 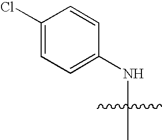 | 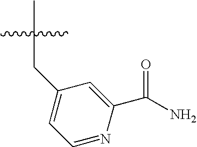 |
| 16 WO 0110859 Mesylate salt | 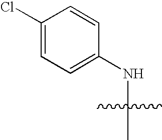 | 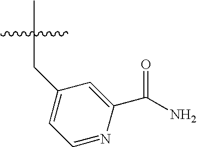 |
| 93 WO 0123375 | 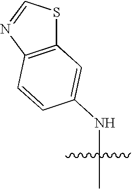 | 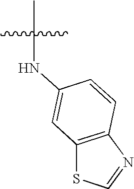 |
| 94 WO 0123375 | 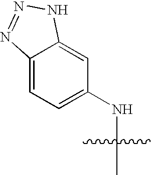 | 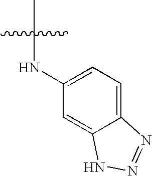 |
| 95 WO 0123375 | 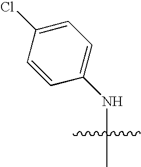 | 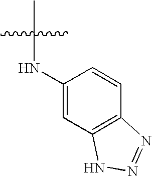 |
| 96 WO 0123375 | 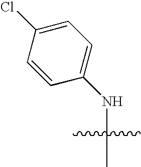 | 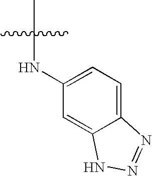 |
| 97 WO 0123375 | 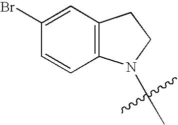 | 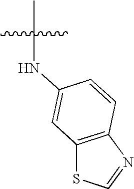 |
| 98 WO 0123375 | 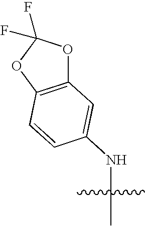 | 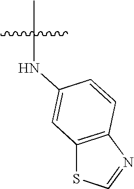 |
| 99 WO 0123375 | 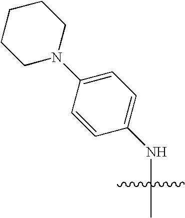 | 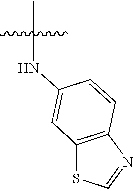 |
| 100 WO 0123375 | 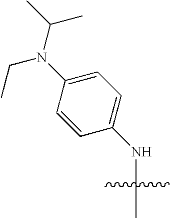 | 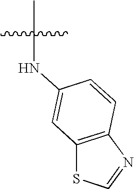 |
| 101 WO 0123375 | 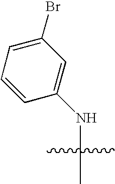 | 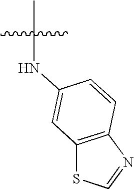 |
| 102 WO 0123375 | 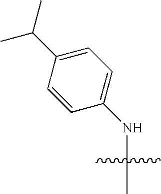 | 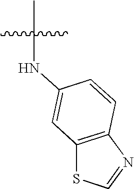 |
| 103 WO 0123375 | 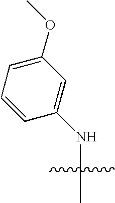 | 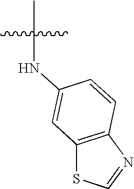 |
| 104 WO 0123375 | 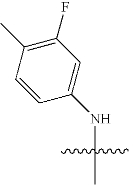 | 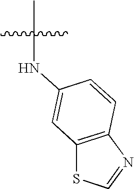 |
| 105 WO 0123375 | 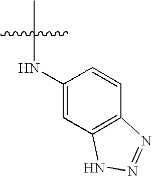 | 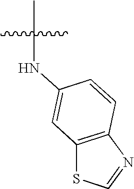 |

“Example #” refers to the number of the Example in WO 01/23375 or in WO 01/10859 that describes the compound listed in the Table.

TABLE 4
Salts described in WO01/23375 that are expected to have low P450
inhibition activity

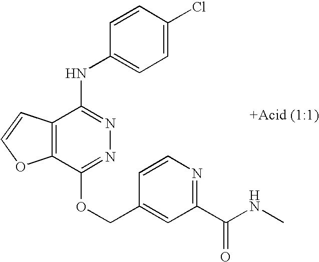

| Example # | Acid Used |
|---|---|
| 106 | 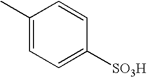 |
| 107 | 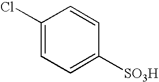 |
| 108 | 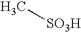 |
| 109 | 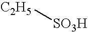 |
| 110 | (HCl)₂* |
| 111 | HBr |
| 112 | H₂SO₄ |
| 113 | HNO₃ |
| 114 | 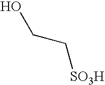 |
| 115 | 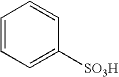 |

*2:1 salt was obtained with HCl
“Example #” refers to the number of the Example in WO 01/23375 that describes the compound listed in the Table.

In another embodiment the method of the invention comprises administering the first chemotherapeutic agent and the compound of general structural formula I simultaneously.

In another embodiment, the first chemotherapeutic agent and the compound of general structural formula I are administered sequentially.

In another embodiment of the invention the subject is human.

In another embodiment the subject is a non-human mammal.

General Preparative Methods

The compounds of the invention may be prepared by use of known chemical reactions and procedures. For example, substituted pyridazines, fused pyridazines and substituted pyridines can be prepared as described in WO 01/10859 and WO 01/23375, and pending U.S. patent application Ser. Nos. 09/371,322 and 09/407,600, under “General Preparative Methods” and in the Examples, all of which are specifically incorporated by reference herein.

Pharmaceutical Compositions

In one embodiment, the invention provides a method for treating a subject having a proliferative disease or a disease associated with angiogenesis and/or hyperpermeability, comprising administering to the subject a therapeutically effective amount of a pharmaceutical composition comprising a first chemotherapeutic agent and a therapeutically effective amount of a pharmaceutical composition comprising a compound of the invention. In one embodiment, the compound of the invention and the first chemotherapeutic compound are different from each other. The compound of the invention and the chemotherapeutic compound can be administered simultaneously, or sequentially. They can be administered by the same route or by different routes. For example, one of the two compounds can be administered orally and the other agent can be administered by injection. The pharmaceutical compositions and methods of administrations described below apply to the compound of the invention as well as to the chemotherapeutic compound.

Prodrugs of the compounds of the invention and/or chemotherapeutic drugs can also be administered. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995)). Commonly used prodrugs of the disclosed oxazolyl-phenyl-2,4-diamino-pyrimidine compounds can be designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996)).

Accordingly, the invention provides pharmaceutical compositions comprising one or more of the compounds of the invention, or their salts or prodrugs forms thereof, with a pharmaceutically acceptable ingredient.

The compounds may be administered orally, dermally, parenterally, by injection, by inhalation or spray, or sublingually, rectally or vaginally in dosage unit formulations. The term ‘administered by injection’ includes intravenous, intraarticular, intramuscular, subcutaneous and parenteral injections, as well as use of infusion techniques. Dermal administration may include topical application or transdermal administration. One or more compounds may be present in association with one or more non-toxic pharmaceutically acceptable carriers and if desired, other active ingredients.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations.

Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid. Pharmaceutical compositions of the invention may also be in the form of oil-in-water emulsions. The oil phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal or vaginal temperature and will therefore melt in the rectum or vagina to release the drug. Such materials include cocoa butter and polyethylene glycols.

Compounds of the invention may also be administered transdermally using methods known to those skilled in the art (see, for example: Chien; “Transdermal Controlled Systemic Medications”; Marcel Dekker, Inc.; 1987. Lipp et al. WO 94/04157 3 Mar. 1994). For example, a solution or suspension of a compound of any of formulas I-V in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of any of formulas I-V may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures one or more materials selected from lower alcohols, lower ketones, lower carboxylic acid esters, polar ethers, lower hydrocarbons, halogenated hydrocarbons.

Suitable penetration enhancing materials for transdermal delivery systems are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl isobutyl tert-butyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated dicarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrene-butadiene coploymers, and natural and synthetic rubbers. Cellulose ethers, derivatized polyethylenes, and silicates may also be used as matrix components. Additional additives, such as viscous resins or oils may be added to increase the viscosity of the matrix.

For all regimens of use disclosed herein for compounds of any of formulas I-V, the daily oral dosage regimen will preferably be from 0.01 to 200 mg/Kg of total body weight. The daily dosage for administration by injection, including intravenous, intramuscular, subcutaneous and parenteral injections, and use of infusion techniques will preferably be from 0.01 to 200 mg/Kg of total body weight. The daily rectal dosage regimen will preferably be from 0.01 to 200 mg/Kg of total body weight. The daily vaginal dosage regimen will preferably be from 0.01 to 200 mg/Kg of total body weight. The daily topical dosage regimen will preferably be from 0.1 to 200 mg administered between one to four times daily. The transdermal concentration will preferably be that required to maintain a daily dose of from 0.01 to 200 mg/Kg. The daily inhalation dosage regimen will preferably be from 0.01 to 10 mg/Kg of total body weight.

It will be appreciated by those skilled in the art that the particular method of administration will depend on a variety of factors, all of which are considered routinely when administering therapeutics. It will also be understood, however, that the specific dose level for any given patient will depend upon a variety of factors, including, but not limited to the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of a compound of any of formulas I-V or a pharmaceutically acceptable salt thereof given for a defined number of days, can be ascertained by those skilled in the art using conventional treatment tests.

Biological Assays

Set forth below are assays for confirming the biological activities of compounds of the invention.

KDR Assay

Set forth below is an exemplary in vitro assay that can be used for determining the efficiency of compounds to modulate, in particular inhibit, autophosphorylation of the KDR receptor kinase domain.

The cytosolic kinase domain of KDR kinase is expressed as a 6His fusion protein in Sf9 insect cells. The KDR kinase domain fusion protein is purified over a Ni⁺⁺ chelating column. Ninety-six well ELISA plates are coated with 5 μg poly(Glu4;Tyr1) (Sigma Chemical Co., St Louis, Mo.) in 100 μl HEPES buffer (20 mM HEPES, pH 7.5, 150 mM NaCl, 0.02% Thimerosal) at 4° overnight. Before use, the plate is washed with HEPES, NaCl buffer and the plates are blocked with 1% BSA, 0.1% Tween 20 in HEPES, NaCl buffer.

Test compounds are serially diluted in 100% DMSO from 4 mM to 0.12 μM in half-log dilutions. These dilutions are further diluted twenty fold in H₂O to obtain compound solutions in 5% DMSO. Following loading of the assay plate with 85 μl of assay buffer (20 mM HEPES, pH 7.5, 100 mM KCl, 10 mM MgCl₂, 3 mM MnCl₂, 0.05% glycerol, 0.005% Triton X-100, 1 mM-mercaptoethanol, with or without 3.3 M ATP), 5 μl of the diluted compounds are added to bring the assay volume to 90 μl. The assay is initiated by the addition of 10 μl (30 ng) of KDR kinase domain. Final concentrations are between 10 μM, and 0.3 nM in 0.25% DMSO.

The assay plate is incubated with test compound or vehicle alone with gentle agitation at room temperature for 60 minutes. The wells are washed and phosphotyrosines (PY) are probed with an anti-phosphotyrosine (PY), mAb clone 4G10 (Upstate Biotechnology, Lake Placid, N.Y.). PY/anti-PY complexes are detected with an anti-mouse IgG/HRP conjugate (Amersham International plc, Buckinghamshire, England). Phosphotyrosine is quantitated by incubation with 100 μl 3, 3′, 5, 5′ tetramethylbenzidine solution (Kirkegaard and Perry, TMB Microwell 1 Component peroxidase substrate). Color development is arrested by the addition of 100 μl 1% HCl-based stop solution (Kirkegaard and Perry, TMB 1 Component Stop Solution).

Optical densities are determined spectrophotometrically at 450 nm in a 96-well plate reader, SpectraMax 250 (Molecular Devices). Background (no ATP in assay) OD values are subtracted from all ODs and the percent inhibition is calculated according to the equation:

$$\% Inhibition = \frac{( OD ( vehicle control ) - OD ( with compound ) ) \times 100 }{OD ( vehicle control ) - OD ( no ATP added )}$$

The IC₅₀ values are determined with a least squares analysis program using compound concentration versus percent inhibition. Preferred compounds of the invention have an IC₅₀ value of at most about 10 μM, preferably less than about 1 μM, even more preferably less than about 100 nM in this assay. IC₅₀ values for the compounds of the invention determined in the above-described assay are provided in WO 01/10859 and WO 01/23375 and in pending U.S. application Ser. Nos. 09/407,600 and 09/371,322, under “KDR Assay.”

Cell Mechanistic Assay-Inhibition of 3T3 KDR Phosphorylation

Set forth below is an exemplary cell-based assay that can be used for determining the efficiency of compounds to modulate, in particular inhibit, KDR receptor activation.

NIH3T3 cells expressing the full length KDR receptor are grown in DMEM (Life Technologies, Inc., Grand Island, N.Y.) supplemented with 10% newborn calf serum, low glucose, 25 mM/L sodium pyruvate, pyridoxine hydrochloride and 0.2 mg/ml of G418 (Life Technologies Inc., Grand Island, N.Y.). The cells are maintained in collagen I-coated T75 flasks (Becton Dickinson Labware, Bedford, Mass.) in a humidified 5% CO₂ atmosphere at 37° C.

Fifteen thousand cells are plated into each well of a collagen I-coated 96-well plate in the DMEM growth medium. Six hours later, the cells are washed and the medium is replaced with DMEM without serum. After overnight culture to quiesce the cells, the medium is replaced by Dulbecco's phosphate-buffered saline (Life Technologies Inc., Grand Island, N.Y.) with 0.1% bovine albumin (Sigma Chemical Co., St Louis, Mo.). After adding various concentrations (0-300 nM) of test compounds to the cells in 1% final concentration of DMSO, the cells are incubated at room temperature for 30 minutes. The cells are then treated with VEGF (30 ng/ml) for 10 minutes at room temperature. Following VEGF stimulation, the buffer is removed and the cells are lysed by addition of 150 μl of extraction buffer (50 mM Tris, pH 7.8, supplemented with 10% glycerol, 56 mM BGP, 2 mM EDTA, 10 mM NaF, 0.5 mM NaVO4, and 0.3% TX-100) at 4° C. for 30 minutes.

To assess receptor phosphorylation, 100 microliters of each cell lysate is added to the wells of an ELISA plate precoated with 300 ng of antibody C20 (Santa Cruz Biotechnology, Inc., Santa Cruz, Calif.). Following a 60-minute incubation, the plate is washed and bound KDR is probed for phosphotyrosine using an anti-phosphotyrosine mAb clone 4G10 (Upstate Biotechnology, Lake Placid, N.Y.). The plate is washed and wells are incubated with anti-mouse IgG/HRP conjugate (Amersham International plc, Buckinghamshire, England) for 60 minutes. Wells are washed and phosphotyrosine was quantitated by addition of 100 μl per well of 3,3′,5,5′ tetramethylbenzidine (Kirkegaard and Perry, TMB Microwell 1 Component peroxidase substrate) solution. Color development is arrested by the addition of 100 μl 1% HCl based stop solution (Kirkegaard and Perry, TMB 1 Component Stop Solution).

Optical densities (OD) are determined spectrophotometrically at 450 nm in a 96-well plate reader (SpectraMax 250, Molecular Devices). Background (no VEGF added) OD values are subtracted from all ODs and percent inhibition was calculated according to the equation:

$$\% Inhibition = \frac{\begin{matrix}( OD ( VEGF control ) - \\ OD ( with test compound ) ) \times 100\end{matrix}}{OD ( VEGF control ) - OD ( no VEGF added )}$$

IC₅₀s are determined with a least squares analysis program using compound concentration versus percent inhibition. Preferred compounds of the invention have an IC₅₀ value of at most about 10 μM, preferably at most about 1 μM, even more preferably at most about 100 nM, 10 nM or 1 nM, in this assay. IC₅₀ values for the compounds of the invention determined in the above-described assay are provided in WO 01/10859 and WO 01/23375 and in pending U.S. application Ser. Nos. 09/407,600 and 09/371,322, under “Cell mechanistic assay-Inhibition of 3T3 KDR phosphorylation.”

Matrigel® Angiogenesis Model

Set forth below is an exemplary in vivo assay for determining the effectiveness of compounds to modulate, in particular inhibit, angiogenesis.

Preparation of Matrigel Plugs and in vivo Phase: Matrigel® (Collaborative Biomedical Products, Bedford, Mass.) is a basement membrane extract from a murine tumor composed primarily of laminin, collagen IV and heparan sulfate proteoglycan. It is provided as a sterile liquid at 4° C., but rapidly forms a solid gel at 37° C.

Liquid Matrigel at 4° C. is mixed with SK-MEL2 human tumor cells that are transfected with a plasmid containing the murine VEGF₁₆₅ gene with a selectable marker. Tumor cells are grown in vitro under selection and cells are mixed with cold liquid Matrigel at a ratio of 2×10⁶ per 0.5 ml. One half milliliter is implanted subcutaneously near the abdominal midline using a 25 gauge needle. Test compounds are dosed as solutions in Ethanol/Cremaphor EL/saline (12.5%:12.5%:75%) at 30, 100, and 300 mg/kg po once daily starting on the day of implantation. Mice are euthanized 12 days post-implantation and the Matrigel pellets are harvested for analysis of hemoglobin content

Hemoglobin Assay: The Matrigel pellets are placed in 4 volumes (w/v) of 4° C. Lysis Buffer (20 mM Tris pH 7.5, 1 mM EGTA, 1 mM EDTA, 1% Triton X-100 [EM Science, Gibbstown, N.J.], and complete, EDTA-free protease inhibitor cocktail [Mannheim, Germany]), and homogenized at 4° C. Homogenates are incubated on ice for 30 minutes with shaking and centrifuged at 14K×g for 30 minutes at 4° C. Supernatants are transferred to chilled microfuge tubes and stored at 4° C. for hemoglobin assay.

Mouse hemoglobin (Sigma Chemical Co., St. Louis, Mo.) is suspended in autoclaved water (BioWhittaker, Inc, Walkersville, Md.) at 5 mg/ml. A standard curve is generated from 500 micrograms/ml to 30 micrograms/ml in Lysis Buffer (see above). Standard curve and lysate samples are added at 5 microliters/well in duplicate to a polystyrene 96-well plate. Using the Sigma Plasma Hemoglobin Kit (Sigma Chemical Co., St. Louis, Mo.), TMB substrate is reconstituted in 50 mls room temperature acetic acid solution. One hundred microliters of substrate is added to each well, followed by 100 microliters of Hydrogen Peroxide Solution to each well at room temperature. The plate is incubated at room temperature for 10 minutes.

Optical densities are determined spectrophotometrically at 600 nm in a 96-well plate reader, SpectraMax 250 Microplate Spectrophotometer System (Molecular Devices, Sunnyvale, Calif.). Background Lysis Buffer readings are subtracted from all wells.

Total sample hemoglobin content is calculated according to the following equation:
Total Hemoglobin=(Sample Lysate Volume)×(Hemoglobin Concentration)

The average Total Hemoglobin of Matrigel samples without cells is subtracted from each Total Hemoglobin Matrigel sample with cells. Percent inhibition is calculated according to the following equation:

$$\% Inhibition = \frac{\begin{matrix}( Average Total Hemoglobin \\ Drug \text{-} Treated Tumor Lysates ) \times 100\end{matrix}}{\begin{matrix}( Average Total Hemoglobin \\ Non \text{-} Treated Tumor Lysates )\end{matrix}}$$

Preferred compounds of the invention have an activity in this assay at 30, 100 and 300 mg/kg po sid with more than about 30%, preferably more than about 50% inhibition of total hemoglobin content of the Matrigel samples from the dosed animals vs. those from vehicle control animals. Values for the compounds of the invention determined in the above-described assay are provided in WO 01/10859 and WO 01/23375 and in pending U.S. application Ser. Nos. 09/407,600 and 09/371,322, under “Matrigel® Angiogenesis Model.”

P450 Assays

An exemplary assay for determining the P450 inhibiting activity of compounds is set forth in the Examples.

In Vivo Models for Testing the Efficacy of Compounds as Anti-Tumor Agents

Various in vivo animal models can be used for determining the efficacy of a therapy in preventing, inhibiting or eliminating hyperproliferative cells, such as those forming tumors, e.g., malignant tumors.

For example, in vivo evaluation of the tolerance and efficacy of combinations of compounds of the invention and paclitaxel can be conducted as follows using, e.g., the MDA-MB-231 human mammary tumor xenograft: Tumor cells (eg, MDA-MB 231 breast adenocarcinoma cell line; ATCC No. HTB 26; see also J. Natl. Cancer Inst. (Bethesda) 53, 661-74 [1974]) are grown in vitro using DMEM (GibcoBRL) supplemented with 10% FBS (Hyclone), 2 mM glutamine (GibcoBRL), 100 units/ml penicillin (GibcoBRL), and 100 μg/ml streptomycin (GibcoBRL) as media. Cells are subcultured twice per week using a 1:10 split ratio. Cells are harvested for inoculation into animals during mid-log phase growth at approximately 80% confluent cultures using a <5 min exposure to 0.25% trypsin-EDTA (GibcoBRL). The trypsin is then quenched by addition of media and a single cell suspension is generated by repeated pipetting. Cells are then pelleted at 1000 rpm for 5-8 min in a Beckman GPR centrifuge, and resuspended to a concentration of 1×10⁷ cells/ml in DMEM with no additives for inoculation into animals.

Tumors are initiated by implanting cells (5×10⁶ cells/animal) s.c. in the right flank of 6-8 week old female NCr nu/nu mice (Taconic Farms). Approximately 50% more mice than are actually intended for use in the study are initially implanted with tumor cells to allow for the selection of animals with a sufficiently small range of tumor sizes for inclusion in the study at the time treatment is initiated. When small but established and actively growing tumors are measurable, i.e. 75-125 mg tumor burden 10 days after implantation, treatment is initiated by the indicated route and schedule. Paclitaxel (Taxol®, Bristol-Myers-Squibb) is administered i.v. using a vehicle of 12.5% ethanol/12.5% cremophore/75% physiologic saline on a q15d x 2 schedule. Invention compound is administered p.o. using a vehicle of 95% PEG 400: 5% glycerin on a qd x 14 schedule starting the same day as the paclitaxel treatment. Tumor growth and animal body weights are monitored twice per week. Toxicity is assessed in terms of body weight loss and time to recovery of lost weight after cessation of treatment. Frank lethality is also recorded on a daily basis. Efficacy is assessed as tumor percent growth delay [%(T−C)/C], where T and C represent the median times for the tumors in the Treated and Control groups, respectively, to attain a size of 3 mass doublings from the size at the initiation of treatment. The individual animal's times to attain this evaluation size is statistically evaluated by the Kaplan-Meier estimate followed by the Mantel-Haenzel log-rank test. Significance is set at p<0.05.

As an alternative to cell line MDA-MB-231, other cell lines may also be used in the same manner, for example: the MCF-7 breast adenocarcinoma cell line (ATCC No. HTB 22; see also J. Natl. Cancer Inst. (Bethesda) 51, 1409-16 [1973]); the MDA-MB 468 breast adenocarcinoma cell line (ATCC No. HTB 132; see also In Vitro 14, 911-15 [1978]); the A-431 human epithelial cell line (American Type Culture Collection, Manassas, Va., USA, Catalogue Number ATCC CRL 1555); the Colo 205 colon carcinoma cell line (ATCC No. CCL 222; see also Cancer Res. 38, 1345-55 [1978]); the HCT 1 16 colon carcinoma cell line (ATCC No. CCL 247; see also Cancer Res. 41, 1751-6 [1981]); the DU145 prostate carcinoma cell line DU 145 (ATCC No. HTB 81; see also Cancer Res. 37, 4049-58 [1978]); and the PC-3 prostate carcinoma cell line PC-3 (ATCC No. CRL 1435; see also Cancer Res. 40, 524-34 [1980]).

Other animal models include animals which are transgenic for an oncogene and which develop tumors, e.g., carcinomas, that have genetic and pathological features that closely resemble human cancers. For example, in an MMTV-neu transgenic mouse lineage, 100% of the female mice develop mammary adenocarcinomas (Sacco et al., Gene Therapy 2:493497 (1995); Sacco et al., Gene Therapy 5:383-393 (1998)). Other animals transgenic for an oncogene are described in U.S. Pat. No. 5,925,803, by Leder et al. (Myc transgene); Muller et al. (1988) Cell 54:105 (Neu transgene); Weinstein et al. (2000) Mol. Med. 6:4 (Neu transgene); Kohl, et al., Nature Medicine, vol. 1, No. 8 (August 1995) (Ras transgene); U.S. Pat. No. 5,917,124 (SV40 TAg transgene).

Compounds of the invention can be studied together with other chemotherapeutic agents using similar protocols but substituting those agents for paclitaxel in the above procedure. Other agents should be dosed at efficacious yet non-toxic levels and in a manner consistent with published reports for those agents.

Chemotherapeutic Agents

Agents with which the compounds of the invention can be administered (together or sequentially) to a subject include any therapeutic compounds useful for treating diseases associated with abnormal angiogenesis and/or hypermeability processes, such as proliferative diseases.

In a preferred embodiment, a subject is treated with one or more compouns of the invention and one or more cytostatic or cytotoxic compound (or agent). The agent can be an inhibitor of polyamine biosynthesis, an inhibitor of protein kinase activity, e.g., an inhibitor of a serine/threonine or a tyrosine kinase, a cytokine, a negative growth regulator, or an aromatase inhibitor. Exemplary agents can be selected from a list which includes but are not limited to compounds listed on the cancer chemotherapy drug regiments in the 11^{th} Edition of the Merk Index, (1996), which is hereby incorporated by reference. Other anti-proliferative agents suitable for use with the composition of the invention include but are not limited to those compounds acknowledged to be used in the treatment of neoplastic diseases in Goodman and Gilman's The Pharmacological Basis of Therapeutics (Tenth Edition), editors J. G. Hartman, L. E. Limbird and A. G. Gilman, publ. by McGraw-Hill, pages 1381-1459, (2001).

A list of other chemotherapeutic agents includes but is not limited to compounds such as 2′,2′-difluorodeoxycytidine, 5-azacytidine, 5-fluorodeoxyuridine, 5-fluorouracil, 6-mercaptopurine, AG3340 and other MMP inhibitors, aminoglutethimide, angiostatin, asparaginase, azathioprine, bleomycin, busulfan, campothecin or related compounds that are topoisomerase I inhibitors, thalidomide, capecitabine, carboplatin, carmofur, carmustine, chlorambucil, cisplatin, cladribine, colaspase, COX-2 inhibitors, cyclophosphamide, cytarabine, dacarbazine, dactinomycin, daunorubicin, diethylstilbestrol, docetaxel, doxifluridine, doxorubicin (adriamycine) and liposomal doxorubicin, endostatin, epirubicin, epothilone, erythrohydroxynonyladenine, estramustine, ethinyl estradiol, etoposide, floxuridine, fludarabine phosphate, fluorouracil, fluoxymesterone, flutamide, gemcitabine, hexamethylmelamine, hydroxyprogesterone caproate, hydroxyurea, idarubicin, ifosfamide, interferon alpha and other interferons, irinotecan, L-asparaginase, leucovorin, leuprolide or peptide agents related to leuprolide, lomustine, mechlorethamine, medroxyprogesterone acetate, megestrol acetate, melphalan, mesna, methotrexate, metoxantrone, mitomycin C, mitotane, mitoxantrone, N-phosphonoacetyl-L aspartate (PALA), oxaliplatin, paclitaxel (taxol), pentostatin, plicamycin, polyglutamated taxanes, prednisolone, prednisone, procarbazine, raloxifen, semustine, streptozocin, tamoxifen or derivatives thereof or other “anti-estrogen” compounds, tegafur, teniposide, testosterone propionate, thioguanine, thiotepa, topotecan, trimethylmelamine, uracil-ftorafur (UFT), uridine, vinblastine, vincristine, vindesine and vinorelbine.

Exemplary Diseases

The combination therapies of the invention can be used for treating any disease associated with abnormal angiogenesis and/or hyperpermeability processes, such as those described in the Background of the Invention.

In a preferred embodiment, the disease is a proliferative disease, i.e., a disease associated with abnormal or excessive cell proliferation. Preferred diseases include those associated with benign tumors, malignant tumors or metastases. In a preferred embodiment, the combination therapies can be used for treating cancers.

The subject to be treated can be a mammal, e.g., a human, a canine, a feline, a bovine, an ovine, a porcine, and an equine.

Examples of cancers that can be treated include, but are not limited to, solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma. Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to, prostate and testicular cancer.

Tumors of the female reproductive organs include, but are not limited to, endometrial, cervical, ovarian, vaginal, and vulvar cancer, as well as sarcoma of the uterus.

Tumors of the digestive tract include, but are not limited to, anal, colon, colorectal, esophageal, gallblader, gastric, pancreatic, rectal, small-intestine, and salivary gland cancers.

Tumors of the urinary tract include, but are not limited to, bladder, penile, kidney, renal pelvis, ureter, and urethral cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal/hypopharyngeal/nasopharyngeal/oropharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

These disorders have been well characterized in man, but also exist with a similar etiology in other mammals, and can be treated by pharmaceutical compositions of the present invention.

Kits of the Invention

The invention further provides kits comprising one or more compounds of the invention. For example, compounds of the invention and/or materials and reagents required for administering the compounds of the invention may be assembled together in a kit. When the components of the kit are provided in one or more liquid solutions, the liquid solution preferably is an aqueous solution, with a sterile aqueous solution being particularly preferred.

The kit may further comprise one or more other drugs, e.g., a chemo- or radiotherapeutic agent. These normally will be a separate formulation, but may be formulated into a single pharmaceutically acceptable composition. The container means may itself be geared for administration, such as an inhalant, syringe, pipette, eye dropper, or other such like apparatus, from which the formulation may be applied to an infected area of the body, such as the lungs, or injected into an animal, or even applied to and mixed with the other components of the kit.

The compositions of these kits also may be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the agent and, e.g., explaining how the agent will decrease proliferation of cells.

The kits of the present invention also will typically include a means for containing the vials in close confinement for commercial sale such as, e.g., injection or blow-molded plastic containers into which the desired vials are retained. Irrespective of the number or type of containers, the kits of the invention may also comprise, or be packaged with a separate instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of reactions.

The present invention is further illustrated by the following examples which should not be construed as limiting in any way. The contents of all cited references (including literature references, issued patents, published patent applications and pending applications as cited throughout this application) are hereby expressly incorporated by reference.

### EXAMPLES

### Example 1

### Compounds Having Low P450 Inhibitory Activity

The ability of the compounds described herein to inhibit cytochrome P450 activity was determined in vitro using cDNA-expressed human cytochrome P450 enzymes (Supersomes) obtained from GENTEST Corporation and various fluorescent probe substrates. Fluorometric Cytochrome P450 Inhibition assays were conducted in 96 well micotitre plates as described by GENTEST Corporation, Woburn Mass. (www.gentest.com). Compounds were dissolved in 70% DMSO such that the concentration of DMSO in the assay was 0.7%. Varying concentrations of all test compounds (10 μM, 3 μM, 1 μM, 300 nM and 100 nM) were incubated with Supersomes individually expressing the five principal drug metabolizing cytochrome P450s, CYP1A2, 2C9, 2C19, 2D6, and 3A4 and their respective substrates. The catalog number and substrates used for each P450 were as follows: CYP1A2 (P203) and CYP2C19 (P259), 3-Cyano-7-ethoxycoumarin (CEC); CYP2C9 (P258), Dibenzylfluorescein (DBF); CYP2D6 (P217), 3-[2-(N,N-diethyl-N-methylamino)ethyl]-7-methoxy-4-methylcoumarin (AMMC); and CYP3A4 (202), 7-benzyloxy-4-trifluoromethylcoumarin (BFC). Incubations with known inhibitors of each P450 isoform were included as positive controls. Fluorescence per well was measured using a Spectramax Gemini XS (Molecular Devices) and IC₅₀ values of the compounds tested were determined using the 5 point dose response. The sigmoidal dose response curves were generated by non linear regression using a four parameter logistic equation. This assay is further described in Miller et al. (2000) “Fluorometric High-Throughput Screening for Inhibitors of Cytochrome P450” Ann N Y Acad. Sci. 2000;919:26-32 and in Crespi et al. (1997) “Microtiter Plate Assays for Inhibition of Human, Drug-Metabolizing Cytochromes P450” Analytical Biochemistry 248, 188-190.

For comparison, the P450 inhibitory activity of two compounds described in WO 98/35958 (examples number 4 and 50) was also determined.

The P450 inhibitory activity of certain compounds of the invention is set forth in Table 1.

TABLE 1
P450 inhibitory activity of compounds

| | | CYP1A2 | CYP2C9 | CYP2C19 | CYP2D6 | CYP3A4 |
|---|---|---|---|---|---|---|
| | Compound | Inh. | Inh. | Inh. | Inh. | Inh. |
| Structure | described in | IC₅₀ (μM) | IC₅₀ (μM) | IC₅₀ (μM) | IC₅₀ (μM) | IC₅₀ (μM) |
| 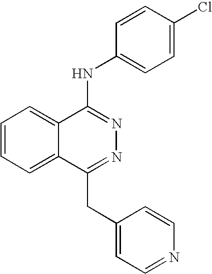 | WO 98/35958 (Example 4) | 1.49 | 0.65 | 0.24 | 0.33 | 0.16 |
| Reference | | | | | | |
| 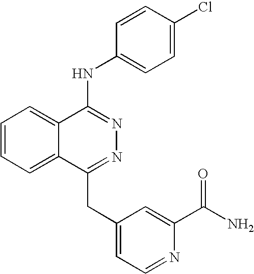 | herein and in WO 01/10859 | >20 | >20 | 15 | >20 | 10 |
| 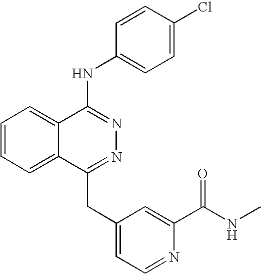 | herein and in WO 01/1085 | >20 | 5.7 | 11 | >20 | 2.2 |
| 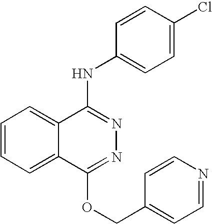 | WO 98/35958 (Example 50) | 0.24 | 0.29 | 0.79 | 0.33 0.07 | |
| Reference | | | | | | |
| 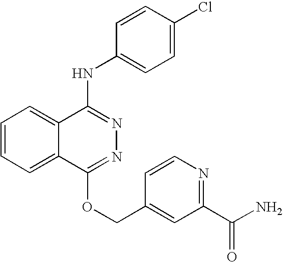 | herein and in WO 01/10859 | 1.5 | 0.95 | 5.4 | 1.3 | 0.42 |
| 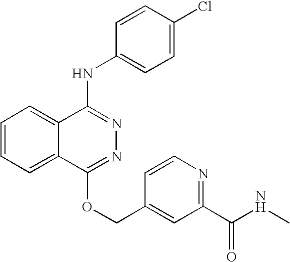 | herein and in WO 01/10859 | >20 | >20 | >20 | >20 | >20 |
| 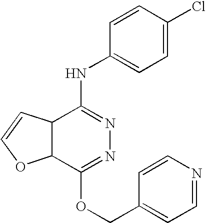 | herein and in WO 01/23375 | 0.66 | 0.68 | 1.7 | 0.78 | 0.39 |
| Reference | | | | | | |
| 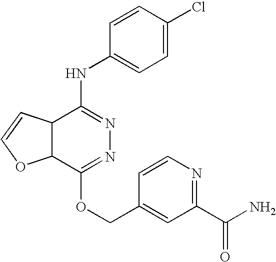 | herein and in WO 01/23375 | 17 | >20 | >20 | >20 | 2.1 |
| 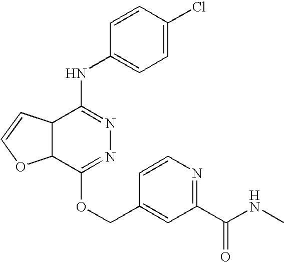 | herein and in WO 01/23375 | >20 | >20 | >20 | >20 | 19 |
| 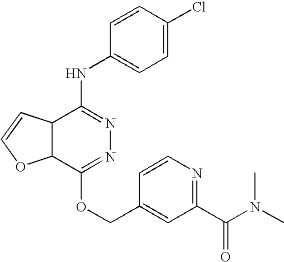 | herein and in WO 01/23375 | 3.5 | >20 | >20 | >20 | 11 |
| 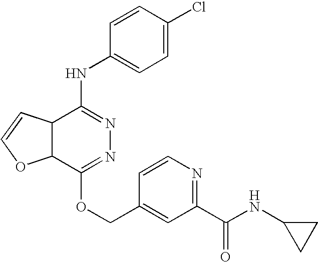 | herein and in WO 01/23375 | >20 | >20 | >20 | >20 | 3.3 |
| 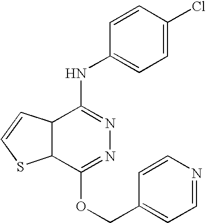 | herein and in WO 01/23375 | 0.50 | 0.46 | 7.5 | 0.40 | 0.12 |
| Reference | | | | | | |
| 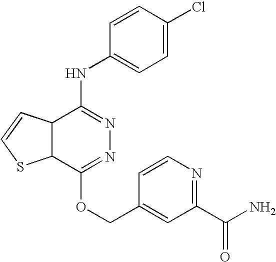 | herein and in WO 01/23375 | 3.3 | 4.7 | >20 | 14 | 1.0 |
| 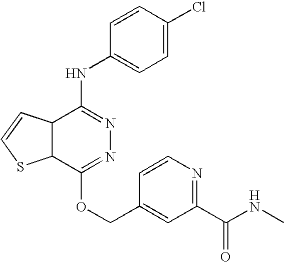 | herein and in WO 01/23375 | >20 | >20 | >20 | >20 | >20 |
| 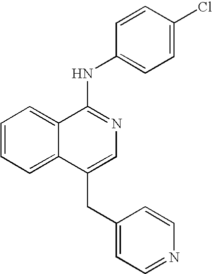 | herein and in WO 01/23375 | 0.17 | 0.11 | 0.07 | 0.21 | 0.14 |
| Reference | | | | | | |
| 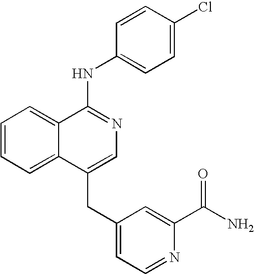 | herein and in WO 01/23375 | 3.3 | 1.9 | 1.2 | 15 | 1.2 |
| 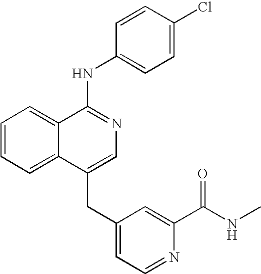 | herein and in WO 01/23375 | 3.8 | 1.6 | 0.85 | 10 | 1.3 |
| 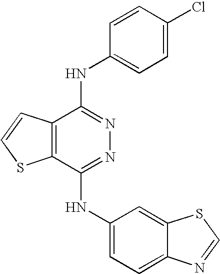 | herein and in WO 01/23375 | >20 | 3.9 | 9.9 | >20 | >20 |
| 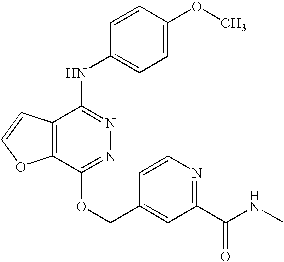 | herein and in WO 01/23375 | 4.2 | >20 | >20 | >20 | 1.8 |
| 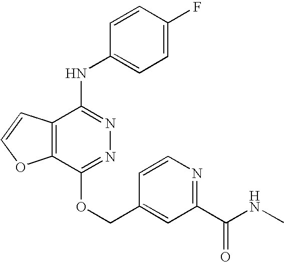 | herein and in WO 01/23375 | >20 | >20 | Assay Interference | >20 | >20 |
| 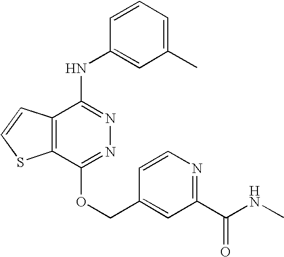 | herein and in WO 01/23375 | >20 | >20 | Assay Interference | >20 | >20 |
| 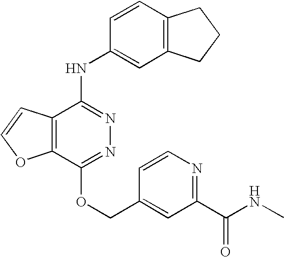 | herein and in WO 01/23375 | 4.5 | 4.0 | >20 | >20 | 0.48 |
| 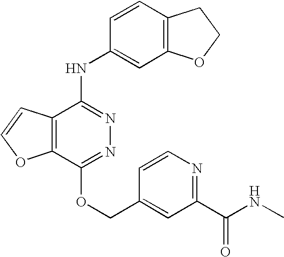 | herein and in WO 01/23375 | 11 | >20 | >20 | >20 | 7.6 |
| Positive Control | | 1.8 | 0.69 | 1.18 | 0.009 | 0.023 |
| | | Furafyline | Sulfaphenazole | Tranylcypromine | Quinidine | Ketocanazole |

Thus, the results show surprisingly, that the compounds described herein that have 2-aminocarbonyl (amide) substituents on the pendant pyridine rings show low levels of P450 inhibition towards many of the P450 enzymes. On the contrary, analogous compounds described herein or in any of the PCT applications (see above) having a pendant pyridine ring but without the amide substituent, generally show much higher P450 inhibition values (low IC₅₀s). This suggests that compounds with amidated pyridine rings as described herein are expected to be especially useful for use in combination therapy with various other chemotherapeutic agents, e.g., for the treatment of cancer.

The results also indicate surprisingly, that compounds described herein that have pendant benzo-fused 5 member ring heterocycles rather than pendant pyridine rings, such as the eigteenth compound in Table 1a, also do not exibit high inhibition of P450 enzymes and are therefore also expected to have a low potential to result in drug drug interactions when given in combination with other chemotherapeutic agents.

Additional compounds that are expected to show low P450 inhibition activity that were exemplified in either WO 012375 or WO 0110859 are shown in Tables 2 through 4, supra.

Equivalents

Those skilled in the art will recognize, or be able to ascertain using no more than routine experimentation, many equivalents of the specific embodiments of the invention described herein. Such equivalents are intended to be encompassed by the following claims.

1. A method for treating a subject having cancer, comprising administering to the subject a therapeutically efficient amount of a first chemotherapeutic agent and a therapeutically efficient amount of a compound, which is different from the first chemotherapeutic compound, having the generalized structural formula 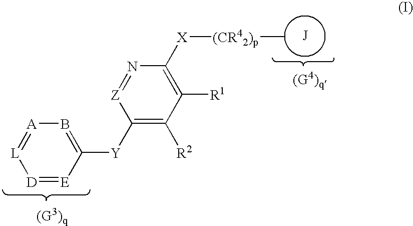
wherein
R¹ and R²:
i) independently represent H or lower alkyl;
ii) together form a bridge of structure 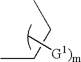
wherein binding is achieved via the terminal carbon atoms;
iii) together form a bridge of structure 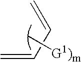
wherein binding is achieved via the terminal carbon atoms;
iv) together form a bridge of structure 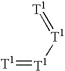
wherein one or two ring members T¹ are N and the others are CH or CG¹, and binding is achieved via the terminal atoms; or
v) together form a bridge containing two T² moieties and one T³ moiety, said bridge, taken together with the ring to which it is attached, forming a bicyclic of structure 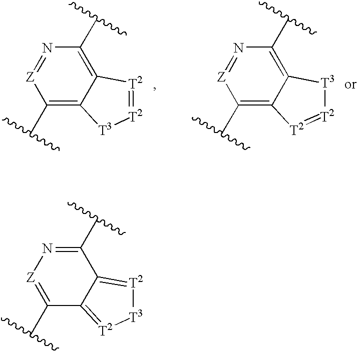
wherein
each T² independently represents N, CH, or CG¹; and
T³ represents S, O, CR⁴G¹, C(R⁴)₂, or NR³;
and wherein
G¹ is a substituent independently selected from the group consisting of
—N(R⁶)₂;
—NR³COR⁶;
halogen;
alkyl;
cycloalkyl;
lower alkenyl;
lower cycloalkenyl;
halogen-substituted alkyl;
amino-substituted alkyl;
N-lower alkylamino-substituted alkyl;
N,N-di-lower alkylamino-substituted alkyl;
N-lower alkanoylamino-substituted alkyl;
hydroxy-substituted alkyl;
cyano-substituted alkyl;
carboxy-substituted alkyl;
lower alkoxycarbonyl-substituted alkyl;
phenyl lower alkoxycarbonyl-substituted alkyl;
halogen-substituted alkylamino;
amino-substituted alkylamino;
N-lower alkylamino-substituted alkylamino;
N,N-di-lower alkylamino-substituted alkylamino;
N-lower alkanoylamino-substituted alkylamino;
hydroxy-substituted alkylamino;
cyano-substituted alkylamino;
carboxy-substituted alkylamino;
lower alkoxycarbonyl-substituted alkylamino;
phenyl-lower alkoxycarbonyl-substituted alkylamino;
—OR⁶;
—SR⁶;
—S(O)R⁶;
—S(O)₂R⁶;
halogenated lower alkoxy;
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
amidino;
guanidino;
sulfo;
—B(OH)₂;
optionally substituted aryl;
optionally substituted heteroaryl;
optionally substituted saturated heterocyclyl;
optionally substituted saturated heterocyclylalkyl;
optionally substituted partially unsaturated heterocyclyl;
optionally substituted partially unsaturated heterocyclylalkyl;
—OCO₂R³;
optionally substituted heteroarylalkyl;
optionally substituted heteroaryloxy;
—S(O)ₚ(optionally substituted heteroaryl);
optionally substituted heteroarylalkyloxy;
—S(O)ₚ(optionally substituted heteroarylalkyl);
—CHO;
—OCON(R⁶)₂;
—NR³CO₂R⁶;
—NR³CON(R⁶)₂
m is 0, 1, 2, 3, or 4;
R³ is H or lower alkyl;
R⁶ is independently selected from the group consisting of
H;
alkyl;
cycloalkyl;
optionally substituted aryl; and
optionally substituted aryl lower alkyl;
lower alkyl-N(R³)₂; and
lower alkyl-OH;
R⁴ is H, halogen, or lower alkyl;
p is 0, 1, or 2;
X is selected from the group consisting of O, S, and NR³;
Y is selected from the group consisting of
lower alkylene;
—CH₂—O—;
—CH₂—S—;
—CH₂—NH—;
—O—;
—S—;
—NH—;
—(CR⁴₂)ₙ—S(O)ₚ-(5-membered heteroaryl)-(CR⁴₂)ₛ—;
—(CR⁴₂)ₙ—C(G²)(R⁴)—(CR⁴₂)ₛ—;
wherein
n and s are each independently 0 or an integer of 1-2; and
G² is selected from the group consisting of —CN, —CO₂R³, —CON(R⁶)₂, and —CH₂N(R⁶)₂;
—O—CH₂—;
—S(O)—;
—S(O)₂—;
—SCH₂—;
—S(O)CH₂—;
—S(O)₂CH₂—;
—CH₂S(O)—; and
—CH₂S(O)₂—
Z is CR⁴ or N;
q is 0, 1, or 2;
G³ is a monovalent or bivalent moiety selected from the group consisting of:
lower alkyl;
—NR³COR⁶;
carboxy-substituted alkyl;
lower alkoxycarbonyl-substituted alkyl;
—OR⁶;
—SR⁶;
—S(O)R⁶;
—S(O)₂R⁶;
—OCOR⁶;
—COR⁶;
—CO₂R³;
—CH₂OR³;
—CON(R⁶)₂;
—S(O)₂N(R⁶)₂;
—NO₂;
—CN;
optionally substituted aryl;
optionally substituted heteroaryl;
optionally substituted saturated heterocyclyl;
optionally substituted partially unsaturated heterocyclyl;
optionally substituted heteroarylalkyl;
optionally substituted heteroaryloxy,
—S(O)ₚ(optionally substituted heteroaryl);
optionally substituted heteroarylalkyloxy,
—S(O)ₚ(optionally substituted heteroarylalkyl);
—OCON(R⁶)₂;
—NR³CO₂R¹;
—NR³CON(R⁶)₂; and
bivalent bridge of structure T=T²-T³
wherein
each T² independently represents N, CH, or CG^{3′}; and
T³ represents S, O, CR⁴G^{3′}, C(R⁴)₂, or NR³; wherein
G^{3′} represents any of the above-defined moieties G³ which are monovalent; and the terminal T² is bound to L, and T³ is bound to D, forming a 5-membered fused ring;
with the proviso that when q is 0 or each G³ is an independent lower alkyl substituent, then R¹ and R² together form a bridge containing two T² moieties and one T³ moiety, said bridge, taken together with the ring to which it is attached, forming a bicyclic of structure 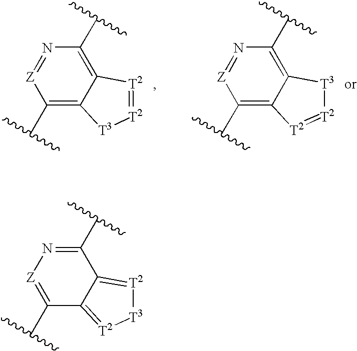
wherein
each T² independently represents N, CH, or CG¹; and T³ represents S, O, CR⁴G¹, C(R⁴)₂, or NR³;
A and D independently represent N or CH;
B and E independently represent N or CH;
L represents N or CH; and
with the provisos that
a) the total number of N atoms in the ring containing A, B, D, E, and L is 0, 1, 2, or 3; and
b) when L represents CH and q is 0 or any G³ is a monovalent substituent, at least one of A and D is an N atom; and
c) when L represents CH and a G³ is a bivalent bridge of structure T²=T²-T³, then A, B, D, and E are also CH;
J is a ring selected from the group consisting of
aryl;
pyridyl; and
cycloalkyl;
q′ represents the number of substituents G⁴ on ring J and is 0, 1, 2, 3, 4, or 5, and
G⁴ is a monovalent or bivalent moiety selected from the group consisting of
—N(R⁶)₂;
—NR³COR⁶;
halogen;
alkyl;
cycloalkyl;
lower alkenyl;
lower cycloalkenyl;
halogen-substituted alkyl;
amino-substituted alkyl;
N-lower alkylamino-substituted alkyl;
N,N-di-lower alkylamino-substituted alkyl;
N-lower alkanoylamino-substituted alkyl;
hydroxy-substituted alkyl;
cyano-substituted alkyl;
carboxy-substituted alkyl;
lower alkoxycarbonyl-substituted alkyl;
phenyl lower alkoxycarbonyl-substituted alkyl;
halogen-substituted alkylamino;
amino-substituted alkylamino;
N-lower alkylamino-substituted alkylamino;
N,N-di-lower alkylamino-substituted alkylamino;
N-lower alkanoylamino-substituted alkylamino;
hydroxy-substituted alkylamino;
cyano-substituted alkylamino;
carboxy-substituted alkylamino;
lower alkoxycarbonyl-substituted alkylamino;
phenyl-lower alkoxycarbonyl-substituted alkylamino;
—OR⁶;
—SR⁶;
—S(O)R⁶;
—S(O)₂R⁶;
halogenated lower alkoxy;
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
amidino;
guanidino;
sulfo;
—B(OH)₂;
optionally substituted aryl;
optionally substituted heteroaryl;
optionally substituted saturated heterocyclyl;
optionally substituted partially unsaturated heterocyclyl;
—OCO₂R³;
optionally substituted heteroarylalkyl;
optionally substituted heteroaryloxy;
—S(O)ₚ(optionally substituted heteroaryl);
optionally substituted heteroarylalkyloxy;
—S(O)ₚ(optionally substituted heteroarylalkyl);
—CHO;
—OCON(R⁶)₂;
—NR³CO₂R⁶;
—NR³CON(R⁶)₂; and
fused ring-forming bivalent bridges attached to and connecting adjacent positions of ring J, said bridges having the structures: 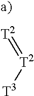
wherein
each T² independently represents N, CH, or CG^{4′};
T³ represents S, O, CR⁴G^{4′}, C(R⁴)₂, or NR³; wherein
G4′ represents any of the above-defined moieties G⁴ which are monovalent; and binding to ring J is achieved via terminal atoms T² and T³; 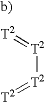
wherein
each T² independently represents N, CH, or CG^{4′}; wherein
G4′ represents any of the above-defined moieties G⁴ which are monovalent; and with the proviso that a maximum of two bridge atoms T² may be N; and
binding to ring J is achieved via terminal atoms T²; and 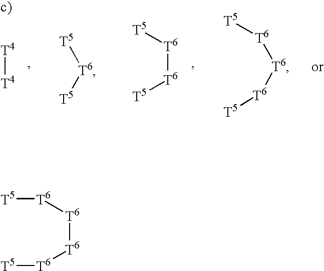
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and binding to ring J is achieved via terminal atoms T⁴ or T⁵;
with the provisos that:
i) when one T⁴ is O, S, or NR³, the other T⁴ is CR⁴G^{4′} or C(R⁴)₂;
ii) a bridge comprising T⁵ and T⁶ atoms may contain a maximum of two heteroatoms O, S, or N; and
iii) in a bridge comprising T⁵ and T⁶ atoms, when one T⁵ group and one T⁶ group are O atoms, or two T⁶ groups are O atoms, said O atoms are separated by at least one carbon atom;
when G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members;
and with the further provisos that:
in G¹, G², G³, and G⁴, when two groups R³ or R⁶ are each alkyl and located on the same N atom they may be linked by a bond, an O, an S, or NR³ to form a N-containing heterocycle of 5-7 ring atoms;
when an aryl, heteroaryl, or heterocyclyl ring is optionally substituted, that ring may bear up to 5 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂R³, —CHO, —CH₂OR³, —OCO₂R³, —CON(R⁶)₂, —OCON(R⁶)₂, —NR³CON(R⁶)₂, nitro, amidino, guanidino, mercapto, sulfo, and cyano; and
when any alkyl group is attached to O, S, or N, and bears a hydroxyl substituent, then said hydroxyl substituent is separated by at least two carbon atoms from the O, S, or N to which the alkyl group is attached.

2. The method of claim 1, wherein R¹ and R²
together form a bridge containing two T² moieties and one T³ moiety, said bridge, taken together with the ring to which it is attached, forming a bicyclic of structure 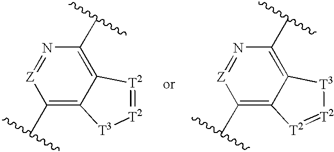
wherein
each T² independently represents N, CH, or CG¹; and
T³ represents S, O, CH₂, or NR³;
with the proviso that when T³ is O or S, at least one T² is CH or CG¹.

3. The method of claim 1, wherein
R¹ and R²
i) together form a bridge of structure 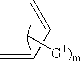
wherein binding is achieved via the terminal carbon atoms; or
ii) together form a bridge of structure 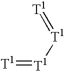
wherein one or two ring members T¹ are N and the others are CH or CG¹, and binding is achieved via the terminal atoms.

4. The method of claim 1, wherein
q is 1 or 2; A, B, D, and E are CH; L is N; one G³ is found on ring position D; and that G³ is —CON(R⁶)₂.

5. The method of claim 1, wherein
q is 1 or 2; A, B, D, E, and L are CH; and one of the G³ forms
a bivalent bridge of structure T²=T²-T³
wherein
each T² independently represents N, CH, or CG^{3′}; and
T³ represents S, O, CR⁴G^{3′}, C(R⁴)₂, or NR³; wherein
G^{3′} represents any of the above-defined moieties G³ which are monovalent; and the terminal T² is bound to L, and T³ is bound to D, forming a 5-membered fused ring.

6. The method of claim 4, wherein
p is 0; J is phenyl; Z is CH or N; Y is selected from a group consisting of
lower alkylene;
—CH₂—O—;
—CH₂—S—;
—CH₂—NH—;
—O—;
—S—;
—NH—;
G¹ is selected from a group consisting of
—N(R⁶)₂;
alkyl;
amino-substituted alkyl;
N-lower alkylamino-substituted alkyl;
N,N-di-lower alkylamino-substituted alkyl;
hydroxy-substituted alkyl;
carboxy-substituted alkyl;
amino-substituted alkylamino;
N-lower alkylamino-substituted alkylamino;
N,N-di-lower alkylamino-substituted alkylamino;
hydroxy-substituted alkylamino;
carboxy-substituted alkylamino;
—OR⁶;
—S(O)R⁶;
—S(O)₂R⁶;
—OCOR⁶;
—COR⁶;
—CO₂R⁶;
—CON(R⁶)₂;
—CH₂OR³;
—NO₂;
—CN;
amidino;
guanidino;
sulfo;
optionally substituted heteroaryl;
optionally substituted saturated heterocyclyl;
optionally substituted saturated heterocyclylalkyl;
optionally substituted partially unsaturated heterocyclyl;
optionally substituted partially unsaturated heterocyclylalkyl;
optionally substituted heteroarylalkyl;
optionally substituted heteroaryloxy,
—S(O)ₚ(optionally substituted heteroaryl);
optionally substituted heteroarylalkyloxy;
—S(O)ₚ(optionally substituted heteroarylalkyl);
—OCON(R⁶)₂;
—NR³CO₂R⁶; and
—NR³CON(R⁶)₂;
any additional G³ is selected from a group consisting of
halogen;
lower alkyl;
hydroxyl; and
lower alkoxy;
G⁴ is selected from a group consisting of
halogen;
alkyl;
cycloalkyl;
lower alkenyl;
lower cycloalkenyl;
halogen-substituted alkyl;
hydroxy-substituted alkyl;
cyano-substituted alkyl;
lower alkoxycarbonyl-substituted alkyl;
phenyl lower alkoxycarbonyl-substituted alkyl;
—OR⁶;
—SR⁶;
—S(O)R⁶;
—S(O)₂R⁶;
halogenated lower alkoxy;
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
optionally substituted aryl;
optionally substituted heteroaryl;
optionally substituted saturated heterocyclyl;
optionally substituted partially unsaturated heterocyclyl;
optionally substituted heteroarylalkyl;
optionally substituted heteroaryloxy;
—S(O)ₚ(optionally substituted heteroaryl);
optionally substituted heteroarylalkyloxy;
—S(O)ₚ(optionally substituted heteroarylalkyl);
—OCON(R⁶)₂;
—NR³CO₂R⁶;
—NR³CON(R⁶)₂; and
fused ring-forming bivalent bridges attached to and connecting adjacent positions of ring J, said bridges having the structures: 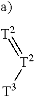
wherein
each T² independently represents N, CH, or CG^{4′};
T³ represents S, O, CR⁴G^{4′}, C(R⁴)₂, or NR³; wherein
G4′ represents any of the above-defined moieties G⁴ which are monovalent; and
binding to ring J is achieved via terminal atoms T² and T³; 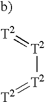
wherein
each T² independently represents N, CH, or CG^{4′}; wherein
G4′ represents any of the above-defined moieties G⁴ which are monovalent; and
with the proviso that a maximum of two bridge atoms T² may be N; and
binding to ring J is achieved via terminal atoms T²; and 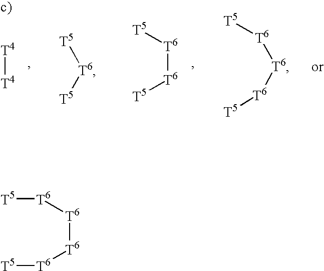
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and
binding to ring J is achieved via terminal atoms T⁴ or T⁵;
with the provisos that:
i) when one T⁴ is O, S, or NR³, the other T⁴ is CR⁴G^{4′} or C(R⁴)₂;
ii) a bridge comprising T⁵ and T⁶ atoms may contain a maximum of two heteroatoms O, S, or N; and
iii) in a bridge comprising T⁵ and T⁶ atoms, when one T⁵ group and one T⁶ group are O atoms, or two T⁶ groups are O atoms, said O atoms are separated by at least one carbon atom;
when G⁴ is an alkyl group located on ring J adjacent to the linkage —(CR⁴₂)ₚ—, and X is NR³ wherein R³ is an all substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members;
and R¹ and R²:
i) together form a bridge of structure 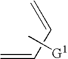
wherein binding is achieved via the terminal carbon atoms;
ii) together form a bridge of structure 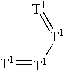
wherein one ring member T¹ is N and the others are CH or CG¹, and binding is achieved via the terminal atoms; or
iii) together form a bridge containing two T² moieties and one T³ moiety, said bridge, taken together with the ring to which it is attached, forming a bicyclic of structure 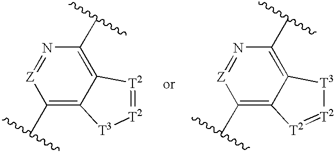
wherein
each T² independently represents N, CH, or CG¹; and
T³ represents S, O, or NR³.

7. The method of claim 5, wherein
p is 0; J is phenyl; Z is CH or N; Y is selected from a group consisting of
lower alkylene;
—CH₂—O—;
—CH₂—S—;
—CH₂—NH—;
—O—;
—S—; and
—NH—;
G¹ is selected from a group consisting of
—N(R⁶)₂;
alkyl;
amino-substituted alkyl;
N-lower alkylamino-substituted alkyl;
N,N-di-lower alkylamino-substituted alkyl;
hydroxy-substituted alkyl;
carboxy-substituted alkyl;
amino-substituted alkylamino;
N-lower alkylamino-substituted alkylamino;
N,N-di-lower alkylamino-substituted alkylamino;
hydroxy-substituted alkylamino;
carboxy-substituted alkylamino;
—OR⁶;
—S(O)R⁶;
—S(O)₂R⁶;
—OCOR⁶;
—COR⁶;
—CO₂R⁶;
—CON(R⁶)₂;
—CH₂OR³;
—NO₂;
—CN;
amidino;
guanidino;
sulfo;
optionally substituted heteroaryl;
optionally substituted saturated heterocyclyl;
optionally substituted saturated heterocyclylalkyl;
optionally substituted partially unsaturated heterocyclyl;
optionally substituted partially unsaturated heterocyclylalkyl;
optionally substituted heteroarylalkyl;
optionally substituted heteroaryloxy;
—S(O)ₚ(optionally substituted heteroaryl);
optionally substituted heteroarylalkyloxy;
—S(O)ₚ(optionally substituted heteroarylalkyl);
—OCON(R⁶)₂;
—NR³CO₂R⁶; and
—NR³CON(R⁶)₂;
any additional G³ is selected from a group consisting of
halogen;
lower alkyl;
hydroxyl; and
lower alkoxy;
G⁴ is selected from a group consisting of
halogen;
alkyl;
cycloalkyl;
lower alkenyl;
lower cycloalkenyl;
halogen-substituted alkyl;
hydroxy-substituted alkyl;
cyano-substituted alkyl;
lower alkoxycarbonyl-substituted alkyl;
phenyl lower alkoxycarbonyl-substituted alkyl;
—OR⁶;
—SR⁶;
—S(O)R⁶;
—S(O)₂R⁶;
halogenated lower alkoxy;
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
optionally substituted aryl;
optionally substituted heteroaryl;
optionally substituted saturated heterocyclyl;
optionally substituted partially unsaturated heterocyclyl;
optionally substituted heteroarylalkyl;
optionally substituted heteroaryloxy;
—S(O)ₚ(optionally substituted heteroaryl);
optionally substituted heteroarylalkyloxy;
—S(O)ₚ(optionally substituted heteroarylalkyl);
—OCON(R⁶)₂;
—NR³CO₂R⁶;
—NR³CON(R⁶)₂; and
fused ring-forming bivalent bridges attached to and connecting adjacent positions of ring J, said bridges having the structures: 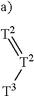
wherein
each T² independently represents N, CH, or CG^{4′};
T³ represents S, O, CR⁴G^{4′}, C(R⁴)₂, or NR³; wherein
G4′ represents any of the above-defined moieties G⁴ which are monovalent; and
binding to ring J is achieved via terminal atoms T² and T³; 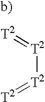
wherein
each T² independently represents N, CH, or CG^{4′}; wherein
G4′ represents any of the above-defined moieties G⁴ which are monovalent; and
with the proviso that a maximum of two bridge atoms T² may be N; and
binding to ring J is achieved via terminal atoms T²; and 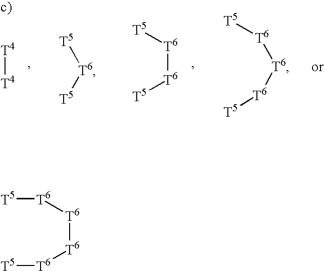
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and
binding to ring J is achieved via terminal atoms T⁴ or T⁵;
with the provisos that:
i) when one T⁴ is O, S, or NR³, the other T⁴ is CR⁴G^{4′} or C(R⁴)₂;
ii) a bridge comprising T⁵ and T⁶ atoms may contain a maximum of two heteroatoms O, S, or N; and
iii) in a bridge comprising T⁵ and T⁶ atoms, when one T⁵ group and one T⁶ group are O atoms, or two T⁶ groups are O atoms, said O atoms are separated by at least one carbon atom;
when G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)ₚ′— wherein p′ is 2, 3, or 4, with the proviso that the sum of p and p′ is 2, 3, or 4, resulting in formation of a nitrogen-containing ring of 5, 6, or 7 members;
and R¹ and R²:
i) together form a bridge of structure 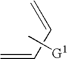
wherein binding is achieved via the terminal carbon atoms;
ii) together form a bridge of structure 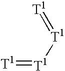
wherein one ring member T¹ is N and the others are CH or CG¹, and binding is achieved via the terminal atoms; or
iii) together form a bridge containing two T² moieties and one T³ moiety, said bridge, taken together with the ring to which it is attached, forming a bicyclic of structure 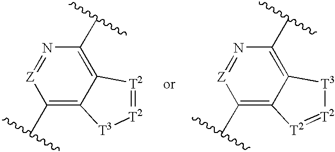
wherein
each T² independently represents N, CH, or CG¹; and
T³ represents S, O, or NR³.

8. A method for treating a subject having cancer, comprising administering to the subject a therapeutically efficient amount of a first chemotherapeutic agent and a therapeutically efficient amount of a compound, which is different from the first chemotherapeutic compound, having a generalized structural formula selected from the group consisting of 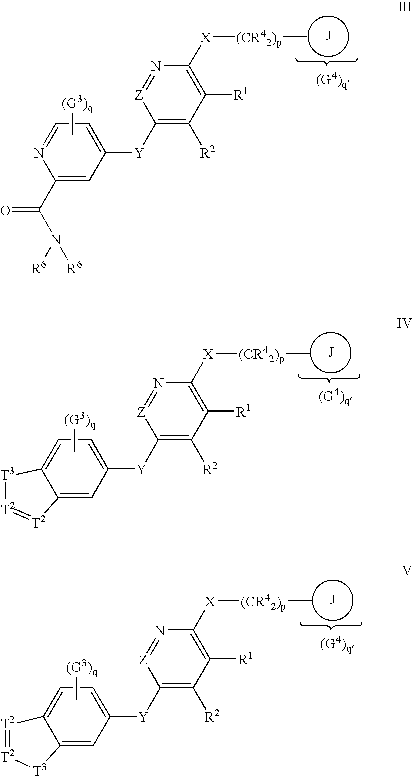
wherein
R¹ and R²:
i) independently represent H or lower alkyl;
ii) together form a bridge of structure 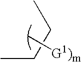
wherein binding is achieved via the terminal carbon atoms;
iii) together form a bridge of structure 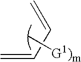
wherein binding is achieved via the terminal carbon atoms;
iv) together form a bridge of structure 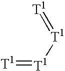
wherein one or two ring members T¹ are N and the others are CH or CG¹, and binding is achieved via the terminal atoms; or
v) together form a bridge containing two T² moieties and one T³ moiety, said bridge, taken together with the ring to which it is attached, forming a bicyclic of structure 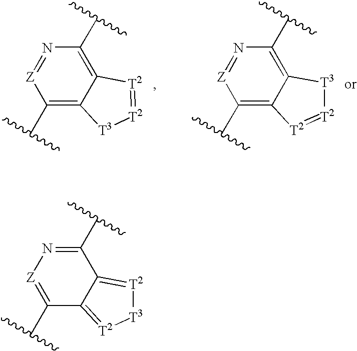
wherein
each T² independently represents N, CH, or CG¹; and
T³ represents S, O, CR⁴G¹, C(R⁴)₂, or NR³;
and wherein
G¹ is a substituent independently selected from the group consisting of
—N(R⁶)₂;
—NR³COR⁶;
halogen;
alkyl;
cycloalkyl;
lower alkenyl;
lower cycloalkenyl;
halogen-substituted alkyl;
amino-substituted alkyl;
N-lower alkylamino-substituted alkyl;
N,N-di-lower alkylamino-substituted alkyl;
N-lower alkanoylamino-substituted alkyl;
hydroxy-substituted alkyl;
cyano-substituted alkyl;
carboxy-substituted alkyl;
lower alkoxycarbonyl-substituted alkyl;
phenyl lower alkoxycarbonyl-substituted alkyl;
halogen-substituted alkylamino;
amino-substituted alkylamino;
N-lower alkylamino-substituted alkylamino;
N,N-di-lower alkylamino-substituted alkylamino;
N-lower alkanoylamino-substituted alkylamino;
hydroxy-substituted alkylamino;
cyano-substituted alkylamino;
carboxy-substituted alkylamino;
lower alkoxycarbonyl-substituted alkylamino;
phenyl-lower alkoxycarbonyl-substituted alkylamino;
—OR⁶;
—SR⁶;
—S(O)R⁶;
—S(O)₂R⁶;
halogenated lower alkoxy,
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
amidino;
guanidino;
sulfo;
—B(OH)₂;
optionally substituted aryl;
optionally substituted heteroaryl;
optionally substituted saturated heterocyclyl;
optionally substituted saturated heterocyclylalkyl;
optionally substituted partially unsaturated heterocyclyl;
optionally substituted partially unsaturated heterocyclylalkyl;
—OCO₂R³;
optionally substituted heteroarylalkyl;
optionally substituted heteroaryloxy;
—S(O)ₚ(optionally substituted heteroaryl);
optionally substituted heteroarylalkyloxy;
—S(O)ₚ(optionally substituted heteroarylalkyl);
—CHO;
—OCON(R⁶)₂;
—NR³CO₂⁶; and
—NR³CON(R⁶)₂
R³ is H or lower alkyl;
R⁶ is independently selected from the group consisting of
H;
alkyl;
cycloalkyl;
optionally substituted aryl; and
optionally substituted aryl lower alkyl;
lower alkyl-N(R³)₂; and
lower alkyl-OH;
R⁴ is H, halogen, or lower alkyl;
p is 0, 1, or 2;
X is selected from the group consisting of O, S, and NR³;
Y is selected from the group consisting of
lower alkylene;
—CH₂—O—;
—CH₂—S—;
—CH₂—NH—;
—O—;
—S—;
—NH—;
—(CR⁴₂)ₙ—S(O)ₚ-(5-membered heteroaryl)-(CR⁴₂)ₛ—;
—(CR⁴₂)ₙ—C(G²)(R⁴)—(CR⁴₂)ₛ—;
wherein
n and s are each independently 0 or an integer of 1-2; and (2 is selected from the group consisting of —CN, —CO₂R³, —CON(R⁶)₂, and —CH₂N(R⁶)₂;
—O—CH₂—;
—S(O)—;
—S(O)₂—;
—SCH₂—;
—S(O)CH₂—;
—S(O)₂CH₂—;
—CH₂S(O)—; and
—CH₂S(O)₂—
Z is CH, CG³, or N;
q is 0 or 1;
G³ is a monovalent moiety selected from the group consisting of:
lower alkyl;
—NR³COR⁶;
carboxy-substituted alkyl;
lower alkoxycarbonyl-substituted allyl;
—OR⁶;
—SR⁶;
—S(O)R⁶;
—S(O)₂R⁶;
—OCOR⁶;
—COR⁶;
—CO₂R⁶;
—CH₂OR³;
—CON(R⁶)₂;
—S(O)₂N(R⁶)₂;
—NO₂;
—CN;
optionally substituted aryl;
optionally substituted heteroaryl;
optionally substituted saturated heterocyclyl;
optionally substituted partially unsaturated heterocyclyl;
optionally substituted heteroarylalkyl;
optionally substituted heteroaryloxy,
—S(O)ₚ(optionally substituted heteroaryl);
optionally substituted heteroarylalkyloxy;
—S(O)ₚ(optionally substituted heteroarylalkyl);
—OCON(R⁶)₂;
—NR³CO₂R⁶; and
—NR³CON(R⁶)₂;
J is a ring selected from the group consisting of
aryl;
pyridyl; and
cycloalkyl;
q′ represents the number of substituents G⁴ on ring J and is 0, 1, 2, 3, 4, or 5, and
G⁴ is a monovalent or bivalent moiety selected from the group consisting of
—N(R⁶)₂;
—NR³COR⁶;
halogen;
alkyl;
cycloalkyl;
lower alkenyl;
lower cycloalkenyl;
halogen-substituted alkyl;
amino-substituted alkyl;
N-lower alkylamino-substituted alkyl;
N,N-di-lower alkylamino-substituted alkyl;
N-lower alkanoylamino-substituted alkyl;
hydroxy-substituted alkyl;
cyano-substituted alkyl;
carboxy-substituted alkyl;
lower alkoxycarbonyl-substituted alkyl;
phenyl lower alkoxycarbonyl-substituted alkyl;
halogen-substituted alkylamino;
amino-substituted alkylamino;
N-lower alkylamino-substituted alkylamino;
N,N-di-lower alkylamino-substituted alkylamino;
N-lower alkanoylamino-substituted alkylamino;
hydroxy-substituted alkylamino;
cyano-substituted alkylamino;
carboxy-substituted alkylamino;
lower alkoxycarbonyl-substituted alkylamino;
phenyl-lower alkoxycarbonyl-substituted alkylamino;
—OR⁶;
—SR⁶;
—S(O)R⁶;
—S(O)₂R⁶;
halogenated lower alkoxy;
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
amidino;
guanidino;
sulfo;
—B(OH)₂;
optionally substituted aryl;
optionally substituted heteroaryl;
optionally substituted saturated heterocyclyl;
optionally substituted partially unsaturated heterocyclyl;
—OCO₂R³;
optionally substituted heteroarylalkyl;
optionally substituted heteroaryloxy,
—S(O)ₚ(optionally substituted heteroaryl);
optionally substituted heteroarylalkyloxy;
—S(O)ₚ(optionally substituted heteroarylalkyl);
—CHO;
—OCON(R⁶)₂;
—NR³CO₂R⁶;
—NR³CON(R⁶)₂; and
fused ring-forming bivalent bridges attached to and connecting adjacent positions of ring J, said bridges having the structures: 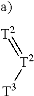
wherein
each T² independently represents N, CH, or CG^{4′};
T³ represents S, O, CR⁴G^{4′}, C(R⁴)₂, or NR³; wherein
G⁴′ represents any of the above-defined moieties G⁴ which are monovalent; and
binding to ring J is achieved via terminal atoms T² and T³; 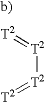
wherein
each T² independently represents N, CH, or CG^{4′}; wherein
G4′ represents any of the above-defined moieties G⁴ which are monovalent; and
with the proviso that a maximum of two bridge atoms T² may be N; and
binding to ring J is achieved via terminal atoms T²; and 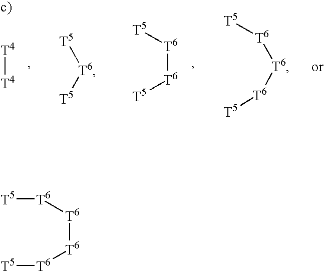
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and
binding to ring J is achieved via terminal atoms T⁴ or T⁵;
with the provisos that:
i) when one T⁴ is O, S, or NR³, the other T⁴ is CR⁴G^{4′} or C(R⁴)₂;
ii) a bridge comprising T⁵ and T⁶ atoms may contain a maximum of two heteroatoms O, S, or N; and
iii) in a bridge comprising T⁵ and T⁶ atoms, when one T⁵ group and one T⁶ group are O atoms, or two T⁶ groups are O atoms, said O atoms are separated by at least one carbon atom;
when G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members;
and with the further provisos that:
in G¹, G², and G⁴, when two groups R³ or R⁶ are each alkyl and located on the same N atom they may be linked by a bond, an O, an S, or NR³ to form a N-containing heterocycle of 5-7 ring atoms;
when an aryl, heteroaryl, or heterocyclyl ring is optionally substituted, that ring may bear up to 5 substituents which are independently selected from the group consisting of amino, mono-loweralkyl-substituted amino, di-loweralkyl-substituted amino, lower alkanoylamino, halogeno, lower alkyl, halogenated lower alkyl, hydroxy, lower alkoxy, lower alkylthio, halogenated lower alkoxy, halogenated lower alkylthio, lower alkanoyloxy, —CO₂R³, —CHO, —CH₂OR³, —OCO₂R³, —CON(R⁶)₂, —OCON(R⁶₂, —NR³CON(R⁶)₂, nitro, amidino, guanidino, mercapto, sulfo, and cyano; and
when any alkyl group is attached to O, S, or N, and bears a hydroxyl substituent, then said hydroxyl substituent is separated by at least two carbon atoms from the O, S, or N to which the alkyl group is attached.

9. The method of claim 8, wherein p is 0; J is phenyl or cycloalkyl; and R¹ and R²
i) together form a bridge containing two T² moieties and one T³ moiety, said bridge, taken together with the ring to which it is attached, forming a bicyclic of structure 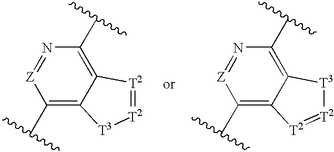
wherein
each T² independently represents N, CH, or CG¹; and
T³ represents S, O, CH₂, or NR³;
with the proviso that when T³ is O or S, at least one s is CH or CG¹; or
ii) together form a bridge of structure 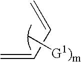
wherein binding is achieved via the terminal carbon atoms; or
iii) together form a bridge of structure 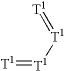
wherein one or two ring members T¹ are N and the others are CH or CG¹, and binding is achieved via the terminal atoms.

10. The method of claim 9, wherein Y is selected from a group consisting of
lower alkylene;
—CH₂—O—;
—CH₂—S—;
—CH₂—NH—;
—O—;
—S—; and
—NH—;
G¹ is selected from a group consisting of
—N(R⁶)₂;
alkyl;
amino-substituted alkyl;
N-lower alkylamino-substituted alkyl;
N,N-di-lower alkylamino-substituted alkyl;
hydroxy-substituted alkyl;
carboxy-substituted alkyl;
amino-substituted alkylamino;
N-lower alkylamino-substituted alkylamino;
N,N-di-lower alkylamino-substituted alkylamino;
hydroxy-substituted alkylamino;
carboxy-substituted alkylamino;
—OR⁶;
—S(O)R⁶;
—S(O)₂R⁶;
—OCOR⁶;
—COR⁶;
—CO₂R⁶;
—CON(R⁶)₂;
—CH₂OR³;
—NO₂;
—CN;
amidino;
guanidino;
sulfo;
optionally substituted heteroaryl;
optionally substituted saturated heterocyclyl;
optionally substituted saturated heterocyclylalkyl;
optionally substituted partially unsaturated heterocyclyl;
optionally substituted partially unsaturated heterocyclylalkyl;
optionally substituted heteroarylalkyl;
optionally substituted heteroaryloxy;
—S(O)ₚ(optionally substituted heteroaryl);
optionally substituted heteroarylalkyloxy,
—S(O)ₚ(optionally substituted heteroarylalkyl);
—OCON(R⁶)₂;
—NR³CO₂R⁶; and
—NR³CON(R⁶)₂;
G³ is selected from a group consisting of
hydroxyl;
lower alkyl; and
lower alkoxy;
G⁴ is selected from a group consisting of
halogen;
alkyl;
cycloalkyl;
lower alkenyl;
lower cycloalkenyl;
halogen-substituted alkyl;
hydroxy-substituted alkyl;
cyano-substituted alkyl;
lower alkoxycarbonyl-substituted alkyl;
phenyl lower alkoxycarbonyl-substituted alkyl;
—OR⁶;
—SR⁶;
—S(O)R⁶;
—S(O)₂R⁶;
halogenated lower alkoxy,
halogenated lower alkylthio;
halogenated lower alkylsulfonyl;
—OCOR⁶;
—COR⁶;
—CO₂R⁶;
—CON(R⁶)₂;
—CH₂OR³;
—NO₂;
—CN;
optionally substituted aryl;
optionally substituted heteroaryl;
optionally substituted saturated heterocyclyl;
optionally substituted partially unsaturated heterocyclyl;
optionally substituted heteroarylalkyl;
optionally substituted heteroaryloxy;
—S(O)ₚ(optionally substituted heteroaryl);
optionally substituted heteroarylalkyloxy,
—S(O)ₚ(optionally substituted heteroarylalkyl);
—OCON(R⁶)₂;
—NR³CO₂R⁶;
—NR³CON(R⁶)₂; and
fused ring-forming bivalent bridges attached to and connecting adjacent positions of ring J, said bridges having the structures: 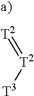
wherein
each T² independently represents N, CH, or CG^{4′};
T³ represents S, O, CR⁴G^{4′}, C(R⁴)₂, or NR³; wherein
G4′ represents any of the above-defined moieties G⁴ which are monovalent; and
binding to ring J is achieved via terminal atoms T² and T³; 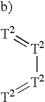
wherein
each T² independently represents N, CH, or CG^{4′}; wherein
G4′ represents any of the above-defined moieties G⁴ which are monovalent; and
with the proviso that a maximum of two bridge atoms T² may be N; and
binding to ring J is achieved via terminal atoms T²; and 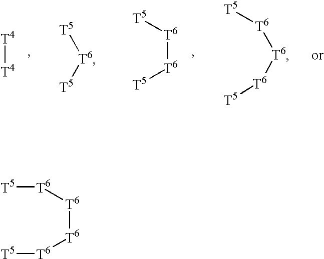
wherein
each T⁴, T⁵, and T⁶ independently represents O, S, CR⁴G^{4′}, C(R⁴)₂, or NR³; wherein
G4′ represents any of the above-defined moieties G⁴ which are monovalent; and
binding to ring J is achieved via terminal atoms T⁴ or T⁵;
with the provisos that:
i) when one T⁴ is O, S, or NR³, the other T⁴ is CR⁴G^{4′} or C(R⁴)₂;
ii) a bridge comprising T⁵ and T⁶ atoms may contain a maximum of two heteroatoms O, S, or N; and
iii) in a bridge comprising T⁵ and T⁶ atoms, when one T⁵ group and one T⁶ group are O atoms, or two T⁶ groups are O atoms, said O atoms are separated by at least one carbon atom;
when G⁴ is an alkyl group located on ring J adjacent to the linkage —(CR⁴₂)ₚ—, and X is NR³ wherein R³ is an alkyl substituent, then G⁴ and the alkyl substituent R³ on X may be joined to form a bridge of structure —(CH₂)_{p′}— wherein p′ is 2, 3, or 4, with the proviso that the sum of p and p′ is 2, 3, or 4, resulting in formation of a nitrogen-containing ring of 5, 6, or 7 members.

11. The method of claim 10, wherein the compound is selected from the group of compounds consisting of:
4-({4-[(4-chlorophenyl)amino]-1-phthalazinyl}methyl)-N-methyl-2-pyridinecarboxamide;
4-({4-[(4-chlorophenyl)amino]-1-phthalazinyl}methyl)-2-pyridinecarboxamide;
4-[({4-[(4-chlorophenyl)amino]-1-phthalazinyl}oxy)methyl]-N-methyl-2-pyridinecarboxamide;
4-[({4-[(4-chlorophenyl)amino]-1-phthalazinyl}oxy)methyl]-2-pyridinecarboxamide;
4-({4-[(3-bromophenyl)amino]-1-phthalazinyl}methyl)-N-methyl-2-pyridinecarboxamide;
4-({4-[(3-bromophenyl)amino]-1-phthalazinyl}methyl)-2-pyridinecarboxamide;
4-({4-[(4-chlorophenyl)amino]-1-phthalazinyl}methyl)-N-methyl-2-pyridinecarboxamide dihydrochloride;
4-({4-[(4-chlorophenyl)amino]-1-phthalazinyl}methyl)-N-methyl-2-pyridinecarboxamide dimethanesulfonate;
4-({4-[(4-chlorophenyl)amino]-1-phthalazinyl}methyl)-2-pyridinecarboxamide dihydrochloride;
4-({4-[(4-chlorophenyl)amino]-1-phthalazinyl}methyl)-2-pyridinecarboxamide dimethanesulfonate;
4-[({4-[(4-chlorophenyl)amino]-1-phthalazinyl}oxy)methyl]-2-pyridinecarboxamidedihydrochloride;
4-[({4-[(4-chlorophenyl)amino]-1-phthalazinyl}oxy)methyl]-2-pyridinecarboxamidedimethanesulfonate;
4-[({4-[(4-chlorophenyl)amino]thieno[2,3-d]pyridazin-7-yl}oxy)methyl]-2-pyridinecarboxamide;
4-[({4-[(4-chlorophenyl)amino]thieno[2,3-d]pyridazin-7-yl}oxy)methyl]-N-methyl-2-pyridinecarboxamide;
4-[({4-[(4-chlorophenyl)amino]furo[2,3-d]pyridazin-7-yl}oxy)methyl]-N-methyl-2-pyridinecarboxamide;
4-[({4-[(4-chlorophenyl)amino]furo[2,3-d]pyridazin-7-yl}oxy)methyl]-2-pyridinecarboxamide;
N-(1,3-benzothiazol-6-yl)-N-{4-[(4-chlorophenyl)amino]thieno[2,3-d]pyridazin-7-yl}amine;
N-(1,3-benzothiazol-6-yl)-N-[4-(2,3-dihydro-1H-inden-5-ylamino)thieno[2,3-d]pyridazin-7-yl]amine;
4-[({4-[(4-methoxyphenyl)amino]furo[2,3-d]pyridazin-7-yl}oxy)methyl]-N-methyl-2-pyridinecarboxamide;
4-[({4-[(4-methoxyphenyl)amino]furo[2,3-a]pyridazin-7-yl}oxy)methyl]-2-pyridinecarboxamide;
N⁷-(1,3-benzothiazol-6-yl)-N′-(4-chlorophenyl)thieno[2,3-d]pyridazine-4,7-diamine;
N-(1,3-benzothiazol-6-yl)-N-[4-(2,3-dihydro-1H-inden-5-ylamino)thieno[2,3-d]pyridazin-7-yl]amine;
N-(1H-indazol-5-yl)-N-[4-(1H-indazol-5-ylamino)thieno[2,3-d]pyridazin-7-yl]amine;
N-(1,3-benzothiazol-6-yl)-N-[4-(1,3-benzothiazol-6-ylamino)furo[2,3-d]pyridazin-7-yl]amine;
4-[({4-[(4-methoxyphenyl)amino]furo[2,3-d]pyridazin-7-yl}oxy)methyl]-N-methyl-2-pyridinecarboxamide;
4-[({4-[(3-chlorophenyl)amino]furo[2,3-d]pyridazin-7-yl}oxy)methyl]-N-methyl-2-pyridinecarboxamide;
4-[({4-[(3-chloro-4-fluorophenyl)amino]furo[2,3-d]pyridazin-7-yl}oxy)methyl]-N-methyl-2-pyridinecarboxamide;
4-[({4-[(4-fluorophenyl)amino]furo[2,3-d]pyridazin-7-yl}oxy)methyl]-N-methyl-2-pyridinecarboxamide;
4-[({4-[(4-bromophenyl)amino]furo[2,3-d]pyridazin-7-yl}oxy)methyl]-N-methyl-2-pyridinecarboxamide;
N-methyl-4-[({4-[(4-methylphenyl)amino]furo[2,3-a]pyridazin-7-yl}oxy)methyl]-2-pyridinecarboxamide;
N-methyl-4-[({4-[(3-methylphenyl)amino]furo[2,3-d]pyridazin-7-yl}oxy)methyl]-2-pyridinecarboxamide;
N-methyl-4-{[(4-{[4-(trifluoromethyl)phenyl]amino}furo[2,3-a]pyridazin-7-yl)oxy]methyl}-2-pyridinecarboxamide;
N-methyl-4-{[(4-{[4-(trifluoromethoxy)phenyl]amino}furo[2,3-d]pyridazin-7-yl)oxy]methyl}-2-pyridinecarboxamide;
4-[({4-[(3-chloro-4-methoxyphenyl)amino]furo[2,3-a]pyridazin-7-yl}oxy)methyl]-N-methyl-2-pyridinecarboxamide;
4-({[4-({4-[acetyl(methyl)amino]phenyl}amino)furo[2,3-d]pyridazin-7-yl]oxy}methyl)-N-methyl-2-pyridinecarboxamide;
N-methyl-4-{[(4-{[4-(4-morpholinyl)phenyl]amino}furo[2,3-d]pyridazin-7-yl)oxy]methyl}-2-pyridinecarboxamide;
4-[({4-[(3,4-difluorophenyl)amino]furo[2,3-d]pyridazin-7-yl}oxy)methyl]-N-methyl-2-pyridinecarboxamide;
N-(1,3-benzothiazol-6-yl)-N-{4-[(4-chlorophenyl)amino]furo[2,3-d]pyridazin-7-yl}amine;
4-({[4-(2,3-dihydro-1H-inden-5-ylamino)furo[2,3-d]pyridazin-7-yl]oxy}methyl)-N-methyl-2-pyridinecarboxamide;
4-[({4-[(2-methoxyphenyl)amino]furo[2,3-d]pyridazin-7-yl}oxy)methyl]-N-methyl-2-pyridinecarboxamide;
4-[({4-[(3-methoxyphenyl)amino]furo[2,3-a]pyridazin-7-yl}oxy)methyl]-N-methyl-2-pyridinecarboxamide;
4-({[4-(1,3-benzodioxol-5-ylamino)furo[2,3-d]pyridazin-7-yl]oxy}methyl)-N-methyl-2-pyridinecarboxamide;
4-[({4-[(3,4-dichlorophenyl)amino]furo[2,3-d]pyridazin-7-yl}oxy)methyl]-N-methyl-2-pyridinecarboxamide;
4-[({4-[(3,5-dimethylphenyl)amino]furo[2,3-d]pyridazin-7-yl}oxy)methyl]-N-methyl-2-pyridinecarboxamide;
4-({[4-(1H-indazol-5-ylamino)furo[2,3-d]pyridazin-7-yl]oxy}methyl)-N-methyl-2-pyridinecarboxamide;
4-[({4-[(4-hydroxyphenyl)amino]furo[2,3-d]pyridazin-7-yl}oxy)methyl]-N-methyl-2-pyridinecarboxamide;
4-{[(4-anilinofuro[2,3-d]pyridazin-7-yl)oxy]methyl}-N-methyl-2-pyridinecarboxamide;
4-[({4-[(3-methoxy-4-methylphenyl)amino]furo[2,3-d]pyridazin-7-yl}oxy)methyl]-N-methyl-2-pyridinecarboxamide;
N-(4-chlorophenyl)-7-{[2-(4-morpholinylcarbonyl)-4-pyridinyl]methoxy}furo[2,3-d]pyridazin-4-amine;
N-methyl-4-[({4-[(2-methyl-1,3-benzothiazol-5-yl)amino]furo[2,3-d)pyridazin-7-yl}oxy)methyl]-2-pyridinecarboxamide;
4-({[4-(1,3-benzothiazol-6-ylamino)furo[2,3-d]pyridazin-7-yl]oxy}methyl)-N-methyl-2-pyridinecarboxamide trifluoroacetate;
{4-[(14-[(4-chlorophenyl)amino]furo[2,3-d]pyridazin-7-yl]oxy)methyl]-2-pyridinyl}methanol;
4-({[4-(2,3-dihydro-1-benzofuran-5-ylamino)furo[2,3-d]pyridazin-7-yl]oxy}methyl)-N-methyl-2-pyridinecarboxamide;
4-({[4-(2,3-dihydro-1-benzofuran-5-ylamino)thieno[2,3-d]pyridazin-7-yl]oxy}methyl)-N-methyl-2-pyridinecarboxamide;
4-[({4-[(4-fluorophenyl)amino]thieno[2,3-d]pyridazin-7-yl}oxy)methyl]-N-methyl-2-pyridinecarboxamide;
N-methyl-4-[({4-[(3-methylphenyl)amino]thieno[2,3-d]pyridazin-7-yl}oxy)methyl]-2-pyridinecarboxamide;
4-[({4-[(4-methoxyphenyl)amino]thieno[2,3-d]pyridazin-7-yl}oxy)methyl]-N-methyl-2-pyridinecarboxamide;
N-methyl-4-{[(4-{[4-(trifluoromethoxy)phenyl]amino}thieno[2,3-a]pyridazin-7-yl)oxy]methyl}-2-pyridinecarboxamide;
N-methyl-4-{[(4-{[4-trifluoromethyl)phenyl]amino}thieno[2,3-d]pyridazin-7-yl)oxy]methyl}-2-pyridinecarboxamide;
4-[({4-[(4-bromophenyl)amino]thieno[2,3-d]pyridazin-7-yl}oxy)methyl]-N-methyl-2-pyridinecarboxamide;
4-({[4-(2,3-dihydro-1H-inden-5-ylamino)thieno[2,3-d]pyridazin-7-yl]oxy}methyl)-N-methyl-2-pyridinecarboxamide;
4-({[4-(1,3-benzodioxol-5-ylamino)thieno[2,3-d]pyridazin-7-yl]oxy}methyl)-N-methyl-2-pyridinecarboxamide;
N-(1,3-benzothiazol-6-yl)-N-[4-(1,3-benzothiazol-6-ylamino)thieno[2,3-d]pyridazin-7-yl]amine;
N-(1,3-benzothiazol-6-yl)-N-{4-[(4-bromophenyl)amino]thieno[2,3-d]pyridazin-7-yl}amine;
N-(1,3-benzothiazol-6-yl)-N-{4-[(2,4-dimethylphenyl)amino]thieno[2,3-d]pyridazin-7-yl}amine;
N-(1,3-benzothiazol-6-yl)-N-{4-[(3-fluoro-4-methylphenyl)amino]thieno[2,3-d]pyridazin-7-yl}amine;
4-[({4-[(4-chlorophenyl)amino]furo[2,3-d]pyridazin-7-yl}oxy)methyl]-N-[2-(dimethylamino)ethyl]-2-pyridinecarboxamide;
4-[({4-[(4-chlorophenyl)amino]furo[2,3-d]pyridazin-7-yl}oxy)methyl]-N-cyclopropyl-2-pyridinecarboxamide;
4-[({4-[(4-chlorophenyl)amino]furo[2,3-d]pyridazin-7-yl}oxy)methyl]-N-(2-hydroxyethyl)-2-pyridinecarboxamide;
4-[({4-[(4-chlorophenyl)amino]furo[2,3-d]pyridazin-7-yl}oxy)methyl]-N-ethyl-2-pyridinecarboxamide;
4-[({4-[(4-chlorophenyl)amino]furo[2,3-d]pyridazin-7-yl}oxy)methyl]-N-methyl-2-pyridinecarboxamide 4-methylbenzenesulfonate;
4-[({4-[(4-chlorophenyl)amino]furo[2,3-d]pyridazin-7-yl}oxy)methyl]-N-methyl-2-pyridinecarboxamide 4-chlorobenzenesulfonate;
4-[({4-[(4-chlorophenyl)amino]furo[2,3-d]pyridazin-7-yl}oxy)methyl]-N-methyl-2-pyridinecarboxamide methanesulfonate;
4-[({4-[(4-chlorophenyl)amino]furo[2,3-d]pyridazin-7-yl}oxy)methyl]-N-methyl-2-pyridinecarboxamide ethanesulfonatesulfonate;
4-[({4-[(4-chlorophenyl)amino]furo[2,3-d]pyridazin-7-yl}oxy)methyl]-N-methyl-2-pyridinecarboxamide dihydrochloride;
4-[({4-[(4-chlorophenyl)amino]furo[2,3-d]pyridazin-7-yl}oxy)methyl]-N-methyl-2-pyridinecarboxamide hydrobromide;
4-[({4-[(4-chlorophenyl)amino]furo[2,3-d]pyridazin-7-yl}oxy)methyl]-N-methyl-2-pyridinecarboxamide sulfate;
4-[({4-[(4-chlorophenyl)amino]furo[2,3-d]pyridazin-7-yl}oxy)methyl]-N-methyl-2-pyridinecarboxamide nitrate;
4-[({4-[(4-chlorophenyl)amino]furo[2,3-d]pyridazin-7-yl}oxy)methyl]-N-methyl-2-pyridinecarboxamide 2-hydroxyethanesulfonate; and
4-[({4-[(4-chlorophenyl)amino]furo[2,3-d]pyridazin-7-yl}oxy)methyl]-N-methyl-2-pyridinecarboxamide benzenesulfonate.

12. The method of claim 10, wherein the first chemotherapeutic agent and the compound are administered simultaneously.

13. The method of claim 10, wherein the first chemotherapeutic agent and the compound are administered sequentially.

14. The method of claim 10, wherein the subject is a human.

15. The method of claim 10, wherein the subject is a non-human mammal.
